(19) United States
(12) Patent Application Publication
(10) Pub. No.: US 2026/0035742 A1
(43) Pub. Date: Feb. 5, 2026

(54) METHODS FOR THE MOLECULAR SUBTYPING OF TUMORS FROM ARCHIVAL TISSUE

(71) Applicants: BioVentures, LLC, Little Rock, AR (US); University of Southern California, Los Angeles, CA (US)

(72) Inventors: Donald Johann, Little Rock, AR (US); James Hicks, Los Angeles, CA (US)

(21) Appl. No.: 18/997,172

(22) PCT Filed: Jun. 21, 2023
(86) PCT No.: PCT/US2023/068839

§ 371 Date: Jan. 20, 2025

Related U.S. Application Data
(60) Provisional application No. 63/354,128, filed on Jun. 21, 2022.

(51) Int. Cl. C12Q 1/686; C12Q 1/37; C12Q 1/6806; C12Q 1/6886

(52) U.S. Cl. CPC C12Q 1/686; C12Q 1/37; C12Q 1/6806; C12Q 1/6886; C12Q 2600/106; C12Q 2600/112; C12Q 2600/158; C12Y 301/21001

(57) ABSTRACT

The present disclosure encompasses methods for molecularly subtyping formalin-fixed paraffin-embedded tumor samples. The disclosure works particularly well for old and degraded (archival) samples for which standard methods are unfeasible. Further, the methods disclosed allow for the correlation of patient outcome data with the molecular subtype of the tumor and provides a wealth of information which will guide treatment decisions and/or selection of therapeutic agents.

### CROSS REFERENCE TO RELATED APPLICATIONS

The present application claims the benefit of U.S. Provisional Patent Application No. 63/354,128, entitled, “METHODS FOR THE MOLECULAR SUBTYPING OF TUMORS FROM ARCHIVAL TISSUE” filed Jun. 21, 2022. The content of the aforementioned application is hereby incorporated by reference in its entirety.

### FIELD OF THE TECHNOLOGY

The present disclosure encompasses methods for the identification of the molecular subtype of a cancer or tumor. Further, the methods are useful for archival samples that are aged and typically of a degraded condition.

### BACKGROUND

Molecular studies are now routinely performed in pathology laboratories for the diagnosis of various medical conditions such as cancer, infectious diseases and genetic disorders. One of the most commonly performed molecular tests is the polymerase chain reaction (PCR) which enables the amplification of specific sequences of nucleic acids from an extremely small amount of genetic starting material. In most laboratories, PCR is usually performed on a variety of fresh specimens including blood, body fluids and tissues. There are instances when fresh material is not available and there is an unmet biomedical science need for the performance of PCR on archival material. This principle may be extremely important when a diagnosis is unsuspected and when fresh tissue is no longer available, as well as for the testing of remotely obtained samples. However, the suitability of archival material may be questioned as the probes used in assays such as i) Oncotype Dx Recurrence Score, ii) Prosigna Predicition Analysis of Microarray 50 (PAM50) Risk of Recurrence, iii) EndoPredict, iv) MamaPrint and v) Breast Cancer Index do not work well on archival material especially for samples that are old and of a degraded condition.

In contrast to fresh samples, archival material such as cytological specimens, paraffin-embedded, or frozen tissues, presents the opportunity for a careful morphologic review and interpretation prior to molecular analysis. This allows the trained cytologist to preselect cases and slide preparations for subsequent molecular analysis, resulting in optimal utilization and cost control in the molecular laboratory. As molecular techniques are improved and refined, diagnostic possibilities will become realities.

Both clinicians and pathologists have operated under the assumption that fresh specimens have the best diagnostic yield for molecular studies. However, fresh material is not always available to perform molecular testing, and careful comparative studies are few. There is, therefore, a need in the art for a method to assess archival tissues used as diagnostic samples. This is of increased importance when clinical outcome data are available and linked to these samples and, will allow for the running and analyses of retrospective clinical trials using molecular profiling data from these “now available to be analyzed” samples.

### SUMMARY

In some aspects, the disclosure encompasses a method of molecular subtyping a cancer sample obtained from a subject, the method comprising (a) enhanced solubilization of the old and degraded FFPE sample material through the non-discretionary use of mineral oil, (b) digesting the sample with a proteinase, (c) incubating the digested sample from step a) with a DNase, (d) incubating the mixture from step b) with a guanidine salt based buffer, (e) concentrating and isolating the RNA, (f) pre-amplifying, and (g) performing digital droplet PCR.

In some aspects, the sample is a formalin-fixed and paraffin-embedded sample. In some aspects, the sample is at least 5 years old or older.

In some aspects, the sample is digested with proteinase K. In some aspects, the sample is digested at about 65° C. to about 70° C. for about 75 minutes.

In some aspects, the step a) further comprises separating the sample into an aqueous phase by centrifugation and separating the aqueous phase from a residual lysate, where the aqueous phase is used for step b).

In some aspects of the method, the RNA is concentrated and isolated using a spin column. In some aspects, the residual lysate is used for DNA extraction.

In some aspects of the method, proteinase is incubated with the residual lysate at about 65° C. to about 70° C. for about 13 hours to about 18 hours thereby producing a digested tissue lysate. In some aspects, the digested tissue lysate is incubated with an RNase.

Another aspect of the method further comprises concentrating and isolating DNA using a spin column. In some aspects, the method further comprises the step of pre-amplifying the isolated DNA and performing ddPCR.

In some aspects of the method, a target and optionally a reference nucleic acid are quantitated. In some aspects, the target nucleic acid or fragment thereof encodes a Estrogen receptor 1 (ESR1), Progesterone receptor (PGR), B-cell lymphoma 2 (BCL2), Signal Peptide, CUB Domain And EGF Like Domain Containing 2 (SCUBE2), human epidermal growth factor receptor 2 (HER2), Growth factor receptor-bound protein 7 (GRB7), Marker Of Proliferation Ki-67 (MK167), Aurora kinase A (AURKA), Baculoviral IAP Repeat Containing 5 (BIRC5), Cyclin B1 (CCNB1), MYB Proto-Oncogene Like 2 (MYBL2), Thymidine kinase 1 (TK1), or any combination thereof.

In some aspects, the cancer sample is breast cancer sample. In some aspects, the subtype comprises a Luminal A subtype (Lum A), Luminal B subtype (Lum B), HER2 subtype (HER2) or Triple Negative subtype (TN).

In some aspects, the sample is determined to be Lum A if the level of one or more of target nucleic acid or fragment thereof encoding ESR1, PGR, BCL2, SCUBE2, or any combination thereof, is elevated in the sample.

In some aspects, the sample is determined to be Lum B if the level of nucleic acid or fragment thereof encoding ESR1, PGR, BCL2, SCUBE2, or any combination thereof, is elevated, and if the level of nucleic acid or fragment thereof encoding MK167, AURKA, BIRC5, CCNB1, MYBL2, TK1, or any combination thereof is elevated in the sample.

In some aspects, the sample is determined to be HER2 if the level of nucleic acid or fragment thereof encoding HER2, GRB7, or any combination thereof is elevated in the sample.

In some aspects, the sample is determined to be TN if the level of nucleic acid or fragment thereof encoding MK167, AURKA, BIRC5, CCNB1, MYBL2, TK1, or any combination thereof is elevated in the sample.

In further aspects, the amount of target nucleic acid is compared to the subject outcome or a therapeutic response.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A depicts the S8897 specimen archive used in the examples. The S8897 specimen archive contains material from the largest number of patients not treated following surgery for small breast tumors. It is estimated that many patient specimens involve ˜4-6 glass slides of unstained formalin fixed paraffin embedded (FFPE) material that are over ˜20-30 years old. The low-risk group criteria were: Patients with T<1 cm that did not have HR status or S phase flow cytometry evaluated (aka Initial Low Risk Group). Specimens involve glass slides of unstained FFPE material.

FIG. 1B depicts flow diagram of the archival specimens and their molecular profiling history.

FIG. 2A-2C show representative Fragment Analyzer (FA) Traces of Specimens (Extracted Nucleic Acids). FIG. 2A shows a representative RNA Fragment Analyzer Traces of a breast tumor specimen. FIG. 2B shows a representative RNA Fragment Analyzer Traces of a normal LN. FIG. 2C shows a representative genomic DNA Fragment Analyzer Traces of a breast tumor specimen. Similar RNA Fragment Analyzer Traces was performed for all the tumor specimen T1-T33 (data not shown).

FIG. 3A-3D show breast cancer tumor subtype determination for samples T15, T19, T23, and T21. x-axis represents the molecular targets and the y-axis represents the z-score transform of ddPCR counts. FIG. 3A shows specimen T15 (Lum A). FIG. 3B shows specimen T19 (Lum B). FIG. 3C shows specimen T23 (Her 2). FIG. 3D shows specimen T21 (TN).

FIG. 4A-4U show breast cancer tumor subtype determination using ddPCR for samples. x-axis represents the molecular targets and the y-axis represents the z-score transform of ddPCR counts. FIG. 4A shows specimen T1. FIG. 4B shows specimen T2. FIG. 4C shows specimen T3 (Her 2). FIG. 4D shows specimen T4. FIG. 4E shows specimen T5. FIG. 4F shows specimen T6. FIG. 4G shows specimen T7. FIG. 4H shows specimen T8. FIG. 4I shows specimen T9. FIG. 4J shows specimen T10. FIG. 4K shows specimen T11. FIG. 4L shows specimen T12. FIG. 4M shows specimen T13. FIG. 4N shows specimen T14. FIG. 4O shows specimen T16. FIG. 4P shows specimen T17. FIG. 4Q shows specimen T18. FIG. 4R shows specimen T20.

FIG. 4S shows specimen T22. FIG. 4T shows specimen T24. FIG. 4U shows specimen T25.

FIG. 5A-5F shows show distance geometry. FIG. 5A displays a specimen-based HCA of the 25 breast cancer specimens. FIG. 5B shows the same 25 specimens following PCA. FIG. 5C illustrates the HCA of the same 25 breast cancer specimens on the x-axis versus the 12 molecular markers used by the ddPCR assay.

FIG. 5D-5F show the results of 25 breast tumor specimens (T1-T25) along with the addition of 13 normal human lymph node specimens (LN1-LN13) from the S8897 cohort following processing and analysis by the ddPCR assay.

FIG. 6A-6F show statistical simulation of specimens T1-T25 and LN1-LN13. Molecular profiling was based on breast tumor RNA. FIG. 6A displays a specimen-based HCA of the T1-T25 and synthetic breast cancer specimens. FIG. 6B shows the results of PCA involving the T1-T25 along 500 synthetic specimens for each PAM50 subtype. FIG. 6C illustrates the HCA of the T1-T25 specimens with 500 synthetic specimens for each PAM50 subtype. FIG. 6D displays a specimen-based HCA of the T1-T25 and LN1-LN13 specimens, and synthetic specimens. FIG. 6E shows the results of PCA involving the T1-T25 and LN1-LN13 along 500 synthetic specimens for each PAM50 subtype and lymph node. FIG. 6F illustrates the HCA of the T1-T25 and LN1-LN13 specimens with 500 synthetic specimens for each PAM50 subtype and lymph node on the x-axis versus the 12 molecular markers used by the ddPCR assay.

FIG. 7A-7B show clinical and genomic features. FIG. 7A display clinical and genomic features. FIG. 7B show IntClust Related Chromosomal Bands and Associated Copy Numbers.

FIG. 8A-8C show DNA specimen analysis of sample T6. FIG. 8A shows UAMS 20 year-old tumor, used ichor pooled normal for CNV (no matched normal available) Initial Specimen ID, S98-9; Final Specimen ID, T6; HistoPath, ILC; Grade 1; % Tumor=60%; Molec subtype (ddPCR), Lum A. FIG. 8B shows IntClust related chromosomal bands and associated copy numbers (Basal via InClust 10). FIG. 8C shows top 27 targets from UMI-based gene panel with mutation impact color coding.

FIG. 9A-9C show DNA specimen analysis of sample T1. FIG. 9A shows UAMS 20 year-old tumor, used ichor pooled normal for CNV (no matched normal available) Initial Specimen ID, S98-3; Final Specimen ID, T1; HistoPath, IDC; Grade 3; % Tumor=30%; Molec subtype (ddPCR), Lum B. FIG. 9B shows IntClust related chromosomal bands and associated copy numbers (LumA via InClust 8). FIG. 9C shows top 27 targets from UMI-based gene panel with mutation impact color coding.

FIG. 10A-10C show DNA specimen analysis of sample T2. FIG. 10A shows UAMS 20 year-old tumor, used ichor pooled normal for CNV (no matched normal available) Initial Specimen ID, S98-4; Final Specimen ID, T2; HistoPath, IDC; Grade 3; % Tumor=40%; Molec subtype (ddPCR), TN. FIG. 10B shows IntClust related chromosomal bands and associated copy numbers (Basal via InClust 10). FIG. 10C shows top 27 targets from UMI-based gene panel with mutation impact color coding.

FIG. 11A-11C show DNA specimen analysis of sample T3. FIG. 11A shows UAMS 20 year-old tumor, used ichor pooled normal for CNV (no matched normal available) Initial Specimen ID, S98-5; Final Specimen ID, T3; HistoPath, IDC; Grade 3; % Tumor=40%; Molec subtype (ddPCR), TN. FIG. 11B shows IntClust related chromosomal bands and associated copy numbers (Basal via InClust 10). FIG. 11C shows top 27 targets from UMI-based gene panel with mutation impact color coding.

FIG. 12A-12C show DNA specimen analysis of sample T4. FIG. 12A shows UAMS 20 year-old tumor, used ichor pooled normal for CNV (no matched normal available) Initial Specimen ID, S98-6; Final Specimen ID, T4; HistoPath, IDC; Grade 3; % Tumor=40%; Molec subtype (ddPCR), Lum B. FIG. 12B shows IntClust related chromosomal bands and associated copy numbers (Basal via InClust 10 vs Lum B via InClust1, 2). FIG. 12C shows top 27 targets from UMI-based gene panel with mutation impact color coding.

FIG. 13A-13C show DNA specimen analysis of sample T5. FIG. 13A shows UAMS 20 year-old tumor, used ichor pooled normal for CNV (no matched normal available) Initial Specimen ID, S98-8; Final Specimen ID, T5; HistoPath, IDC; Grade 1; % Tumor=70%; Molec subtype (ddPCR), Lum A. FIG. 13B shows IntClust related chromosomal bands and associated copy numbers (Lum A via InClust 8). FIG. 13C shows top 27 targets from UMI-based gene panel with mutation impact color coding.

FIG. 14A-14C show DNA specimen analysis of sample T7. FIG. 14A shows UAMS 20 year-old tumor, used ichor pooled normal for CNV (no matched normal available) Initial Specimen ID, S98-11; Final Specimen ID, T7; HistoPath, LN Met; Grade 2; % Tumor=50%; Molec subtype (ddPCR), Lum A; B. FIG. 14B shows IntClust related chromosomal bands and associated copy numbers (Lum A via InClust 8; 9). FIG. 14C shows top 27 targets from UMI-based gene panel with mutation impact color coding.

FIG. 15A-15C show DNA specimen analysis of sample T8. FIG. 15A shows UAMS 20 year-old tumor, used ichor pooled normal for CNV (no matched normal available) Initial Specimen ID, S98-12; Final Specimen ID, T8; HistoPath, ILC; Grade 2; % Tumor=50%; Molec subtype (ddPCR), Lum A. FIG. 15B shows IntClust related chromosomal bands and associated copy numbers (Lum A; B via InClust 7; 1, 2, 6). FIG. 15C shows top 27 targets from UMI-based gene panel with mutation impact color coding.

FIG. 16A-16C show DNA specimen analysis of sample T9. FIG. 16A shows UAMS 20 year-old tumor, used ichor pooled normal for CNV (no matched normal available) Initial Specimen ID, S99-7; Final Specimen ID, T9; HistoPath, DCIS; Grade 2; % Tumor=30%; Molec subtype (ddPCR), Lum A. FIG. 16B shows IntClust related chromosomal bands and associated copy numbers (Lum A; Her2 via InClust 7; 8, 5). FIG. 16C shows top 27 targets from UMI-based gene panel with mutation impact color coding.

FIG. 17A-17C show DNA specimen analysis of sample T10. FIG. 17A shows UAMS 20 year-old tumor, used ichor pooled normal for CNV (no matched normal available) Initial Specimen ID, S99-16; Final Specimen ID, T10; HistoPath, IDS; Grade 3; % Tumor=60%; Molec subtype (ddPCR), TN. FIG. 17B shows IntClust related chromosomal bands and associated copy numbers (Basal via InClust 10). FIG. 17C shows top 27 targets from UMI-based gene panel with mutation impact color coding.

FIG. 18A-18C show DNA specimen analysis of sample T11. FIG. 18A shows UAMS 20 year-old tumor, used ichor pooled normal for CNV (no matched normal available) Initial Specimen ID, S99-17; Final Specimen ID, T11; HistoPath, IDC; Grade 3; % Tumor=80%; Molec subtype (ddPCR), TN. FIG. 18B shows IntClust related chromosomal bands and associated copy numbers (Basal via InClust 10). FIG. 18C shows top 27 targets from UMI-based gene panel with mutation impact color coding.

FIG. 19A-19C show DNA specimen analysis of sample T12. FIG. 19A shows UAMS 20 year-old tumor, used ichor pooled normal for CNV (no matched normal available) Initial Specimen ID, S99-18; Final Specimen ID, T12; HistoPath, IDC; Grade 3; % Tumor=50%; Molec subtype (ddPCR), TN. FIG. 19B shows IntClust related chromosomal bands and associated copy numbers (Basal via InClust 10). FIG. 19C shows top 27 targets from UMI-based gene panel with mutation impact color coding.

FIG. 20A-20C show DNA specimen analysis of sample T13. FIG. 20A shows UAMS 20 year-old tumor, used ichor pooled normal for CNV (no matched normal available) Initial Specimen ID, S99-19; Final Specimen ID, T13; HistoPath, IDC; Grade 3; % Tumor=70%; Molec subtype (ddPCR), TN. FIG. 20B shows IntClust related chromosomal bands and associated copy numbers (Basal via InClust 10). FIG. 20C shows top 27 targets from UMI-based gene panel with mutation impact color coding.

FIG. 21A-21C show DNA specimen analysis of sample T14. FIG. 21A shows S8897 Tumor vs Matched LN. Initial Specimen ID, Tu2; Final Specimen ID, T14; HistoPath, IDC; Grade 2; % Tumor=50%; Molec subtype (ddPCR), Lum A. FIG. 21B shows IntClust related chromosomal bands and associated copy numbers (LumA via InClust 7). FIG. 21C shows top 27 targets from UMI-based gene panel with mutation impact color coding.

FIG. 22A-22C show DNA specimen analysis of sample T15. FIG. 22A shows S8897 Tumor vs Matched LN. Initial Specimen ID, Tu3; Final Specimen ID, T15; HistoPath, IDC; Grade 2; % Tumor=95%; Molec subtype (ddPCR), Lum A. FIG. 22B shows IntClust related chromosomal bands and associated copy numbers (LumA via InClust 7). FIG. 22C shows top 27 targets from UMI-based gene panel with mutation impact color coding.

FIG. 23A-23C show DNA specimen analysis of sample T16. FIG. 23A shows S8897 Tumor vs Matched LN. Initial Specimen ID, Tu11; Final Specimen ID, T16; HistoPath, IDC; Grade 2; % Tumor=20%; Molec subtype (ddPCR), Lum A. FIG. 23B shows IntClust related chromosomal bands and associated copy numbers (Lum A, B via InClust 8; 2). FIG. 23C shows top 27 targets from UMI-based gene panel with mutation impact color coding.

FIG. 24A-24B show DNA specimen analysis of sample T17. FIG. 24A shows S8897 Tumor vs Matched LN. Initial Specimen ID, Tu12; Final Specimen ID, T17; HistoPath, ADH, ALH; Grade NA; % Tumor=0%; Molec subtype (ddPCR), NA. FIG. 24B shows IntClust related chromosomal bands and associated copy numbers.

FIG. 25A-25C show DNA specimen analysis of sample T18. FIG. 25A shows S8897 Tumor vs Matched LN. Initial Specimen ID, Tu13; Final Specimen ID, T18; HistoPath, DCIS & IDC; Grade 1; % Tumor=20%; Molec subtype (ddPCR), Lum A.

FIG. 25B shows IntClust related chromosomal bands and associated copy numbers (Lum A, via InClust 7; 8). FIG. 25C shows top 27 targets from UMI-based gene panel with mutation impact color coding.

FIG. 26A-26C show DNA specimen analysis of sample T19. FIG. 26A shows S8897 Tumor vs Matched LN. Initial Specimen ID, Tu15; Final Specimen ID, T19; HistoPath, IDC; Grade 2; % Tumor=20%; Molec subtype (ddPCR), Lum B. FIG. 26B shows IntClust related chromosomal bands and associated copy numbers (Lum B, via InClust 8). FIG. 26C shows top 27 targets from UMI-based gene panel with mutation impact color coding.

FIG. 27A-27C show DNA specimen analysis of sample T20. FIG. 27A shows S8897 Tumor vs Matched LN. Initial Specimen ID, Tu16; Final Specimen ID, T20; HistoPath, DCIS & IDC (4:1); Grade 1; % Tumor=25%; Molec subtype (ddPCR), Lum A. FIG. 27B shows IntClust related chromosomal bands and associated copy numbers (Lum A, via InClust 8). FIG. 27C shows top 27 targets from UMI-based gene panel with mutation impact color coding.

FIG. 28A-28C show DNA specimen analysis of sample T21. FIG. 28A shows S8897 Tumor vs Matched LN. Initial Specimen ID, Tu18; Final Specimen ID, T21; HistoPath, IDC; Grade 2; % Tumor less than 5%; Molec subtype (ddPCR), TN. FIG. 28B shows IntClust related chromosomal bands and associated copy numbers (Basal vs Lum B, via InClust 10, 9). FIG. 28C shows top 27 targets from UMI-based gene panel with mutation impact color coding.

FIG. 29A-29C show DNA specimen analysis of sample T22. FIG. 29A shows S8897 Tumor vs Matched LN. Initial Specimen ID, Tu19; Final Specimen ID, T22; HistoPath, IDC; Grade 1; % Tumor=100%; Molec subtype (ddPCR), Lum A. FIG. 29B shows IntClust related chromosomal bands and associated copy numbers (Lum A, via InClust 7). FIG. 29C shows top 27 targets from UMI-based gene panel with mutation impact color coding.

FIG. 30A-30B show DNA specimen analysis of sample T23. FIG. 30A shows S8897 Tumor vs Matched LN. Initial Specimen ID, Tu20; Final Specimen ID, T23; HistoPath, DCIS & IDC (4:1); Grade 2; % Tumor=50%; Molec subtype (ddPCR), HER2. FIG. 30B shows IntClust related chromosomal bands and associated copy numbers (HER2 via InClust 5).

FIG. 31A-31C show DNA specimen analysis of sample T26. FIG. 31A shows S8897 Tumor vs Matched LN. Initial Specimen ID, Tu1; Final Specimen ID, T26; HistoPath, IDC; Grade 1; % Tumor=90%. FIG. 31B shows IntClust related chromosomal bands and associated copy numbers (Lum A via InClust 8). FIG. 31C shows top 27 targets from UMI-based gene panel with mutation impact color coding.

FIG. 32A-32C show DNA specimen analysis of sample T27. FIG. 32A shows S8897 Tumor vs Matched LN. Initial Specimen ID, Tu4; Final Specimen ID, T27; HistoPath, IDC; Grade 2; % Tumor=100%. FIG. 32B shows IntClust related chromosomal bands and associated copy numbers (Lum A via InClust 7 and 8). FIG. 32C shows top 27 targets from UMI-based gene panel with mutation impact color coding.

FIG. 33A-33C show DNA specimen analysis of sample T28. FIG. 33A shows S8897 Tumor vs Matched LN. Initial Specimen ID, Tu5; Final Specimen ID, T28; HistoPath, IDC; Grade 1; % Tumor=60%. FIG. 33B shows IntClust related chromosomal bands and associated copy numbers (Lum A via InClust 8). FIG. 33C shows top 27 targets from UMI-based gene panel with mutation impact color coding.

FIG. 34A-34C show DNA specimen analysis of sample T29. FIG. 34A shows S8897 Tumor vs Matched LN. Initial Specimen ID, Tu6; Final Specimen ID, T29; HistoPath, IDC & focal DCIS; Grade 2; % Tumor=95%. FIG. 34B shows IntClust related chromosomal bands and associated copy numbers (Lum A via InClust 7). FIG. 34C shows top 27 targets from UMI-based gene panel with mutation impact color coding.

FIG. 35A-35C show DNA specimen analysis of sample T30. FIG. 35A shows S8897 Tumor vs Matched LN. Initial Specimen ID, Tu7; Final Specimen ID, T30; HistoPath, IDC & DCIS; Grade 3; % Tumor=40%. FIG. 35B shows IntClust related chromosomal bands and associated copy numbers (Lum A, B via InClust 7; 8, 9). FIG. 35C shows top 27 targets from UMI-based gene panel with mutation impact color coding.

FIG. 36A-36C show DNA specimen analysis of sample T31. FIG. 36A shows S8897 Tumor vs Matched LN. Initial Specimen ID, Tu8; Final Specimen ID, T31; HistoPath, IDC; Grade 3; % Tumor=30%. FIG. 36B shows IntClust related chromosomal bands and associated copy numbers (Lum B via InClust 2). FIG. 36C shows top 27 targets from UMI-based gene panel with mutation impact color coding.

FIG. 37A-37C show DNA specimen analysis of sample T32. FIG. 37A shows S8897 Tumor vs Matched LN. Initial Specimen ID, Tu10; Final Specimen ID, T32; HistoPath, IDC & DCIS (9:1); Grade 2; % Tumor=100%. FIG. 37B shows IntClust related chromosomal bands and associated copy numbers (Lum B via InClust 1). FIG. 37C shows top 27 targets from UMI-based gene panel with mutation impact color coding.

FIG. 38A-38C show DNA specimen analysis of sample T33. FIG. 38A shows S8897 Tumor vs Matched LN. Initial Specimen ID, Tu14; Final Specimen ID, T33; HistoPath, DCIS; Grade 2; % Tumor=15%. FIG. 38B shows IntClust related chromosomal bands and associated copy numbers (Lum A/B). FIG. 38C shows top 27 targets from UMI-based gene panel with mutation impact color coding.

FIG. 39 shows the copy number mapping criteria to PAM50 subtypes.

FIG. 40 shows specimen T1, S98-3, IHC for ER→ER negative.

FIG. 41 shows specimen T2, S98-4, S98-3292 A3 H&E stain.

FIG. 42 shows specimen T2, S98-4, ER→ER negative.

FIG. 43 shows specimen T3, S98-5, S98-3292 A4 H&E stain.

FIG. 44 shows specimen T3, S98-5, IHC for ER→ER negative.

FIG. 45 shows specimen T4, S98-6, S98-3292 A5 H&E stain.

FIG. 46 shows specimen T4, S98-6, IHC for ER→ER negative.

FIG. 47 shows specimen T5, S98-8, S98-8029 A3 H&E stain.

FIG. 48 shows specimen T5, S98-8, IHC for ER→ER positive.

FIG. 49 shows specimen T6, S98-9, S98-8029 A4 H&E stain.

FIG. 50 shows specimen T6, S98-9, IHC for ER→ER positive.

FIG. 51 shows specimen T7, S98-11, S98-8045 A1 H&E stain.

FIG. 52 shows specimen T6, S98-11, IHC for ER→ER positive.

FIG. 53 shows specimen T8, S98-12, S98-11141 C1 H&E stain.

FIG. 54 shows specimen T8, S98-12, IHC for ER. IHC was inconclusive due to extensive necrosis and crush artifact.

FIG. 55 shows specimen T1, S98-3, S98-3292 A1 H&E stain.

FIG. 56 shows specimen T9, S99-7, S99-4808 A3 H&E stain.

FIG. 57 shows specimen T9, S99-7, IHC for ER→ER negative.

FIG. 58 shows specimen T9, S99-7, IHC for HER2 positive→HER2 positive.

FIG. 59 shows specimen T10, S99-16, S99-14088 C10 H&E stain.

FIG. 60 shows specimen T10, S99-16, IHC for ER →ER negative.

FIG. 61 shows specimen T11, S99-17, S99-14088 C11 H&E stain.

FIG. 62 shows specimen T11, S99-17, IHC for ER →ER negative.

FIG. 63 shows specimen T12, S99-18, S99-14088 C12 H&E stain.

FIG. 64 shows specimen T12, S99-18, IHC for ER →ER negative.

FIG. 65 shows specimen T13, S99-19, S99-14088 C13 H&E stain.

FIG. 66 shows specimen T13, S99-19, IHC for ER →ER negative.

### DETAILED DESCRIPTION

The disclosure provided herein is partly based on the discovery and development of methods that can be used for assessing specific signatures (e.g., risk of recurrence) based on tumor subtypes and markers in archived specimens (20-30 years-old). The present disclosure provides an in vitro RNA-based, multi-parameter molecular diagnostic method for the identification of the molecular subtype of a tumor or cancer from a subject. As the cancer clinical diagnosis is further defined, the prognosis may be better determined, and the predictability of therapeutic response is better established by identifying a subject's cancer or tumor subtype and correlating that information with treatment outcomes. Tissue from active subjects or archival samples may be used for subtyping. When outcome data are available archival samples, they offer a rich source of discovery, but to date current approaches do not work well on archival samples that are old and degraded. There are tissue banks containing archival samples with outcome data that could be used as a retrospective source of discovery if a suitable molecular diagnostic method existed for effective subtyping. Further, such samples can be used to identify the circumstances when toxic adjuvant therapy can be avoided.

### I. Definitions

So that the present disclosure may be more readily understood, certain terms are first defined. Unless defined otherwise, all technical and scientific terms used herein have the same meaning as commonly understood by one of ordinary skill in the art to which aspects of the disclosure pertain. Many methods and materials similar, modified, or equivalent to those described herein can be used in the practice of the various aspects of the present disclosure without undue experimentation, the preferred materials and methods are described herein. In describing and claiming the aspects of the present disclosure, the following terminology will be used in accordance with the definitions set out below.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, “individual”, “subject”, “host”, and “patient” can be used interchangeably and may refer to any human or non-human mammalian subject for whom diagnosis, treatment, prophylaxis or therapy is desired, for example, humans, pets, livestock, horses or other animals. In some aspects, the subject is a human. In other aspects, the subject is a human having cancer (e.g., breast cancer), including those who have undergone or are candidates for resection (surgery) to remove cancerous tissue.

In one aspect, the subject may be a rodent, e.g. a mouse, a rat, a guinea pig, etc. In another aspect, the subject may be a livestock animal. Non-limiting examples of suitable livestock animals may include pigs, cows, horses, goats, sheep, llamas and alpacas. In still another aspect, the subject may be a companion animal. Non-limiting examples of companion animals may include pets such as dogs, cats, and rabbits. In yet another aspect, the subject may be a zoological animal. As used herein, a “zoological animal” refers to an animal that may be found in a zoo. Such animals may include non-human primates, large cats, wolves, and bears. In a preferred aspect, the subject is a human.

As used herein, the term “surgery” applies to surgical methods undertaken for removal of cancerous tissue, including mastectomy, lumpectomy, lymph node removal, sentinel lymph node dissection, prophylactic mastectomy, prophylactic ovary removal, cryo-therapy, and tumor biopsy. The tumor samples used for the methods of the present invention may have been obtained from any of these methods.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition, or disorder or those in which the disease, condition or disorder is to be prevented.

As used herein “cancer,” “tumor,” or “malignancy” may refer to one or more neoplasm or cancer. The neoplasm may be malignant or benign, the cancer may be primary or metastatic; the neoplasm or cancer may be early stage or late stage. Non-limiting examples of neoplasms or cancers may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, and Wilms tumor (childhood).

As used herein, “sample”, “biological sample” or “specimen” may be of any biological tissue, fluid, or cell from the subject. The term “tumor” as used herein, refers to all neoplastic cell growth and proliferation, whether malignant or benign, and all pre-cancerous and cancerous cells and tissues. The term “tumor sample” as used herein refers to a sample comprising tumor material obtained from a cancer subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. A sample can be a tumor tissue, tissue surrounding a tumor, or non-tumor tissue. The tumor sample can be a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells. The term also encompasses cells that are the progeny of the subject's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a subject's tumor material. Methods of procuring a biological sample from a subject are well known in the art.

Sample from the subject can be procured one or more times, before, during and/or after diagnosis. In some aspects, samples can be procured from the subject before, during, and/or after treatment of cancer. In some aspects, control sample can be procured from a healthy subject. In some aspects, the control sample can comprise non-cancer cells. In some aspects, the non-cancer cells can be from the same tissue type as the cancer cells. For example, if the cancer cells are from breast cancer, then the non-cancer cells can be from healthy breast tissue. In some aspects, the control can comprise an average levels of the molecular profile in a sample from a subject before onset of cancer. In some aspects, control sample can be a sample from the subject prior to diagnosis or treatment. In certain aspects, the molecular profile can be measured in a person or persons other than the subject with cancer. In some aspects, the control a person or persons with similar characteristics to the subject with cancer. In some aspects, the control can be an average of the combination of disclosed molecular profile levels from different healthy sources (e.g., more than one healthy control subject). In some aspects, the control sample can be pooled sample.

As used herein “nucleic acid” can refer to a polymer comprising ribonucleosides and/or deoxyribonucleosides that are covalently bonded, typically by phosphodiester linkages between subunits, but in some cases by phosphorothioates, methylphosphonates, and the like. Examples of nucleic acids include genomic DNA; circular DNA; low molecular weight DNA, plasmid DNA; circulating DNA, circulating tumor DNA (ctDNA); hnRNA; mRNA; noncoding RNA including rRNA, tRNA, micro RNA, small interfering RNA, small nucleolar RNA, small nuclear RNA and small temporal RNA; fragmented or degraded nucleic acids; PNAs; nucleic acid obtained from subcellular organelles such as mitochondria or chloroplasts; and nucleic acid obtained from microorganisms, parasites, or DNA or RNA viruses that may be present in a biological sample.

As used herein, the term “level” “amount”, or “abundance” as used herein refers to qualitative or quantitative determination of the number of copies of a coding or non-coding RNA transcript or a polypeptide/protein, or an analyte. An RNA transcript or a polypeptide/protein exhibits an “increased level” when the level of the RNA transcript or polypeptide/protein is higher in a first sample, such as in a clinically relevant sub-population of patients (e.g., patients who have experienced cancer recurrence), than in a second sample, such as in a related subpopulation (e.g., patients who did not experience cancer recurrence). In the context of an analysis of a level of an RNA transcript or a polypeptide/protein in a tumor sample obtained from an individual patient, an RNA transcript or polypeptide/protein exhibits “increased level” when the level of the RNA transcript or polypeptide/protein in the subject trends toward, or more closely approximates, the level characteristic of a clinically relevant sub-population of patients. The amount may be a concentration, number, ratio, proportion, or a percentage of the analyte compared to the control sample or determined using a standard curve. The amount may be an absolute amount or a relative amount.

Thus, for example, when the RNA transcript analyzed is an RNA transcript that shows an increased level in subjects that experienced long-term survival without cancer recurrence as compared to subjects that did not experience long-term survival without cancer recurrence, then an “increased” level of a given RNA transcript can be described as being positively correlated with a likelihood of long-term survival without cancer recurrence. If the level of the RNA transcript in an individual patient being assessed trends toward a level characteristic of a subject who experienced long-term survival without cancer recurrence, the level of the RNA transcript supports a determination that the individual patient is more likely to experience long-term survival without cancer recurrence. If the level of the RNA transcript in the individual patient trends toward a level characteristic of a subject who experienced cancer recurrence, then the level of the RNA transcript supports a determination that the individual patient is more likely to experience cancer recurrence.

As used herein “degraded sample” sample that may be compromised or more degraded than a normal sample used for expression analysis, and has some degree of degradation. Biopsy samples from tumors are routinely stored after surgical procedures by FFPE, which may compromise DNA and RNA integrity. In some aspects, degraded sample comprise a FFPE sample.

As used herein “archived” or “archival” sample is a paraffin embedded and/or fixed tissue biopsy or a paraffin embedded and/or fixed tissue section or parts thereof, for example microdissected samples.

As used herein “biopsy” refers to any kind of needle biopsy or any kind of tissue sample collected during a surgery.

As used herein “tissue section” refers to any part of a biopsy for example derived by microtome sectioning of the biopsy.

The term “likelihood score” is an arithmetically or mathematically calculated numerical value for aiding in simplifying or disclosing or informing the analysis of more complex quantitative information, such as the correlation of certain levels of the disclosed RNA transcripts, their expression products, or gene networks to a likelihood of a certain clinical outcome in a breast cancer patient, such as likelihood of long-term survival without breast cancer recurrence. A likelihood score may be determined by the application of a specific algorithm. The algorithm used to calculate the likelihood score may group the RNA transcripts, or their expression products, into gene networks. A likelihood score may be determined for a gene network by determining the level of one or more RNA transcripts, or an expression product thereof, and weighting their contributions to a certain clinical outcome such as recurrence. A likelihood score may also be determined for a patient. In an aspect, a likelihood score is a recurrence score, wherein an increase in the recurrence score negatively correlates with an increased likelihood of long-term survival without breast cancer recurrence. In other words, an increase in the recurrence score correlates with bad prognosis. Examples of methods for determining the likelihood score or recurrence score are disclosed in U.S. Pat. No. 7,526,387.

The term “long-term” survival as used herein refers to survival for at least 3 years. In other aspect, it may refer to survival for at least 5 years, or for at least 10 years following surgery or other treatment.

As used herein, the term “normalized” with regard to a coding or non-coding RNA transcript, or an expression product of the coding RNA transcript, refers to the level of the RNA transcript, or its expression product, relative to the mean levels of transcript/product of a set of reference RNA transcripts, or their expression products. The reference RNA transcripts, or their expression products, are based on their minimal variation across patients, tissues, or treatments. Alternatively, the coding or non-coding RNA transcript, or its expression product, may be normalized to the totality of tested RNA transcripts, or a subset of such tested RNA transcripts.

As used herein, the term “pathology” of cancer includes all phenomena that comprise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes.

A “subject response” may be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the cancer; (8) increase in the length of survival following treatment; and/or (9) decreased mortality at a given point of time following treatment.

The term “prognosis” as used herein, refers to the prediction of the likelihood of cancer-attributable death or progression, including recurrence, metastatic spread, and drug resistance, of neoplastic disease, such as breast cancer. The term “prediction” is used herein to refer to the likelihood that a patient will respond either favorably or unfavorably to a drug or set of drugs, and also the extent of those responses, or that a patient will survive, following surgical removal of the primary tumor and/or chemotherapy for a certain period of time without cancer recurrence. The methods of the present invention can be used clinically to make treatment decisions by choosing the most appropriate treatment modalities for any particular patient. The methods of the present invention are tools in predicting if a patient is likely to respond favorably to a treatment regimen, such as surgical intervention, chemotherapy with a given drug or drug combination, and/or radiation therapy, or whether long-term survival of the patient without cancer recurrence is likely, following surgery and/or termination of chemotherapy or other treatment modalities.

The term “breast cancer prognostic biomarker” refers to an RNA transcript, or an expression product thereof, intronic RNA, lincRNA, intergenic sequence, and/or intergenic region found to be associated with long term survival without breast cancer recurrence as disclosed herein.

The term “reference” RNA transcript or an expression product thereof, as used herein, refers to an RNA transcript or an expression product thereof, whose level can be used to compare the level of an RNA transcript or its expression product in a test sample. In an aspect of the invention, reference RNA transcripts include housekeeping genes, such as beta-globin, alcohol dehydrogenase, or any other RNA transcript, the level or expression of which does not vary de-pending on the disease status of the cell containing the RNA transcript or its expression product. In another aspect, all of the assayed RNA transcripts, or their expression products, or a subset thereof, may serve as reference RNA transcripts or reference RNA expression products.

As used herein, the term “RefSeq RNA” refers to an RNA that can be found in the Reference Sequence (RefSeq) database, a collection of publicly available nucleotide sequences and their protein products built by the National Center for Biotechnology Information (NCBI). The RefSeq database provides an annotated, non-redundant record for each natural biological molecule (i.e. DNA, RNA or protein) included in the database. Thus, a sequence of a RefSeq RNA is well-known and can be found in the RefSeq database at the Internet site: www (dot) ncbi (dot) nlm (dot) nih (dot) gov (slash) RefSeq (slash). See also Pruitt et al., Nucl. Acids Res. 33 (Supp 1): D501-D504 (2005). Accession numbers for each RefSeq, which include accession numbers for any alternative splice forms, are provided in Tables 1 and 2 and in Table B. The intronic sequences for a RefSeq are also publicly available. Nonetheless, the coordinates for each intronic sequence listed in Table 3 are provided in Table A. Therefore, the sequence of each RNA sequence in Tables 1-3 and 15 are readily available from publicly available sources.

As used herein, the term “RNA transcript” refers to the RNA transcription product of DNA and includes coding and non-coding RNA transcripts. RNA transcripts include, for example, mRNA, an unspliced RNA, a splice variant mRNA, a microRNA, fragmented RNA, long intergenic non-coding RNAs (lincRNAs), intergenic RNA sequences or regions, and intronic RNAs.

The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

The term “computer-based system,” as used herein, refers to the hardware means, software means, and data storage means used to analyze information. The minimum hardware of a patient computer-based system comprises a central processing unit (CPU), input means, output means, and data storage means. A skilled artisan can readily appreciate that many of the currently avail-able computer-based system are suitable for use in the present invention and may be programmed to perform the specific measurement and/or calculation functions of the present invention.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming and can be read by a suitable reader communicating with each processor at its corresponding station.

### II. Method of Extraction of Nucleic acids

In some aspects, the disclosure provides extraction of DNA and RNA from unstained FFPE material mounted on glass microscope slides that were stored with no particular special processing or storage conditions for over 30 years. In some aspects, specimens used for extraction can be cut from FFPE blocks 20 years old and stored with no special processing or storage conditions. Meticulous QA and QC assessments can be performed, along with advanced molecular profiling consisting of next generation sequencing (NGS) approaches to profile DNA mutations and copy number alterations. RNA-seq can be performed, and a custom droplet digital PCR (ddPCR) assay was further developed and optimized for archival specimens to determine the tumor molecular subtype.

In some aspects, the method of molecular subtyping a tumor sample obtained from a subject comprises a) enhanced solubilization of the old and degraded FFPE sample material through the non-discretionary use of mineral oil; b) digesting the sample with a proteinase; incubating the digested sample from step a) with a DNase; incubating the mixture from step b) with a guanidine salt based buffer; concentrating and isolating the RNA; pre-amplifying; and performing digital droplet PCR.

The methodology disclosed herein is described in greater detail below.

### (a) Sample

The disclosure encompasses a method of molecular subtyping a tumor in a sample comprising nucleic acids. The term encompasses tumor tissue samples, for example, tissue obtained by surgical resection and tissue obtained by biopsy, such as for example, a core biopsy or a fine needle biopsy. In a particular aspect, the tumor sample is a fixed, wax-embedded tissue sample, such as a formalin-fixed, paraffin-embedded tissue sample.

The method for fixing and embedding tissue samples are we known in the art. In some aspects the method comprises specimen fixation, dehydration, clearing, paraffin infiltration or impregnation, blocking or embedding in a block of paraffin, slicing the block and specimen into thin sections, mounting the sections on microscope slides, or any combinations thereof. In some aspects, the tissue samples can be fixed in a formalin solution (e.g., a 10% formalin solution may contain 3.7% formaldehyde and 1.0 to 1.5% methanol). The sample can then be dehydrated, e.g., by placing the sample in an alcohol, and then “cleared” of the alcohol by exposing the sample to a solvent such as xylene. The sample can then embedded in paraffin, where the sample is surrounded by paraffin which replaces the xylene in the sample. In some aspects, the fixed and embedded tissue samples are further stored. In some aspects, the storage is at room temperature. In some aspects, the samples are old and/or degraded.

In an aspect, the disclosure encompasses a method of identifying a molecular subtype in an old/aged sample or fresh sample comprising nucleic acid. Generally speaking, uncertainty about the fidelity of aged RNA samples remains a serious limitation. Aged tissue processing and sample storage are known to result in highly degraded RNA, which limits detection and introduces sequencing artifacts. However, the present disclosure provides methods for overcoming these limitations. The age of a sample can be determined based on the time that has passed since the sample was obtained from the subject. In some aspects, the old sample for use within the present methods may be at least 1 month old, at least 2 months old, at least 3 months old, at least 4 months old, at least 5 months old, at least 6 months old, at least 7 months old, at least 8 months old, at least 9 months old, at least 10 months old, at least 11 months old, at least 1 year old, at least 2 years old, at least 3 years old, at least 4 years old, at least 5 years old, at least 6 years old, at least 7 years old, at least 8 years old, at least 9 years old, at least 10 years old, at least 11 years old, at least 12 years old, at least 13 years old, at least 14 years old, at least 15 years old, at least 16 years old, at least 17 years old, at least 18 years old, at least 19 years old, at least 20 years old, at least 21 years old, at least 22 years old, at least 23 years old, at least 24 years old, at least 25 years old, at least 26 years old, at least 27 years old, at least 28 years old, at least 29 years old, at least 30 years old, at least 31 years old, at least 32 years old, at least 33 years old, at least 34 years old, at least 35 years old, at least 36 years old, at least 37 years old, at least 38 years old, at least 39 years old, at least 40 years old, at least 41 years old, at least 42 years old, at least 43 years old, at least 44 years old, at least 45 years old, at least 46 years old, at least 47 years old, at least 48 years old, at least 49 years old, at least 50 years old or greater than 50 years old.

In some aspects, the disclosure encompasses a method of identifying a molecular subtype in a degraded sample. In some aspects, the degraded sample is a FFPE sample. In some aspects, the method comprises identifying a molecular subtype in an old and degraded sample. In some aspects, the old and degraded sample is a FFPE. In some aspects, the FFPE sample for use within the present methods may be at least 1 month old, at least 2 months old, at least 3 months old, at least 4 months old, at least 5 months old, at least 6 months old, at least 7 months old, at least 8 months old, at least 9 months old, at least 10 months old, at least 11 months old, at least 1 year old, at least 2 years old, at least 3 years old, at least 4 years old, at least 5 years old, at least 6 years old, at least 7 years old, at least 8 years old, at least 9 years old, at least 10 years old, at least 11 years old, at least 12 years old, at least 13 years old, at least 14 years old, at least 15 years old, at least 16 years old, at least 17 years old, at least 18 years old, at least 19 years old, at least 20 years old, at least 21 years old, at least 22 years old, at least 23 years old, at least 24 years old, at least 25 years old, at least 26 years old, at least 27 years old, at least 28 years old, at least 29 years old, at least 30 years old, at least 31 years old, at least 32 years old, at least 33 years old, at least 34 years old, at least 35 years old, at least 36 years old, at least 37 years old, at least 38 years old, at least 39 years old, at least 40 years old, at least 41 years old, at least 42 years old, at least 43 years old, at least 44 years old, at least 45 years old, at least 46 years old, at least 47 years old, at least 48 years old, at least 49 years old, at least 50 years old or greater than 50 years old.

In some aspects, the sample is a tissue sample, a cell sample, a whole blood sample, a plasma sample, a serum sample, or a combination thereof. In some aspects, the tissue sample is a cancer or tumor tissue sample. In some aspects, the cancer or tumor tissue sample comprises cancer or tumor cells, tumor-infiltrating immune cells, stromal cells, or a combination thereof. In some aspects, the sample is a fixed sample. In some aspects, the fixed sample is fixed with a compound selected from the group consisting of formalin, glutaraldehyde, alcohol, osmic acid, and paraformaldehyde. In some aspects, the sample is paraffin-embedded. In some aspects, the sample is a formalin fixed paraffin-embedded sample selected from the group consisting of a fine needle aspirate (FNA), a core biopsy, and a needle biopsy. In some aspects, the cancer or tumor tissue sample is a formalin-fixed and paraffin-embedded (FFPE) sample, an archival sample, a fresh sample, or a frozen sample. In some aspects, the cancer or tumor tissue sample is a FFPE sample.

In some aspects, the cancer is selected from the group consisting of a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy. In some aspects, the cancer is a lung cancer, a kidney cancer, a bladder cancer, or a breast cancer. In some aspects, the lung cancer is a non-small cell lung cancer (NSCLC). In some aspects, the kidney cancer is a renal cell carcinoma (RCC). In some aspects, the bladder cancer is a urothelial bladder cancer (UBC). In some aspects, the breast cancer is a triple negative breast cancer (TNBC).

### (b) Nucleic Acid Extraction

In an aspect, a method of the disclosure comprises, in part, methods for extracting nucleic acid from a formalin-fixed and paraffin-embedded (FFPE) sample which may be old and or degraded. Often, for example with archival FFPE samples, the tissue is mounted on a glass slide. Prior to further processing a slide mounted FFPE sample there is a mandatory use of organic solvent that provides enhanced sample solubilization and ultimately improves the release and overall yield of nucleic acids. In some aspects, the organic solvent is xylene, CitriSolv, or mineral oil. In some aspects, the organic solvent is mineral oil. In some aspects, the mineral oil is a light mineral oil.

In some aspects, the methods include extracting nucleic acid from the FFPE sample comprising a first heating step where the sample is heated in the presence of mineral oil. In one aspect, the sample is heated at a temperature between about 65° C. and about 95° C. In an aspect, the sample is heated at a temperature of about 80° C. In one aspect, the first heating step can be from about 5 minutes to about 15 minutes. In certain aspects, the first heating step is about 10 minutes.

In some aspects, the method for extracting nucleic acid from FFPE sample further comprises digesting the sample with a proteinase. In some aspects, the protease is a proteinase K or trypsin. In some aspects, the protease is a proteinase K. In some aspects, the sample and proteinase can be incubated for a period of time e.g., between 15 minutes to 1 hour, or over an hour). In some aspects, incubation of a sample with proteinase occurs for approximately 1 hour. In some aspects, incubation of a sample with proteinase occurs for approximately 15 minutes. In some aspects, incubation of a sample with proteinase occurs for approximately 30 minutes.

After the first heating step the sample can be in digested in a suitable digestion buffer such as, in a non-limiting example, Proteinase K Digestion Buffer (e.g., sold commercially by Qiagen). The sample can be incubated in the digestion buffer for a suitable time to allow for DNA solubilization and liberation. Since much of DNA is wrapped around histone proteins, the enhanced solubilization allows for improved sample purification and bolsters activity by polymerases at downstream steps. In one aspect, the sample is digestion step includes incubating that sample in digestion buffer at a temperature from about 55° C. to about 80° C. optionally with constant agitation of the buffer and sample. In a preferred aspect, the sample is digested between about 65° C. to about 70° C. for about 45 minutes. After about 45 minutes the digestion buffer and sample are heated between about 60° C. and about 100° C. for about 15 minutes and then allowed to separate into an upper phase and lower phase using centrifugation. In some aspects, additional Proteinase K is added into the lower phase and the Proteinase K and sample are incubated optionally with agitation at a temperature from about 55° C. to about 80° C. for about 30 minutes.

After the sample is digested with the PKD buffer the sample can be centrifuged and the aqueous phase (bottom layer) separated from the residual lysate. In some aspects, in addition to protease treatment, the sample may be subject to RNase digestion, to remove residual RNA, and conversely, when processing a sample of RNA, the sample may be subject to DNase digestion, to remove residual DNA. In some aspects, a DNase is added to the aqueous phase and allowed to incubate for period of time. In some aspects, incubation of the sample may be done for a suitable period, e.g., extended for 1 hour or more or for less than 1 hour (e.g., 15 minutes). In some aspects, the sample is incubated with DNAse for about 15 minutes.

In some aspects, Buffer RBC is then added to the aqueous phase-DNase mixture and mixed prior to the addition of ethanol. In further aspects, RNA is purified and concentrated using, for example, RNeasy MinElute spin columns. The concentrated and purified RNA is then eluted using RNase-free water. Suitable equivalent buffers are understood by the skilled artisan useful within the methods of the disclosure. For example, RBC buffer based on guanidine hydrochloride (guanidine salt-based buffer). It is used to adjust RNA binding conditions, alternative buffers could be “home brewed” based on guanidine salts. These type of approaches (nucleic acid/RNA binding conditions, etc) are discussed in Molecular Cloning—Lab Manual (aka Maniatis manual) and incorporated herein by reference. As another example, other alcohols may be used, for instance EtOH vs isopropanol vs combination-based approaches. For instance, the Qiagen All-Prep kit uses isopropanol first to assist with RNA isolation, then EtOH at a later step for DNA. So, other alcohols may be used and “sequenced or introduced in an ordered manner” depending on the goal or aim in the protocol, at that particular juncture.

In a specific aspect, the protocol to extract RNA from an FFPE sample is as follows:

### Prep Stage:

1. Set the rotisserie oven to 65-70° C.
2. Set the heating block to 80° C.
3. Clean the razor blades with EtOH. One razor blade per sample is needed.
   Day 1:
4. Apply 10 μl of light mineral oil to wet the tissue. Note: the use of mineral oil or equivalent at this step and throughout this protocol is non-discretionary.
5. Scrape the desired tissue from the slide into the 2 ml screw cap tube (Note: sometimes you can see a lot of paraffin around the tissue, try to avoid scraping unnecessary paraffin).
6. Save slides for future reference.

### RNA Extraction:

7. Add 0.8 ml of mineral oil to the sample tube.
8. Heat the samples at the heating block (80° C.) for 10 min.
9. Remove the tubes from the heating block and quick spin.
10. Add 360 μl of Buffer PKD.
11. Add 40 μl of Qiagen Proteinase K.
12. Incubate at the rotisserie oven set to 65-70° C. for 45 min.
13. Incubate at the heating block (80° C.) for 15 min.
14. Quick spin.
15. Add 25 μl Qiagen Proteinase K into the lower phase.
16. Incubate at the rotisserie oven set to 65-70° C. for 30 min.
17. Begin thawing the DNase I on ice for later step.
18. Centrifuge the samples at max speed (13K rpm) for 15 min.
19. For each sample set up and label: two 2 ml tubes, one Qiagen RNeasy mini elute tube from the FFPE kit (stored at 4° C.), and one 1.5 ml elution tube.
20. After the centrifugation, transfer 250 μl of aqueous phase (bottom layer) into a new 2 ml labeled tube taking care not to disturb the pellet or aspirate the mineral oil. Set aside the residual lysate tube for later DNA extraction.
21. To the 250 μl aqueous phase add 25 μl DNase Booster buffer and 10 μl DNase I solution. Mix by inverting the tube.
22. Quick spin.
23. Incubate at RT for 15 min. (If doing the DNA extraction the next day, set up the overnight incubation of the residual lysates from step 13. Add 120 ul ATL and 30 μl of proteinase K to each sample and make sure the caps on the tubes are screwed tightly. Place in the rotisserie oven set to 65-70° C. for overnight incubation).
24. Quick spin.
25. Add 500 μl Buffer RBC (from RNeasy FFPE kit) and mix by vortexing.
26. Divide the sample into two 2 ml tubes with 392.5 μl in each tube.
27. Add 875 μl of 100% EtOH into each tube and mix well by pipetting up and down. Proceed immediately to the next step.
28. Transfer 700 μl of the sample into labeled RNeasy MiniElute spin column and centrifuge for 15 sec.
29. Discard the flow-through and repeat the previous step until the entire sample has passed through the column.
30. Add 500 μl buffer RPE to the column and centrifuge at max speed for 15 sec.
31. Add 500 μl buffer RPE to the column and centrifuge at max speed for 1 min.
32. Discard the flow-through, open the lid of the spin column and centrifuge at max speed for 5 min.
33. Discard the flow-through and place the spin column into a labeled 1.5 ml elution tube.
34. Add 30 μl RNase-free water directly to the column membrane and incubate at RT for 1 min.
35. Centrifuge at max speed for 1 min to elute the RNA. Final eluted volume should be 30 μl.
36. Check the RNA concentration on Qubit.

In another aspect, the methods include extracting DNA from the FFPE sample comprising incubating the residual lysate, as described above, with ATL and proteinase K buffer at a temperature from about 55° C. to about 80° C., optionally with constant agitation, for about 11 to about 15 hours. Buffer ATL is A Tissue Lysis buffer used in the purification of nucleic acids. Buffer ATL 0 Buffer comprising EDTA and SDS sodium dodecyl sulfate (SDS), which is an anionic surfactant (detergent) that helps with tissue lysis, by disrupting non-covalent bonds in proteins which aids in the overall denaturing process, important for the liberation of nucleic acids. It is also noted, this protocol is not dependent on particular columns (e.g. Qiagen), as a modified method using “bead-based” methods for molecular separation steps has been successfully implemented.

In some aspects, additional proteinase K buffer can be added during the incubation time to ensure the tissue is completely digested. After the tissue is completely digested RNase A can be added with binding buffer PM and sodium acetate. Buffer PM is a molecular biology binding buffer comprising guanidinium chloride and 2-propanol. Buffer PM replacement solution of the present invention 64% by volume tetraethylene glycol; 24% by volume ethanol; 100 mM. NaCl; 10 mM Tris pH 7.5. The DNA within the mixture can then be isolated and concentrated using, for example, a spin column. The DNA may then be eluted using heated elution buffer.

In a specific aspect, the protocol to extract DNA from an FFPE sample is as follows:
1. Add 120 μl ATL and 30 μl of proteinase K to each sample of residual lysate (obtained from the RNA extraction protocol) and make sure the caps on the tubes are screwed tightly. Place in the rotisserie oven set to 65-70° C. for overnight incubation.
2. Add 20 μl proteinase K to the aqueous phase and pipet up and down.
3. Return to the rotisserie oven set to 65-70° C. for additional 1-2 hours to ensure that the tissue is completely digested.
4. Repeat step 2-3 if unlysed tissue remains.
5. While waiting, for each sample label one 1.5 ml Eppendorf tubes for mixing binding buffer, one Qiaquick spin column, one collection tube and one 1.5 ml tubes for elution. Prepare 80% EtOH solution. Set the heat block to 65° C.
6. Quickspin.
7. Add 1.5 μl RNase A and vortex for 3-5 seconds, quickspin and incubate at room temperature for 5 min.
8. Into a labeled 1.5 ml Eppendorf tubes add 490 μl binding buffer PM and 10 μl 3M sodium acetate.
9. Into that tube add around 250 μl of aqueous phase, bottom layer (first, find out how much aqueous phase is there. Then, pipette 200 μl first, then pipet the rest slowly, avoid getting any oil in the sample). Mix the sample with buffer by pipetting up and down. Save the remaining lysed tissue in 2 ml tubes indefinitely, until the presence of the DNA in the eluted sample is verified.
10. Apply 700 μl of the sample into a labeled a Qiaquick spin column and centrifuge at 2,000 rpm for 90 sec.
11. Since not all the DNA may bind to the column, we need to re-apply the flow-through, so save the flow through and labeled collection tube.
12. Place the spin column into a new collection tube and apply the rest of the sample from step 9 and centrifuge at 2,000 rpm for 90 sec.
13. Place the spin column into a fresh collection tube and apply the flow-through from step 11 and centrifuge at 2,000 rpm for 90 sec. Do the same with the remaining flow-through from step 12.
14. Add 700 μl of buffer PE (this is a wash buffer with a weak organic base
15. 10 nM Tris-HCL pH 7.5, 80% EtOH) and centrifuge at 10,000 rpm for 15 sec. Discard the flow-through.
16. Add 700 μl of 80% EtOH and centrifuge at max speed for 1 min.
17. Discard the flow-through and centrifuge at max speed for 5 min.
   (c) Preparation of Preamplification Reaction cDNA Product and ddPCR

PCR-based preamplification is a method used to increase the concentration of a specific panel of targets in a sample prior to qPCR analysis, reducing the required sample input for multi-target qPCR experiments. Preamplification is essentially a highly multiplexed PCR reaction performed for a limited number of cycles using the same primer sets that will be used in the downstream qPCR reaction. By using a reagent designed for preamplification with a limited number of PCR cycles, optimal amplification efficiency can be maintained for each target, which is essential to preventing the introduction of bias into the qPCR analysis. With just 10-14 cycles of preamplification, the concentration of each target is boosted by 1000-fold or more, providing enough pre-amplified sample to analyze all of the targets by qPCR without compromising the sensitivity of the qPCR analysis. As a rule of thumb, preamplification is useful any time the amount of sample available limits the number of targets that can effectively be analyzed. Pre-amplification can be done using methods know in the art. In an exemplary aspect, TaqMan preamp Supermix (ThermoFisher) can be used. In another exemplary aspect, SsoAdvandced PreAmp Supermix (Bio-Rad) can be used.

In general, amplification of the region of interest is carried out using polymerase chain reaction (PCR). A PCR reaction may comprise sample comprising nucleic acid, one or more primer pairs, polymerase, water, buffer, and deoxynucleotide triphosphates (dNTPs) in a single reaction vial. PCR may be performed according to standard methods in the art. By way of non-limiting example, the PCR reaction may comprise denaturation, followed by about 15 to about 30 cycles of denaturation, annealing and extension, followed by a final extension. In an exemplary aspect, the PCR reaction comprises denaturation at about 98° C. for about 30 seconds, followed by about 15 to about 30 cycles of (about 98° C. for about 10 seconds, about 62-72° C. for about 30 seconds, about 72° C. for about 30 seconds), followed by a final extension at about 72° C. for about 2 minutes.

In certain aspects, Droplet Digital PCR (ddPCR) is used. ddPCR is a method for performing digital PCR that is based on water-oil emulsion droplet technology. A sample is fractionated into 20,000 droplets, and PCR amplification of the template molecules occurs in each individual droplet. ddPCR technology uses reagents and workflows similar to those used for most standard TaqMan probe-based assays. The massive sample partitioning is a key aspect of the ddPCR technique. Droplets are formed in a water-oil emulsion to form the partitions that separate the template DNA molecules. The droplets serve essentially the same function as individual test tubes or wells in a plate in which the PCR reaction takes place, albeit in a much smaller format. The massive sample partitioning is a key aspect of the ddPCR technique. The Droplet Digital PCR System partitions nucleic acid samples into thousands of nanoliter-sized droplets, and PCR amplification is carried out within each droplet. This technique has a smaller sample requirement than other commercially available digital PCR systems, reducing cost and preserving precious samples.

In some aspects, one or more droplets are formed, each containing a nucleic acid and a heterogeneous mixture of primer pairs and probes, each specific for multiple target sites on the template. For example, a first fluid (either continuous, or discontinuous as in droplets) containing a single nucleic acid template (DNA or RNA) is merged with a second fluid (also either continuous, or discontinuous as in droplets) containing a plurality of primer pairs and a plurality of probes, each specific for multiple targets sites on the nucleic acid template to form a droplet containing the single nucleic acid template and a heterogeneous mixture of primer pairs and probes. The second fluid can also contain reagents for conducting a PCR reaction, such as a polymerase and dNTPs.

In some aspects, certain members of the plurality of probes include a detectable label. Members of the plurality of probes can each include the same detectable label, or a different detectable label. The detectable label is preferably a fluorescent label. The plurality of probes can include one or more groups of probes at varying concentrations. The one or more groups of probes can include the same detectable label which varies in intensity upon detection, due to the varying probe concentrations.

In some aspects, the first and second fluids can each be in droplet form. Any technique known in the art for forming droplets may be used with methods of the invention. An exemplary method involves flowing a stream of the sample fluid containing the nucleic acid such that it intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets containing the first fluid. The carrier fluid may be any fluid that is immiscible with the sample fluid. An exemplary carrier fluid is oil. In certain aspects, the carrier fluid includes a surfactant, such as a fluorosurfactant. The same method may be applied to create individual droplets from the second fluid containing the primer pairs (and, in some implementations, the amplification reagents). Either the droplets containing the first fluid, the droplets containing the second fluid, or both, may be formed and then stored in a library for later merging.

In some aspects, the nucleic acid in each of the merged/formed droplets is amplified, e.g., by thermocycling the droplets under temperatures/conditions sufficient to conduct a PCR reaction. The resulting amplicons in the droplets can then be analyzed. In some aspects, the method further comprises digital PCR. In some aspects, droplet containing the nucleic acid can be merged with the PCR reagents in the second fluid as described above, producing a droplet that includes Taq polymerase, deoxynucleotides of type A, C, G and T, magnesium chloride, forward and reverse primers, detectably labeled probes, and the target nucleic acid. In another aspect, the first fluid can contain the template DNA and PCR master mix (defined below), and the second fluid can contain the forward and reverse primers and the probe. The disclosure is not restricted in any way regarding the constituency of the first and second fluidics for PCR or digital PCR. For example, in some aspects, the template DNA is contained in the second fluid inside droplets. In some aspects, multiplexed primer pairs can be used in the droplet-based digital PCR reaction.

However, the method of the present disclosure is not particularly limited to a specific PCR detection method. It is noted that other PCR methods than ddPCR (e.g. qPCR) are useful in the method steps disclosed herein.

In other aspects, the extracted nucleic acid is subjected to other processing. In some aspects, the extracted nucleic acid is subjected to molecular profiling. For e.g., commercially available Illumina RNA Access or custom NanoString nCounter assay can be used for molecular profiling of extracted nucleic acid. In some aspects, extracted nucleic acid can be used to build a nucleic acid library. In some aspects, the extracted nucleic acid is subjected to sequencing, for e.g., whole transcriptome RNA-seq, or Next generation sequencing (NGS).

In further aspect, the extracted nucleic acid is subjected to molecular target quantitation and subtype determination.

### III. Methods of Use

The method of the disclosure can be further used to quantitate, determine a sequence and/or determine the cancer subtype using the extracted nucleic acids from the sample.

In some aspects, the abundance of two or more target or reference nucleic acid may be compared. In some aspects, the target nucleic acid comprise one or more hormonal targets, human epidermal growth factor receptor 2 (HER2) targets and proliferation targets. In some aspects, the hormonal target comprise nucleic acid, or fragments thereof, encoding, Estrogen receptor 1 (ESR1), Progesterone receptor (PGR), B-cell lymphoma 2 (BCL2), Signal Peptide, CUB Domain And EGF Like Domain Containing 2 (SCUBE2), or any combinations thereof. In some aspects, HER2 targets comprise nucleic acid or fragments thereof encoding human epidermal growth factor receptor 2 (HER2), Growth factor receptor-bound protein 7 (GRB7), or any combination thereof. In some aspects, the proliferation targets comprise nucleic acid or fragments thereof encoding Marker Of Proliferation Ki-67 (MKI67), Aurora kinase A (AURKA), Baculoviral IAP Repeat Containing 5 (BIRC5), Cyclin B1 (CCNB1), MYB Proto-Oncogene Like 2 (MYBL2), Thymidine kinase 1 (TK1), or any combination thereof.

In some aspects, the method of the disclosure comprises determining the level of ESR1, PGR, BCL2, SCUBE2, HER2, GRB7, MK167, AURKA, BIRC5, CCNB1, MYBL2, TK1, or any combination thereof. In some aspects, the disclosed method comprises determining the level of ESR1, PGR, BCL2, SCUBE2, or any combination thereof. In some aspects, the disclosed method comprises determining the relative level of HER2, GRB7, or any combination thereof. In some aspects, the disclosed method comprises determining the level of MKI67, AURKA, BIRC5, CCNB1, MYBL2, TK1, or any combination thereof. In some aspects, the disclosed method comprises determining the level of ESR1, PGR, BCL2, SCUBE2, HER2, GRB7, MK167, AURKA, BIRC5, CCNB1, MYBL2, and TK1.

The level of target nucleic acid can be determined using the method described in Section II, for e.g., ddPCR. However, any known method in the art can be used for determining the levels of target nucleic acids. By way of non-limiting examples, levels of the levels of target nucleic acids can be measured using RNA-seq, nanopore sequencing, Nanostring, multiplex RT-PCR, single-plex RT-PCR, NASBA, Fluorescence measurements or spectrophotometry.

In further aspects of the disclosure, the method comprises determining z-score of the target nucleic acids. The z-score can be determined by using the following equation:

$${x}^{′} = ( x - \overline{x} ) / SD$$

where x is the observed value, x̅ is the mean and SD is the standard deviation of the targets within that particular sample.

In some aspects, the target nucleic acid is considered having increased levels if the z-score is ≥1. In some aspects, a target nucleic acid having increased level comprises a z-score from about 1 to about 5. For example, the z-score can be about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

In some aspects, the target nucleic acid is considered having decreased levels if the z-score is <1. In some aspects, a target nucleic acid having decreased level comprises a z-score from about −0.1 to about −5. For example, z-score can be about −0.1, −0.2, −0.3, −0.4, −0.5, −0.6, −0.7, −0.8, −0.9, −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, −3.9, −4, −4.1, −4.2, −4.3, −4.4, −4.5, −4.6, −4.7, −4.8, −4.9, or −5.

In further aspect, the disclosed method encompasses determining the cancer subtype of the sample using z-score of target nucleic acids. In some aspects, the cancer subtype determined using the disclosed method comprise Luminal A subtype (Lum A), Luminal B subtype (Lum B), HER2 subtype or Triple Negative subtype (TN).

In some aspects, a sample is determined to be Lum A if the level of one or more of the hormonal target nucleic acid or fragment thereof is determined to be elevated. In some aspects, the sample is determined to be Lum A if the level of nucleic acid or fragment thereof encoding ESR1, PGR, BCL2, SCUBE2, or any combination thereof, is elevated. In some aspects, the sample is determined to be Lum A if the level of nucleic acid or fragment thereof encoding ESR1 is elevated. In some aspects, the sample is determined to be Lum A if the level of nucleic acid or fragment thereof encoding PGR is elevated. In some aspects, the sample is determined to be Lum A if the level of nucleic acid or fragment thereof encoding BCL2 is elevated. In some aspects, the sample is determined to be Lum A if the level of nucleic acid or fragment thereof encoding SCUBE2 is elevated. In some aspects, the sample is determined to be Lum A if the level of nucleic acid or fragment thereof encoding ESR1, PGR, BCL2, and SCUBE2 are elevated.

In some aspects, a sample is determined to be Lum B if the level of one or more of the hormonal target nucleic acid or fragment thereof and one or more proliferation target nucleic acid or fragment thereof are determined to be elevated. In some aspects, the sample is determined to be Lum B if the level of nucleic acid or fragment thereof encoding ESR1, PGR, BCL2, SCUBE2, or any combination thereof, is elevated, and if the level of nucleic acid or fragment thereof encoding MKI67, AURKA, BIRC5, CCNB1, MYBL2, TK1, or any combination thereof is elevated or combinations of any combination thereof. In some aspects, the sample is determined to be Lum B if the level of nucleic acid or fragment thereof encoding one or more of ESR1, PGR, BCL2, and SCUBE2 is elevated, and if the level of nucleic acid or fragment thereof encoding one or more of MKI67, AURKA, BIRC5, CCNB1, MYBL2 and TK1 is elevated.

In some aspects, a sample is determined to be HER2 if the level of one or more of the HER target nucleic acid or fragment thereof is determined to be elevated. In some aspects, the sample is determined to be HER2 if the level of nucleic acid or fragment thereof encoding HER2, GRB7, or any combination thereof is elevated. In some aspects, the sample is determined to be HER2 if the level of nucleic acid or fragment thereof encoding HER2 is elevated. In some aspects, the sample is determined to be HER2 if the level of nucleic acid or fragment thereof encoding GRB7 is elevated. In some aspects, the sample is determined to be HER2 if the level of nucleic acid or fragment thereof encoding HER2 and GRB7 are elevated.

In some aspects, a sample is determined to be TN if the level of one or more of the proliferation target nucleic acid or fragment thereof is determined to be elevated. In some aspects, the sample is determined to be TN if the level of nucleic acid or fragment thereof encoding MK167, AURKA, BIRC5, CCNB1, MYBL2, TK1, or any combination thereof is elevated. In some aspects, the sample is determined to be TN if the level of nucleic acid or fragment thereof encoding MK167 is elevated. In some aspects, the sample is determined to be TN if the level of nucleic acid or fragment thereof encoding AURKA is elevated. In some aspects, the sample is determined to be TN if the level of nucleic acid or fragment thereof encoding BIRC5 is elevated. In some aspects, the sample is determined to be TN if the level of nucleic acid or fragment thereof encoding CCNB1 is elevated. In some aspects, the sample is determined to be TN if the level of nucleic acid or fragment thereof encoding MYBL2 is elevated. In some aspects, the sample is determined to be TN if the level of nucleic acid or fragment thereof encoding TK1 is elevated. In some aspects, the sample is determined to be TN if the level of nucleic acid or fragment thereof encoding MKI67, AURKA, BIRC5, CCNB1, MYBL2, and TK1 are elevated.

In some aspects, the disclosed method provides high sensitivity, accuracy, and/or reproducibility of cancer subtype determination of an old and/or degraded sample. In some aspects, the sensitivity, accuracy, and/or reproducibility of cancer subtype determination is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least 100% higher as compared to other methods for determining cancer subtype, for e.g., Immunohistochemistry (IHC), biopsy test, determination using the IntClust algorithm, PAM50 assay etc.

In further aspects, the disclosed method encompasses verification of quantification of the target nucleic acids and/or, the cancer subtype determination. In some aspects, methods including distance geometry studies can be applied for verification. In some aspects, the subtype determination can be confirmed using assistance from a qualified pathologist.

The method of the disclosure further comprises determining the genomic characteristics of the extracted nucleic acids. The disclosure further encompasses sequencing analysis of the extracted nucleic acids. In some aspects, the sequencing of extracted nucleic acids is carried out using commercially available sequencing technology SBS (sequencing by synthesis) by Ulumina, chain termination method of DNA sequencing, one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing. In some aspects, the sequencing analysis can comprise NGS, and/or low-pass/ultra-low-pass WGS. Methods related to NGS, and WGS are well known in the art.

In further aspects, chromosome copy number alterations (CNA), and/or mutations of target genes can be assessed after sequencing of nucleic acids. In one aspect, the CNA can be used to determine the tumor fraction of a sample. In another aspect, the CNA can be used to determine the cancer subtype. In some aspects, CNA is used for diagnosing cancer in a subject.

In some aspects, determination of CNA comprises isolating DNA from a biological sample using disclosed method, sequencing the DNA using ULP-WGS, analyzing the sequence of the DNA using statistical models (for e.g., using ichorCNA), and characterizing the copy number alterations present in the sample, for example, by generating a copy number alteration profile.

In some aspects, determination of mutations in target genes include creating a library (for e.g., using The QIAGEN QIAseq Human Breast Cancer Panel (DHS-001Z, 93 Genes) library prep kit) using nucleic acids extracted using method described herein, sequencing using NGS (e.g., Illumina HiSeq 3000 with 150 bp PE) and performing bioinformatic analyses (for. e.g., Qiagen, smCounter2-based bioinformatic analyses). In some aspects, the method of the disclosure further encompasses mutational analysis of the target gene. The mutational analysis can further comprise determining the mutational effect on protein function, as high effect, as moderate effect, as low effect, and/or as no mutation.

In one aspect, target genes comprise a panel of genes. In some aspects, the gene comprise one or more genes disclosed in Tables 25-53. In some aspects, the genes comprise Adenomatous polyposis coli (APC), Ataxia-telangiectasia mutated (ATM), ataxia telangiectasia and Rad3-related protein (ATR), BRCA1 Associated Ring Domain 1 (BARD1), BLM RecQ Like Helicase (BLM), Breast cancer type 1 (BRAC1), Breast cancer type 2 (BRAC2), CUB And Sushi Multiple Domains 1 (CSMD1), Epidermal growth factor receptor (EGFR), Erb-B2 Receptor Tyrosine Kinase 3 (ERBB3), Fibroblast growth factor receptor 2 (FGFR2), GEN1 Holliday Junction 5′ Flap Endonuclease (GEN1), HECT And RLD Domain Containing E3 Ubiquitin Protein Ligase Family Member 1 (HERC1), Lysine Methyltransferase 2C (KMT2C), Mitogen-Activated Protein Kinase Kinase Kinase 1 (MAP3K1), DNA mismatch repair protein Mlh1 (MLH1), MRE11 Homolog, Double Strand Break Repair Nuclease (MRE11A), Mucin 16 (MUC16), Nuclear Receptor Corepressor 1 (NCOR1), Neurofibromatosis type 1 (NF1), Palladin, Cytoskeletal Associated Protein (PALLD), phosphatidylinositol-4,5-bisphosphate 3-kinase, catalytic subunit alpha (PIK3CA), PMS1 homolog 2, mismatch repair system component (PMS2), Ret proto-oncogene (RET), Septin 9 (SEPT9), Spectrin repeat containing nuclear envelope protein 1 (SYNE1), Tumor protein P53 (TP53), or any combination thereof.

In some aspects, the target gene comprises APC. In some aspects, the target gene comprises ATM. In some aspects, the target gene comprises ATR. In some aspects, the target gene comprises BARD1. In some aspects, the target gene comprises BLM. In some aspects, the target gene comprises BRAC1. In some aspects, the target gene comprises BRAC2. In some aspects, the target gene comprises CSMD1. In some aspects, the target gene comprises EGFR. In some aspects, the target gene comprises ERBB3. In some aspects, the target gene comprises FGFR2, GEN1. In some aspects, the target gene comprises HERC1. In some aspects, the target gene comprises KMT2C. In some aspects, the target gene comprises MAP3K1. In some aspects, the target gene comprises MLH1. In some aspects, the target gene comprises MRE11A. In some aspects, the target gene comprises MUC16. In some aspects, the target gene comprises NCOR1. In some aspects, the target gene comprises NF1. In some aspects, the target gene comprises PALLD. In some aspects, the target gene comprises PIK3CA. In some aspects, the target gene comprises PMS2. In some aspects, the target gene comprises RET. In some aspects, the target gene comprises SEPT9. In some aspects, the target gene comprises SYNE1. In some aspects, the target gene comprises TP53. In some aspects, the target gene comprises APC, ATM, ATR, BARD1, BLM, BRAC1, BRAC2, CSMD1, EGFR, ERBB3, FGFR2, GEN1, HERC1, KMT2C, MAP3K1, MLH1, MRE11A, MUC16, NCOR1, NF1, PALLD, PIK3CA, PMS2, RET, SEPT9, SYNE1, TP53, or any combination thereof. In some aspects, the target gene comprises APC, ATM, ATR, BARD1, BLM, BRAC1, BRAC2, CSMD1, EGFR, ERBB3, FGFR2, GEN1, HERC1, KMT2C, MAP3K1, MLH1, MRE11A, MUC16, NCOR1, NF1, PALLD, PIK3CA, PMS2, RET, SEPT9, SYNE1, and TP53.

In one aspect, the mutation analysis of the target gene can be used to determine the tumor fraction of a sample. In another aspect, the mutation analysis of the target gene can be used to determine the cancer subtype. In some aspects, mutation analysis of the target gene is used for diagnosing cancer in a subject.

The method of the present disclosure is not particularly limited to a specific cancer. Subtypes of any cancer can be determined by adapting the disclosed method and selecting specific cancer associated target nucleic acids.

A method of the disclosure may be used to diagnose, treat or prevent a disease in a subject. Identification of a cancer subtype could facilitate the diagnosis of a disease, enable the proper methodology, such as a therapeutic, to treat the disease, or prevent the onset of disease by administration of prophylactic therapies. In an aspect, the disclosed methods can be used for guiding treatment of cancer and can comprise modifying the treatments, based on the analysis of the samples. For example, modifying treatment in an aspect, comprise changing the amount of one or more of the therapeutics, changing the frequency of administration, or by changing the duration of time one or more of the therapeutics are administered to a subject.

Still further, a method of the disclosure may be used to determine the responsiveness to a therapeutic agent. With reference to archival samples where outcome data is known, the knowledge gained from the disclosed methods may be used to assess responsiveness of a therapeutic agent in a cancer subtype and guide treatment decisions. Further disclosed method may be used to assess the health of the subject relative to their cancer subtype and, and guide treatment decisions, based on knowledge gained from archival samples with outcome data known.

In further aspects, the disclosed methods can be used for monitoring the subject for adverse effects. With reference to archival samples where outcome data is known, the knowledge gained from the disclosed methods may be used to correlate cancer subtype and adverse effects. In an aspect, in the absence of adverse effects, a disclosed method can further comprise continuing to treat the subject. In an aspect, in the presence of adverse effects, a disclosed method can further comprise modifying the treating step. Methods of monitoring a subject's well-being can include both subjective and objective criteria (and are discussed supra). Such methods are known to the skilled person.

In some aspects, various aspects of the disclosed methods can be automated using computer software analytical programs. The present disclosure, thus further provides computer implemented methods of detecting, comparing, and analyzing patterns of expression or levels of extracted nucleic acids, in order to diagnose cancer, determine the subtype of a cancer, or determining the course of treatment in a subject. The analytical programs can be interfaced with, for example, programs that are part of an automated nucleic acid detection or quantification system so that data from the automated detection or quantification system can fed directly to the analytical programs. Computer implemented programs can be implemented to output, for example, the identity of nucleic acids in the sample and the degree of increase or decrease in the abundance of the nucleic acids. In further aspects, the computer implemented program can be engineered to output cancer subtype, based on analysis of the nucleic acids. The interface between the analytical programs may be direct or indirect. In some aspects, the programs of this disclosure can be designed to accept information on the detection or quantification of nucleic acids, are able to implement data analysis, and output cancer subtype assessments. In some aspects the programs of disclosure can further output diagnosis of cancer or treatment strategies.

### IV. Kit

The present disclosure also encompasses a kit for carrying out a method according to one or more of the aspects of the invention. In some aspects, the kt can comprise a container, organic solvents, proteinase K and/or buffer for lysis, solutions and/or devices for nucleic acid extraction, solutions and/or devices for nucleic acid purification, solutions and/or devices for nucleic acid amplification, a manual and/or description for carrying out the method, or any combination thereof.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Having described several aspects, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

As various changes could be made in the above-described materials and methods without departing from the scope of the invention, it is intended that all matter contained in the above description and in the examples given below, shall be interpreted as illustrative and not in a limiting sense.

### Examples

It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific aspects which are disclosed and still obtain a like or similar result without departing from the spirit and scope of the invention.

Through extensive testing on challenging samples and selection of probes, the present example provides a method for the molecular subtyping of tumors from archival tissue. Distance Geometry, Sample Based FICA—Normalized Data) shows the results of hierarchical clustering analysis (HCA) following the processing by the disclosed method of 31 samples obtained from FFPE archival breast tissue blocks. The tissue blocks were obtained from UAMS (20 year old samples) and SWOG (30 year old samples) along with two cell lines (MCF, MDA). Regarding the samples, there were 11 Triple Negative (TN), 8 Luminal A (Lum A), 3 Luminal B (Lum B), 6 normal lymph nodes (LN), 2 Her2 enriched (Her2), and one atypical ductal hyperplasia. Sample MCF was from the MCF-7 cell line (Lum B), and sample MDA is cell line MDA-MB-453 (TN with weak Her2). Probes used in the approach are listed in Table 1.

TABLE 1
Probe listing

| Gene Symbol | Manufacturer | Assay ID | Catalog No. |
|---|---|---|---|
| ESR1 | Thermo | Hs01046816_m1 | 4331182 |
| | | Hs00174860_m1 | 4331182 |
| | | Hs01046813_m1 | 4351372 |
| PGR | Thermo | Hs01556707_m1 | 4331182 |
| | | Hs01556702_m1 | 4331182 |
| | | Hs01556701_m1 | 4331182 |
| | | Hs04332988_m1 | 4426961 |
| | | Hs00612017_s1 | 4331182 |
| ERBB2 | Thermo | Hs01001594_m1 | 4351372 |
| MKI67 | Thermo | Hs01032437_m1 | 4331182 |
| | | Hs01032443_m1 | 4331182 |
| | | Hs04981441_m1 | 4351372 |
| | | Hs01032432_g1 | 4331182 |
| | | Hs00267195_m1 | 4331182 |
| | | Hs04260396_g1 | 4331182 |
| BIRC5 | Thermo | Hs04194392_s1 | 4331182 |
| | | Hs03063352_s1 | 4331182 |
| | | Hs00153353_m1 | 4331182 |
| | | Hs00977612_mH | 4331182 |
| CCNB1 | Thermo | Hs01030099_m1 | 4331182 |
| | | Hs01030098_g1 | 4351372 |
| | | Hs01030097_m1 | 4331182 |
| AURKA | Thermo | Hs01597773_mH | 4331182 |
| | | Hs00269212_m1 | 4331182 |
| | | Hs01590514_m1 | 4351372 |
| BCL2 | Thermo | Hs04986394_s1 | 4331182 |
| | | Hs00608023_m1 | 4331182 |
| SCUBE2 | Thermo | Hs00221277_m1 | 4331182 |
| | | Hs01012951_m1 | 4351372 |
| | | Hs01012957_m1 | 4351372 |
| GRB7 | Thermo | Hs00917999_g1 | 4331182 |
| | | Hs00918011_g1 | 4351372 |
| | | Hs00918005_g1 | 4351372 |
| | Thermo | Hs00942540_m1 | 4331182 |
| MYBL2 | | Hs00942547_m1 | 4351372 |
| | | Hs00942543_m1 | 4331182 |
| | Thermo | Hs01062125_m1 | 4331182 |
| TK1 | | Hs01062126_g1 | 4351372 |
| | | Hs01062123_m1 | 4331182 |

### Methods.

A. Protocol: Nucleic Acid Extraction from Archival Specimens

Listed below are materials and devices that can be used for performing the methods provided herein.
1. Specimen Tracking Worksheet
2. Tissue Micro Array (TMA) Needle punch with stylet
3. Razor blades, standard size
4. 2.0 mL screw top microtubes (Sarstedt 72.694.006)
5. LowBind microtubes
6. 2 mL collection tubes (Qiagen)
7. Light mineral oil NF (ie, Geritrex brand, pharmacy)
8. Xylene (ie, Sigma 247642)
9. Thermomixer (Eppendorf)
10. Microcentrifuge (Eppendorf)
11. NanoDrop Spectrophotometer
12. 100% EtOH
13. Nuclease-Free Water (Ambion AM9937)
14. ATL buffer (Qiagen Cat #19076)
15. Proteinase K solution* (Qiagen Cat #19133)
16. RNAseA (Qiagen Mat #1007885)
17. QIAQuick (purple) columns (Qiagen #1018215)
18. PM buffer (Qiagen Mat #1018139)
19. PE buffer (Qiagen Mat #1015207)
20. Clean and Concentrate kit (Zymo Research)
21. RNeasy FFPE Kit* (Qiagen Cat #73504)
22. Agilent Bioanalyzer 2100 System or equivalent (e.g., Agilent Fragment Analyzer)
23. Agilent RNA 6000 Nano Kit (Cat #5067-1511)
24. LabNet mini-Incubator with tube rotisserie
   *NOTE: A few general guidelines regarding reagents and columns. They generally expire one (1) year after the date received in the lab with the following exceptions: RNeasy FFPE Kit (9 months); Proteinase K solution and RNAseA (2 years); light mineral oil NF (as stamped on bottle by manufacturer). Recommend recording expiration dates on all component bottles/bags and verify expiration dates at each use.

The method disclosed below can be used to perform pre-lysis transfer of tissue into a tube.

### Part 1: Pre-Lysis Tissue Transfer to Tube

### FFPE Blocks

1. Eject one-to-three 0.6-1.0 mm needle core punch(es) from FFPE block into a labeled 2.0 mL screwcap tube.
2. Verify sample tracking worksheet information, and that tube label matches sample tracking worksheet.
Important: file FFPE block(s) in appropriate designated location.
3. Go to protocol section “Part 2”.
Unstained Tissue Section Slides without Cover Slip (USS)
1. Apply 10 μL light mineral oil to wet the tissue. Scrape desired tissue from slide using a razor blade and put into 2.0 mL screwcap tube. This is a mandatory step.
2. IMPORTANT: Save slides for future reference/use. DO NOT DISCARD SLIDES.
3. Proceed to Step 2 of the “FFPE Blocks” protocol.

For stained slides with cover slips, including cytology smears, following method can be used.

Stained Slides with Cover Slip (Including Cytology Smears)
1. Verify slide is labeled in pencil (any stickers or ink will be removed in this process).
2. Soak slides in xylenes overnight (over the weekend is even better) in fume hood. NOTE: must use real xylenes, not xylene substitute.
3. When loosened, carefully slide off cover slips. Typically, the tissue will adhere to the glass slide rather than the cover slip.
4. Soak de-cover slipped slides in xylenes another 20 minutes to remove residual glue/mounting medium.
Dip the slide(s) up and down 10 times. NOTE: must use real xylenes, not xylene substitute.
5. Soak slides in 100% EtOH×10 minutes.
6. Air dry slides completely (˜15 minutes room temperature)
7. To facilitate slide scraping, rehydrate tissue by pipetting 10 μL of light mineral oil onto slide.
8. Scrape tissue from slides into appropriately labeled 2.0 mL microcentrifuge tubes.
9. IMPORTANT: Save slides for future reference/use. DO NOT DISCARD SCRAPED SLIDES.
10. Proceed to Step 2 of the “Part 1 FFPE blocks” protocol.

The method disclosed below can be used for partial tissue lysis and RNA extraction.

### Part 2: Partial Tissue Lysis and RNA Extraction

### RNA Extraction Using Qiagen RNeasy FFPE Kit

1. Add 0.8 mL mineral oil to the sample tube.
2. Add 360 μL/40 μL mixture of Buffer PKD/Qiagen Proteinase K.
3. Incubate at 65° C. for 45 min (stat mode) to 16 hours (standard mode) in heated rotisserie.
4. Incubate at 80° C. for 15 min if stat mode.
5. Quick spin.
6. Add 25 μL Qiagen Proteinase K to lower phase.
7. Incubate at 65° C. for 30 min in rotisserie. (Begin thaw DNase I on ice for later step)
8. Centrifuge max speed for 15 min. (For each sample set up/label: one 2 mL tube, one Qiagen column, five collection tubes for steps 21-27, one 1.5 mL tube for elution)
9. Transfer 250 μL aqueous phase (from the ˜400 μL original, leaving ˜150 μL behind) into a new 2.0 mL labeled microcentrifuge tube taking care not to disturb the pellet or aspirate mineral oil. Set aside residual lysate tube for later DNA extraction.
10. To the 250 μL portion, add 25 μL DNase Booster Buffer and 10 μL DNase I solution, and mix by inverting the tube.
11. Centrifuge briefly to collect residual liquid from the sides of the tube.
12. Incubate at room temperature for 15 min. NOTE: During this 15 min incubation, continue incubation of the residual lysates from step 12 above for DNA extraction: see Part 3 below.
13. Quick spin.
14. Add 500 μL Buffer RBC and mix by vortexing.
15. Quick spin.
16. Add 1200 μL 100% EtOH and mix well by pipetting. Proceed immediately to the next step.
17. Transfer 700 μL of the sample to labeled RNeasy MinElute spin column and centrifuge max speed for 15s.
18. Set aside the flow-through in the collection tube and place column in a new collection tube.
19. Repeat steps 20-21 until the entire sample has passed through the column.
20. Add 500 μL Buffer RPE to the column and centrifuge max speed for 15s.
21. Set aside the flow-through in the collection tube and place column in a new collection tube.
22. Add 500 μL Buffer RPE to the column and centrifuge max speed for 1 min.
23. Set aside the flow-through in the collection tube and place column in a new collection tube.
24. Open the lid of spin column and centrifuge max speed for 5 min.
25. Discard the collection tube and place the column in a new 1.5 mL LowBind microcentrifuge elution tube.
26. Add 30 μL RNase-free water directly to the column membrane and incubate at room temperature for 1 min.
27. Centrifuge max speed for 1 min to elute the RNA.
28. Quantitate RNA using NanoDrop Spectrophotometer, record ng/μL, 260/280, 260/230.
29. Evaluate RNA integrity using Agilent RNA 6000 Nano Kit.

The method disclosed below can be used for completion of tissue lysis and DNA extraction.

### Part 3: Completion of Tissue Lysis and DNA Extraction

1. Add 150 μL of tissue lysis solution to prior remaining lysate not used for RNA extraction, to yield ˜300 μL. (Tissue lysis solution=120 μL ATL+30 μL proteinase K [pro-K]).
2. Verify tube label intact.
3. Place in heated rotisserie set to 65° C. for additional 1-16 hours to ensure that tissue is completely digested.
4. Add 25 μL pro-K and repeat step 3 if unlysed tissue remains.
5. Remove tubes from rotisserie and quickspin.
6. If performing assays requiring RNA-free DNA: Add 1.5 μL RNaseA, vortex for 3-5s, quick spin, and incubate at RT for 5 min.
7. To a purple Qiaquick spin column (PQC), add 490 μL of Binding Buffer PM plus 10 μL of 3M sodium acetate, then add 150 μL aqueous phase lysate and pipette up and down 5 times (thus there will remain ˜150 μL remaining unused lysate for storage, direct bisuLfite conversion, etc.). Note: Do not pipette off the organic phase. Verify tube label intact. Save lysate tube with residual lysate indefinitely.
8. Double Bind: Centrifuge PQC at 2,000 rpm for 90s to collect flow-through. Note: not all of the specimens may go through the filter at this point. Repeat step 7 if needed.
9. The flow-through contains unbound DNA. Re-apply the flow-through to the same PQC and spin again at 2,000 rpm for 90s. Set aside and save the resultant flow-through until recovery of eluted DNA is verified.
10. Add 700 μL of Buffer PE and centrifuge at 1,000 rpm for 15s. Change collection tubes.
11. Add 700 μL of 80% EtOH and centrifuge at max rpm for 60s.
12. Change collection tubes, centrifuge at max speed for 5 mins.
13. Discard collection tubes. Note the ˜1 μL residual EtOH on the side of the PQC columns, which should be pipetted off. Next, uncap PQC and place into 65° C. heat block for 5 minutes incubation to evaporate off residual EtOH. During incubation, label LOW BIND DNA TUBES for DNA elution.
14. Preheat AE in 65° C. heat block/chamber.
15. Add 60 μL of AE elution buffer (preheated to 65° C.) to column filter directly, and incubate for 1 min in heat block at 65° C.
16. Centrifuge at max speed for 1 min.
17. Double Elute: The PQC contains uneluted DNA. Re-apply the first elution to same PQC, then centrifuge again at max speed for 1 min.
18. Verify sample label on tube.
19. Nanodrop eluted DNAs: record A260/280, A260/230, and ng/μL on sample tracking worksheet.
20. If eluted DNA requires further concentration and/or further purification, proceed to Zymo DNA clean and concentrate step. NOTE: FFPE DNA purification/concentration to remove impurities including melanin should be performed with the “Clean and Concentrate” kit.
21. Qubit™ the eluted DNAs: record ng/μL on tracking worksheet.
NOTE: DNA is stable in lysis solution, and the lysis in the heated rotisserie may be extended over the weekend if necessary.

The method provided below can be used for DNA cleaning (optional) and concentrating.

### Optional DNA Clean and Concentrate

1. Add 7 volumes of DNA Binding Buffer to DNA sample.
2. Double Bind: Load the mixture into Zymo-Spin column and centrifuge at max speed for 30s. Re-apply the flow through and centrifuge again at max speed for 30s.
1. Place column in a new collection tube and set aside the flow through.
2. Add 200 μL of Wash Buffer and centrifuge at max speed for 30s.
3. Discard the flow through and repeat the wash step.
4. Discard the flow through and centrifuge for an additional 30s.
5. Place the column into a new collection tube.
6. Add 10 μL of dH₂O (preheated to 65° C.) and incubate for 1 min in thermomixer at 65° C. Note: elution volume can be adjusted, based on expected yield.
7. Centrifuge at max speed for 1 min.
8. Double elute: Re-apply the elution to the column, incubate for 1 min in thermomixer at 65° C., then centrifuge at max speed for 1 min.
9. Re-quantify DNA using Nanodrop.

Provided below is a method for ddPCR-based molecular target quantitation in archival samples.

### B. Protocol: DdPCR-Based Molecular Target Quantitation Method for Archival Samples

Materials, samples and controls used for ddPCR-based molecular target quantitation in archival samples is provided below.

### 1. Samples & Controls

1) GeneCopoeia ORF cDNA clones (No Longer used, transitioned all controls to cell lines (MCF7 & MB-231)
i. Oncotype Estrogen Gene: ESR1 (A0322), PGR (A1694), BCL2 (H3307), SCUBE2 (T8649)
ii. Proliferation Gene: AURKA (Z0617), BIRC5 (A3492), CCNB1 (B0252), MYBL2 (B0073), TK1 (A8372)
iii. Her2 Gene: ERBB2/HER2 (Z2866)
iv. Reference Gene: B2M (10035), CALM2 (Z0597), PUM1 (E0087))
2) TissueScan, Breast Cancer cDNA Array I (OriGene, BCRT101)
i. C11 (ER+PR+HER2+w)
ii. C08 (Triple Negative)
iii. F11 (ER+PG+HER2−)
3) Breast Cancer Cell Lines
i. MDA-MB-231 (ATCC, CRM-HTB-26D), sample T25
ii. MCF-7 (ATCC, HTB-22), sample T24
4) 20-year-old UAMS Breast Cancer samples (13 samples), RNA extracted by “Protocol: Nucleic Acid Extraction from Archival Specimens” the custom protocol disclosed in Section II.
i. T1-T13
5) S8897 extracted RNA (23 samples). RNA extracted by “Protocol: Nucleic Acid Extraction from Archival Specimens” described in Section II.
i. Normal Lymph Node (13 samples): LN1-LN13
ii. Tumor (10 samples): T14-T23

Provided below are reagents and accessories required for the described methods.

### 2. Reagent & Accessories

### Cell Culture

1) DMEM/F12 (Gibco, Cat #10565-108)
2) RPMI 1640 (Gibco, Cat #A10494-01)
3) Heat inactivated Fetal Bovine Serum, HI FBS (Gibco, Cat #10082-147)
4) DPBS, no calcium, no magnesium (Gibco, Cat #14190-144)
5) Penicillin-Streptomycin Solution, 100× (Corning, Cat #30-002-CI)
6) Trypsin EDTA 1×, 0.05% Trypsin 0.53 mM EDTA (Corning, Cat #25-052-CI)

### Nucleic Acid Extraction and Reverse Transcription

7) Protocol: Nucleic Acid Extraction from Archival Specimens, the custom protocol disclosed in Section II.

### Reverse Transcription

8) SuperScript VILO cDNA Synthesis Kit (Invitrogen, #11754-050) ddPCR for preamplification
9) 20× TaqMan PreAmp Master Mix (ThermoFisher Scientific, Cat #4391128) 10) 2× SsoAdvanced PreAmp Supermix (Bio-Rad, Cat #172-5160)
11) PrimePCR PreAmp Assays for target gene (BioRad)
12) 2× ddPCR Supermix for Probes, No dUTP (Bio-Rad, Cat #186-3023)
13) Low TE buffer or Nuclease-free water (TEKNOVA, Cat #T0221)
14) TaqMan Gene expression Assays (20×) for Target and Reference Gene

TABLE 2
ddPCR gene assays

| | | | | | Amplicon | |
|---|---|---|---|---|---|---|
| Classification | Gene | Assay No. | Company | Assay_ID | (bp) | Reporter |
| Estrogen | ESR1 | E1 | BioRad | dHsaCPE5033300 | 77 | FAM |
| Genes | | E2 | ThermoFisher | Hs01046816_m1 | 65 | FAM |
| | | E3 | ThermoFisher | Hs00174860_m1 | 62 | FAM |
| | | E4 | ThermoFisher | Hs01046813_m1 | 66 | FAM |
| | PGR | P1 | BioRad | dHsaCPE5058418 | 120 | FAM |
| | | P2 | ThermoFisher | Hs01556707_m1 | 102 | FAM |
| | | P3 | ThermoFisher | Hs01556702_m1 | 77 | FAM |
| | | P4 | ThermoFisher | Hs01556701_m1 | 84 | FAM |
| | | P5 | ThermoFisher | Hs00612017_s1 | 73 | FAM |
| | | P6 | ThermoFisher | Hs04332988_m1 | 88 | FAM |
| | BCL2 | BC1 | BioRad | dHsaCPE5045926 | 108 | FAM |
| | | BC2 | ThermoFisher | Hs00608023_m1 | 81 | FAM |
| | | BC3 | ThermoFisher | Hs04986394_s1 | 73 | FAM |
| | SCUBE2 | SC1 | BioRad | dHsaCPE5041248 | 103 | FAM |
| | | SC2 | ThermoFisher | Hs00221277_m1 | 64 | FAM |
| | | SC3 | ThermoFisher | Hs01012951_m1 | 73 | FAM |
| | | SC4 | ThermoFisher | Hs01012957_m1 | 73 | FAM |
| HER2 | ERBB2- | H1 | BioRad | dHsaCPE5037554 | 78 | FAM |
| Genes | HER2 | H2 | ThermoFisher | Hs01001598_g1 | 55 | FAM |
| | | H3 | ThermoFisher | Hs01001587_g1 | 57 | FAM |
| | | H4 | ThermoFisher | Hs01001587_g1 | 57 | FAM |
| | GRB7 | G1 | ThermoFisher | Hs00917999_g1 | 68 | FAM |
| | | G2 | ThermoFisher | Hs00918011_g1 | 57 | FAM |
| | | G3 | ThermoFisher | Hs00918005_g1 | 57 | FAM |
| Proliferation | MKi67 | K1 | BioRad | dHsaCPE5050322 | 108 | FAM |
| Genes | | K2 | ThermoFisher | Hs01032437_m1 | 77 | FAM |
| | | K3 | ThermoFisher | Hs01032443_m1 | 66 | FAM |
| | | K4 | ThermoFisher | Hs01032432_g1 | 69 | FAM |
| | | K5 | ThermoFisher | Hs00267195_m1 | 78 | FAM |
| | | K6 | ThermoFisher | Hs04260396_g1 | 64 | FAM |
| | | K7 | ThermoFisher | Hs04981441_m1 | 68 | FAM |
| | AURKA | A1 | BioRad | dHsaCPE5057812 | 138 | FAM |
| | | A2 | ThermoFisher | Hs00269212_m1 | 85 | FAM |
| | | A3 | ThermoFisher | Hs01582072_m1 | 146 | FAM |
| | | A4 | ThermoFisher | Hs01582073_m1 | 117 | FAM |
| | BIRC5 | BR1 | BioRad | dHsaCPE5025654 | 100 | FAM |
| | | BR2 | ThermoFisher | Hs04194392_s1 | 102 | FAM |
| | | BR3 | ThermoFisher | Hs03063352_s1 | 66 | FAM |
| | | BR4 | ThermoFisher | Hs00153353_m1 | 93 | FAM |
| | | BR5 | ThermoFisher | Hs00977612_mH | 79 | FAM |
| | CCNB1 | CC1 | BioRad | dHsaCPE5036646 | 111 | FAM |
| | | CC2 | ThermoFisher | Hs01030099_m1 | 86 | FAM |
| | | CC3 | ThermoFisher | Hs01030098_g1 | 108 | FAM |
| | | CC4 | ThermoFisher | Hs01030097_m1 | 66 | FAM |
| | MYBL2 | MY1 | BioRad | dHsaCPE5053700 | 73 | FAM |
| | | MY2 | ThermoFisher | Hs00942540_m1 | 71 | FAM |
| | | MY3 | ThermoFisher | Hs00942547_m1 | 66 | FAM |
| | | MY4 | ThermoFisher | Hs00942543_m1 | 62 | FAM |
| | TK1 | T1 | BioRad | dHsaCPE5040182 | 67 | FAM |
| | | T2 | ThermoFisher | Hs01062125_m1 | 67 | FAM |
| | | T3 | ThermoFisher | Hs01062126_g1 | 59 | FAM |
| | | T4 | ThermoFisher | Hs01062123_m1 | 78 | FAM |
| Standard | B2M | B1 | BioRad | dHsaCPE5053100 | 123 | FAM |
| Reference | | B2 | ThermoFisher | Hs99999907_m1 | 75 | VIC |
| Genes | | B3 | ThermoFisher | Hs00187842_m1 | 64 | VIC |
| | CALM2 | C1 | BioRad | dHsaCPE5037784 | 60 | FAM |
| | | C2 | ThermoFisher | Hs01572631_m1 | 73 | VIC |
| | | C3 | ThermoFisher | Hs04187148_g1 | 95 | VIC |
| | PUM1 | U1 | BioRad | dHsaCPE5049608 | 123 | FAM |
| | | U2 | ThermoFisher | Hs00472881_m1 | 77 | VIC |

15) ddPCR droplet generation (DG) oil for probes (Bio-Rad, Cat #186-3005)
16) ddPCR Buffer Control for Probes (Bio-Rad, Cat #186-3052)
17) DG8 droplet generator cartridges (Bio-Rad, Cat #186-4008)
18) DG8 gaskets (Bio-Rad, Cat #186-3009)
19) DG8 droplet generator cartridge holder (Bio-Rad, Cat #186-3051)
20) DNA LoBind Tube, 1.5 ml (Eppendorf, Cat #022431021)
21) 96-well plates (Eppendorf, Cat #951020303)
22) PCR Plate Heat Seal, foil, pierceable (Bio-Rad, Cat #181-4040)

The equipment and software required for the methods described are provided below.

### 3. Equipment

1) C02 incubator (ThermoFisher, Cat #51030-403)
2) Centrifuge 5804R (Eppendorf, Cat #022628146)
3) Microcentrifuge (Eppendorf, Cat #022620623)
4) QX200 droplet generator (Bio-Rad, Cat #186-4002)
5) QX200 droplet reader (Bio-Rad, Cat #186-4003)
6) T100 Thermal cycler (Bio-Rad, Cat #186-1096)
7) PX1 PCR Plate Sealer (Bio-Rad, Cat #181-4000)
8) NanoDrop 2000 Spectrophotometers (ThermoFisher Scientific, Cat #ND-2000)
9) Mini-Centrifuge (Fisher Scientific, S67601B)

### Software: QuantaSoft Analysis Pro (Version 1.0.596)

### 4. Procedure

Provided below is the procedure for sample preparation.

### 1. Sample Preparation, Controls & FFPE Samples

1) ORF cDNA clones were purchased from GeneCopoeia.
2) Breast cancer cDNA array was purchased from OriGene.
3) Breast Cancer Cell lines
i. MDA-MB-231 and MCF-7 breast cancer cell lines were obtained from American Type Culture Collection (ATCC) and maintained in DMEM/F12 and RPMI 1640 supplemented with 10% FBS and 1% penicillin/streptomycin respectively.
ii. RNA was extracted using Quick DNA/RNA MiniPrep Plus kit (Zymo Research, D7003) follow the manufacturer's instructions.
a. Collection: MDA-MB-231 and MCF-7 were harvested at the 0.5×106 of cell concentration with PBS in a DNase/RNase free microcentrifuge tube.
b. Lysis and Purification
   a) Make pellet by centrifugation at 200×g for 3 minutes and resuspend the cell pellet in DNA/RNA Lysis buffer.
   b) Transfer the lysed sample into a Spin-Away Filter in a collection tube and centrifuge at 10,000×g for 30 seconds at room temperature which is the standard condition and save the flow-through in the collection tube for RNA purification.
   c) Add the equal volume of 100% ethanol to the flow-though and mix well.
   d) Transfer the samples into a Zymo-Spin IIICG Column in a collection tube and centrifuge at 10,000×g for 30 seconds at room temperature. Discard the flow-through.
c. Washing and Elution
   a) Add 400 μl of DNA/RNA Prep Buffer to the column and centrifuge. Discard the flow-through.
   b) Add 700 μl of DNA/RNA Wash Buffer to the column and centrifuge. Discard the flow-through.
   c) Add 400 μl of DNA/RNA Wash Buffer to the column and centrifuge the column at 10,000×g for 2 minutes to remove the residual buffer on the column. Transfer the column into a fresh microcentrifuge tube carefully.
   d) Add 50 μl of DNase/RNase-Free water on to the matrix of column at the center and let stand for 1 minute, then centrifuge to obtain RNA in the microcentrifuge tube. Keep the RNA at −80° C. until use.
d. The purity and concentration of extracted RNA were measured by NanoDrop 2000.
iii. cDNA was synthesized using SuperScript VILO cDNA Synthesis Kit (Invitrogen, #11754-050) follow the manufacturer's instructions.
a. One microgram of RNA sample was subjected to reverse transcription as shown in Table 3 and components were mixed well on ice.

TABLE 3
Components for reverse transcription

| Component | Volume | |
|---|---|---|
| 5X VILO Reaction Mix | 4 | μl |
| 10X SuperScript Enzyme Mix | 2 | μl |
| RNA (1.0 μg) | x | μl |
| DEPC-treated water | to 20 | μl |

b. Samples were placed in thermocycler and was run as per the following protocol: 25° C. for 10 minutes, 42° C. for 60 minutes, 85° C. at 5 minutes.
c. The purity and concentration of synthesized cDNA were measured by NanoDrop 2000. It was considered as ‘an ideal state of DNA’ between 1.7 and 1.8 and ‘a pure state of DNA’ between 1.8 and 2.0 at the ratio of A260/280. Keep the cDNA at ˜80° C. until use.
4) UAMS Breast Cancer FFPE Blocks (20 yrs old) and, SWOG S8897 Breast Cancer & Normal LN Slides (˜30 yrs old)
i. The 20 year old UAMS breast cancer FFPE blocks and, the S8897 breast cancer slides and matched normal lymph node sample slides (˜30 years old) underwent nucleic acid extraction via the custom protocol, “Protocol: Nucleic Acid Extraction from Archival Specimens” described in Section II. Note: The 20 year old UAMS Breast Cancer FFPE blocks were sectioned at the 5-6 μm thickness and placed onto glass slides for nucleic acid extraction. The S8897 FFPE samples were pre-cut and mounted on glass slides −30 years ago.
ii. cDNA was synthesized using SuperScript VILO cDNA Synthesis Kit (Invitrogen, #11754-050).
   2. Preparation of Preamplification Reaction cDNA Product

Provided below are the methods for preparing preamplification reactions using SsoAdvanced (method 1) and TaqMan (method 2).

### Method 1: SsoAdvanced PreAmp Supermix (Bio-Rad)

1) Thaw SsoAdvanced PreAmp Supermix at room temperature. Mix thoroughly, then centrifuge briefly to collect the solution at the bottom of the tube. Store on ice.

2) In order to make Preamplification assay pool, add 5 μl of PrimePCR PreAmp assay for each target gene (final concentration is 0.01×) and Nuclear-free water to get a total volume of 50 μl in a microcentrifuge tube and mix thoroughly (Table 4), and then centrifuge briefly. Prepare preamplification reaction mix on ice according to the following instructions.

3) Good pipetting practice must be employed to ensure assay precision and accuracy.

TABLE 4
Preamplification components

| | Volume in a 50 | | Final |
|---|---|---|---|
| Component | μl Reaction | | Concentration |
| SsoAdvanced PreAmp Supermix (2x) | 25.0 | μl | 1x |
| Preamplification assay pool | 5.0 | μl | 0.01x |
| (0.01x TaqMan) | | | |
| cDNA template | Variable | | |
| Nuclease-free water | Variable | | |
| Total preamplification reaction | 50.0 | μl | — |
| mix volume | | | |

4) Mix the reaction mix thoroughly to ensure homogeneity and samples were placed in thermocycler and was run as the shown in Table 5.

TABLE 5
Thermocycler setting

| Thermal | Polymerase | | Annealing/ | |
|---|---|---|---|---|
| Cycler | Activation | Denaturation | Extension | Hold |
| Bio-Rad | 95° C., 3 | 95° C., 15 | 58° C., 4 | 4° C., |
| T100 | min. 1x | sec x10 | min x10 | Infinite |

5) After run completion, the pre-amplified cDNA product should be diluted 1:5 using TE buffer. The diluted pre-amplified cDNA product can be stored at −20° C. for up to 12 months or 4° C. for up to 72 hr.

6) Prepare the components of the reaction in a 8-strip PCR tube for preamplification ddPCR with preamplification reaction mix above and ddPCR reaction with original cDNA as shown in Table 6.

TABLE 6
Preamplification ddPCR with preamplification
reaction mix and ddPCR reaction

| | Preamplification | | | | Final |
|---|---|---|---|---|---|
| Content | ddPCR | | ddPCR | | Concentration |
| 2× ddPCR Supermix | 10 | μl | 10 | μl | 1x |
| 20× target or reference | 1 | μl | 1 | μl | 1x |
| primer/TaqMan probe | | | | | |
| (FAM or VIC or HEX) | | | | | |
| Preamplified cDNA | 1 | μl | x | | 1/250 of |
| products (diluted 1:5) | | | | | original cDNA |
| Original cDNA | X | | 1 | μl | |
| Nuclease free water | 4 | μl | 8 | μl | |
| Total volume | 20 | μl | 20 | μl | |

7) After attaching caps on the 8-strip PCR tube, slightly vortex the PCR tube and briefly centrifuge the PCR tube to ensure the contents of the reaction are at the bottom of the well.

### Method 2: TaqMan PreAmp Supermix (ThermoFisher)

1) Thaw TaqMan PreAmp Master Mix at room temperature. Mix thoroughly, then centrifuge briefly to collect the solution at the bottom of the tube. Store on ice.

2) To make TaqMan assay Pool, combine equal volume of the 12 target gene and 3 standard reference of TaqMan gene expression assays in a microcentrifuge tube and dilute the pooled assays using 1× TE buffer to be each assay at a final concentration of 0.2×. Store at 4° C. up to 30 days at −20° C. up to 1 year.

3) Prepare preamplification reaction mix on ice according to Table 7. Good pipetting practice must be employed to ensure assay precision and accuracy.

TABLE 7
Preamplification reaction mix preparation

| | Volume in a | | Final |
|---|---|---|---|
| Component | 50 μl Reaction | | Concentration |
| TaqMan PreAmp Supermix (2x) | 25.0 | μl | 1x |
| TaqMan assay pool (0.2x TaqMan) | 12.5 | μl | 0.2x |
| cDNA template | 5.0 | μl | 1/10 of original |
| | | | cDNA |
| Nuclease-free water | 7.5 | μl | |
| Total preamplification | 50.0 | μl | — |
| reaction mix volume | | | |

4) Mix the reaction mix thoroughly to ensure homogeneity and samples were placed in thermocycler and was run as shown in Table 8.

TABLE 8
Thermocycler setting

| Thermal | Enzyme | | Annealing/ | Enzyme | |
|---|---|---|---|---|---|
| Cycler | Activation | Denaturation | Extension | inactivation | Hold |
| Bio-Rad | 95° C., 10 | 95° C., 15 | 60° C., | 99° C., 10 | 4° C., |
| T100 | min, x1 | sec, x10 | 4 min, x10 | min, x1 | Infinite |

After run completion, the preamplfied cDNA product can be stored at −20° C. up to 7 days.

5) Prepare the components of the reaction in a 8-strip PCR tube for preamplification ddPCR with preamplification reaction mix above and ddPCR reaction with original cDNA as shown in Table 9:

TABLE 9
Preamplification ddPCR and ddPCR reaction mix

| | Preamplification | | | | Final |
|---|---|---|---|---|---|
| Content | ddPCR | | ddPCR | | Concentration |
| 2× ddPCR Supermix | 10 | μl | 10 | μl | 1x |
| 20× target or reference | 1 | μl | 1 | μl | 1x |
| primer/TaqMan probe | | | | | |
| mix (FAM or VIC or HEX) | | | | | |
| Preamplified cDNA | 5 | μl | x | | 1/10 of |
| products (diluted 1:5) | | | | | original cDNA |
| Original cDNA | X | | 1 | μl | |
| Nuclease free water | 4 | μl | 8 | μl | |
| Total volume | 20 | μl | 20 | μl | |

6) After attaching caps on the 8-strip PCR tube, slightly vortex the PCR tube and briefly centrifuge the PCR tube to ensure the contents of the reaction are at the bottom of the well.

3. ddPCR Reaction: Droplet Generation and PCR

Provided below are steps performing droplet generation and ddPCR reaction.

7) After assemble DG8 droplet generator cartridges into cartridge holder, transfer 20 μl of the ddPCR reaction mix or preamplified ddPCR mix into the center well of the cartridge which designated sample. Avoid pipetting any air bubbles into the well as this may prevent droplet generation.

8) Dispense 70 μl droplet generator oil into the wells of the cartridge designated “oil (Bottom)”.

9) Attach DG8 gaskets to the top of the holder/cartridge and insert the holder/cartridge into the QX200 Droplet Generator to produce millions of sample droplets. When droplets are generated successfully, the wells will appear slightly opaque.

10) Transfer the 40 μl of the droplet from the cartridge holder into a 96-well plate PCR plate.

11) After seal the plate with an easy pierce thermal foil for PCR plate, run the ddPCR in a thermal cycler using the manufacturer's standard protocol, as shown in Table 10.

TABLE 10
Thermal cycler settings for ddPCR

| | | | number of |
|---|---|---|---|
| Cycling step | Temperature | Time | cycles |

| | | | | | |
|---|---|---|---|---|---|
| Enzyme activation | 95° | C. | 10 | min | 1 |
| Denaturation | 94° | C. | 30 | sec | 40 |
| Anneal/extend | 60° | C. | 60 | sec | |
| Enzyme deactivation | 98° | C. | 10 | min | 1 |

| | | | | |
|---|---|---|---|---|
| Hold | 4° | C. | Infinite | 1 |

### 4. Droplet Reading and Analysis

The method involved in droplet reading and analysis is provided below.

1) Secure the PCR plate containing the droplets in the plate reader holder.

2) Enter experiment information into the template and apply the following to each well:
a. Sample: Name, Experiment-ABS (Absolute Quantification), Supermix-ddPCR Supermix for Probes (no UTP)
b. Target 1: Name, Ch1 Unknown (FAM)
c. Target 2: Name, Ch2 Unknown (VIC)

3) After putting the experiment information, start the plate run.

4) Select the dye pair in use (FAM/VIC) and the direction for the wells to be read (by column or by row), then QX200 begin to read the information of droplet.

5) After save the ddPCR file, open the QuantaSoft Analysis Pro to analyze the data.

6) Open the ddPCR file on the QuantaSoft Analysis Pro Software (BioRad, version 1.0), set the threshold to separate the positive and negative droplets each well.

7) Export the analyzed data to Excel file.

### 5. Molecular Probe Selection for Gene Targets

A metaheuristic strategy akin to natural selection was employed for the selection of gene target probes. This approach was coupled with extensive testing on challenging samples in order to optimize the choice of probes for each gene target. A minimax optimization strategy was utilized for the generational survival of probes to their gene targets. The selection procedure sought to minimize the amplicon size of the chosen probe along with maximizing the performance of the probe's sensitivity and specificity on a series of reference materials. Reference materials consisted of cell lines and clinical samples having a known molecular phenotype.

C. Initial Processing and Normalization of ddPCR Data

Raw droplet digital polymerase chain reaction (ddPCR) data consisted of multiple gene targets and each gene target consisted of multiple probes. In essence the measured values by the individual probes correspond to the mRNA gene expression quantity detected in the sample. As a pre-processing step, on a sample-by-sample basis only the maximum value among the probes for a given gene target was selected for subsequent processing. Next, each sample dataset was individually (self) normalized using a z-score like method, specifically by following:

$${x}^{′} = ( x - \overline{x} ) / SD$$

where x is the observed value, x is the mean and SD is the standard deviation of the molecular targets within that particular ddPCR sample.
D. Statistical Simulation of ddPCR Data

The experimental data was resampled by means of a non-parametric bootstrap approach in order to derive more robust estimates via distance geometry plots of the actual experimental samples (breast cancer subtypes, lymph nodes). A set of simulated (synthetic) datasets were created and used to approximate aspects of sampling distributions. This provides an ability to explore to some degree, the expected variation if, the ddPCR experiments were repeated. The bootstrap is an efficient way to obtain an estimate of the experimental data sampling distribution without the need for any distributional assumptions or, constructing a generative model for the creation of new datasets. The bootstrap procedure was performed with replacement meaning the same datapoint may appear multiple times in a resampled dataset.

A multilevel structure approach was employed for the non-parametric bootstrap resampling procedure. The datasets from each breast cancer subtype (luminal A, luminal B, Her2 enriched, basal type) and lymph nodes were segregated, along with the corresponding probes used for each of the molecular targets. Each breast cancer subtype was resampled separately. Lymph nodes were also resampled separately as a single group. Following the resampling procedure, each simulated sample dataset (breast cancer subtype, lymph node) contained values for all of the probes, which correspond for all of the molecular targets. The simulated (synthetic) sample datasets were then processed and subject to further analyses in an identical manner as the actual sample data sets.

E. Distance Geometry and Visualization Approaches Applied to ddPCR Data

Distance geometry type analyses of all data (actual and synthetic) were performed using the R Statistical Computing and Graphics framework, version 4.1.2 and RStudio version 2022.02.0. Hierarchical clustering analysis (HCA) plots were aggregated and performed on a sample-by-sample basis. Additionally, HCA plots were also constructed and aggregated on a sample versus molecular targets basis. In all instances, the corresponding heatmaps were generated using the heatmap.2 function from the gplots v3.1.3 library, using default (“complete”) clustering and the Euclidean distance measure. Principal component analysis (PCA) values were calculated using the prcomp function from the stats library (part of the R core package), and visualization was displayed using the ggplot function from the ggplot2 v3.3.6 library. Finally, a visualization utilizing dot plots displaying the molecular targets (x-axis) versus z-score transformed data (y-axis) for each of the ddPCR samples was displayed using the ggplot function from the ggplot2 library.

### F. RNA-Seq Sample Prep and Bioinformatics

### RNA-Seq Sample Preparation

RNA-seq sample preparation utilized the SMARTer Stranded Total RNA-seq kit v2—Pico (TaKaRa, cat #634411) due to the low input requirements and the ability to accommodate intact or degraded sample material. An Illumina HiSeq 3000 with 75 bp PE was utilized for all RNA NGS studies.

### Bioinformatic Pipeline Processing

RNA-seq samples were first demultiplexed and FastQ files were created from BCL files using bcl2fastq2 (adapter trimming was additionally performed during the conversion). FastQC was used to assess the quality of FastQ files. STAR was used to align each sample's paired-end reads to the Ensembl Human reference genome build GRCm38 (using STAR's “2-pass” method). Quality control and assessment of resulting BAM files was performed using QualiMap and RNA-SeQC. Picard was used to add read group information. The marking of duplicate reads and sorting of aligned files was also done using Sambamba. Each sample's BAM file was initially processed using StringTie, using Ensembl gene annotations to guide transcriptome (limiting output to only annotated genes). The StringTie option to output “Ballgown-ready” files was enabled.

### G. DNA Sequencing Sample Prep and Bioinformatics

### Genomics Sample Preparation and Breast Cancer Panel Processing

The QIAGEN QIAseq Human Breast Cancer Panel (DHS-001Z, 93 Genes) library prep kit was used for targeted DNA-based assays involving tumor and normal (T/N). An Illumina HiSeq 3000 with 150 bp PE was utilized for DNA NGS studies. The breast cancer panel, which utilizes uniform molecular identifiers (UMIs) was run with a coverage of ˜2000× for the tumor and 600× for the germline. The Qiagen web portal was utilized for smCounter2-based bioinformatic analyses. The aforementioned pipeline generates aligned reads in BAM format and variants detected in VCF format. Quality control and assessment of resulting BAM files was performed using QualiMap.

### Whole Genome Sample Prep, Alignment and Copy Number Variation Analysis

Whole genome sequencing (WGS) libraries were constructed using the New England BioLabs (NEB) NEBNext Ultra II DNA library prep kit (NEB #E7645, E7103) and sequenced initially in a low-pass manner (˜7-15×) or an ultra-low-pass fashion (˜0.3×). The ultra-low-pass approach was utilized later in the study following the establishment of the ichorCNA v0.2.0 analysis method for copy number analysis (CNA) at ˜0.3× coverage for T/N. Regardless of the depth of sequencing, all specimens were analyzed using ichorCNA.

Following NGS, DNA samples were first demultiplexed and FastQ files were created from BCL files using bcl2fastq2 and adapter trimming was additionally performed during the conversion. FastQC was used to assess the quality of FastQ files. Each sample's FastQ paired-end files were aligned to the Ensembl Homo Sapiens reference genome (build GRCh37.75) using BWA v0.7.12. Quality control and assessment of BAM files was performed with QualiMap. BAM files were post-processed to mark duplicates and sort aligned reads via Sambamba. Copy number data was computationally inferred using the R library ichorCNA v0.2.0.

### Extraction Analysis

For all study specimens, an extraction analysis of gDNA and RNA by: i) Nanodrop, ii) Qubit, iii) Fragment Analysis and iv) a functional assay (Qiagen-QIAseq DNA QuantiMIZE or Roche-KAPPA NGS FFPE DNA QC) to assess FFPE DNA quality by amplification metrics, for all study specimens was performed.

Regarding Nanodrop findings, nucleic acids & proteins have absorbance maxima at 260 and 280 nm and the ratio of absorbances at these wavelengths has been used as a measure of purity. A ratio of ˜1.8 is generally accepted as pure for DNA and ˜2.0 for RNA. Absorbance at 230 nm is accepted as “other contamination”. Although purity ratios and spectral profiles are important indicators of specimen purity, the best indicator of DNA or RNA quality is functionality in a downstream application, e.g., the ability of the material to amplify, which further indicates the likely successful construction of an NGS library, for a sequencing study.

Concerning functional assay testing, for specimens T1-T13 (UAMS, 20-year-old from FFPE blocks), a QuantiMIZE QC Call of High was reported for 6/13 specimens and Low for 7/13. For the S8897 specimens (T14-T33), High Quality was reported for 8 specimens, Low Quality for 3, and 7 between High and Low. Regarding fragmentation analysis, RNA (total RNA) shows extensive fragmentation, with the vast majority in the size range of 10-40 nt. gDNA does not show extensive fragmentation and in general, the yields per case were found to be of an adequate abundance for NGS library construction for both tumor and LN.

### Example 1: Molecular Profiling of Archival Tissue Specimens

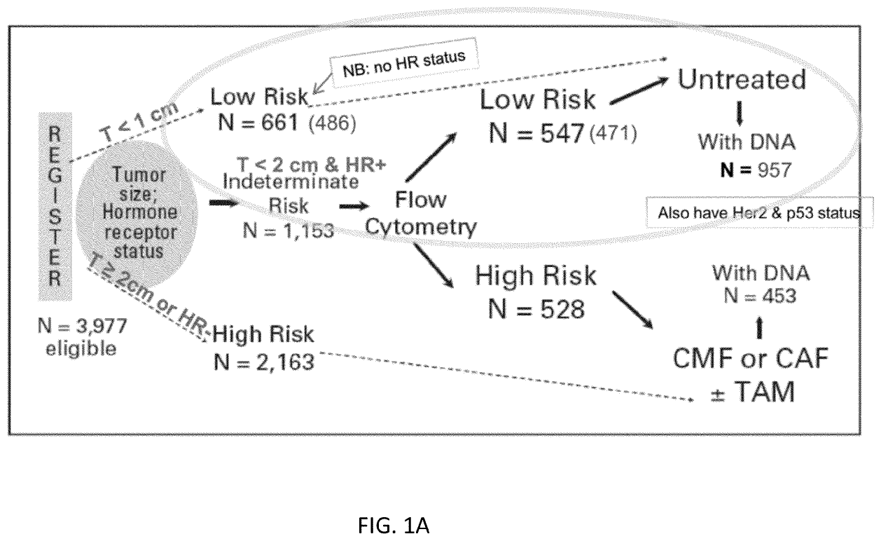

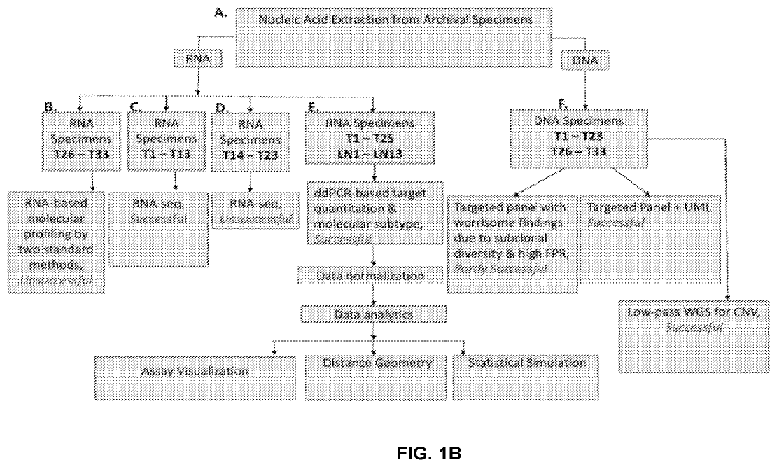

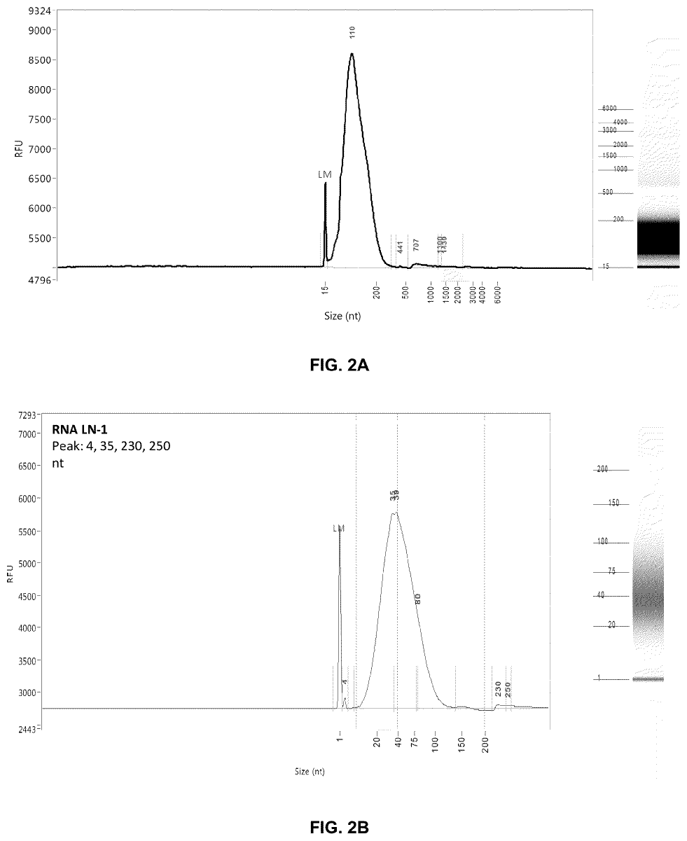

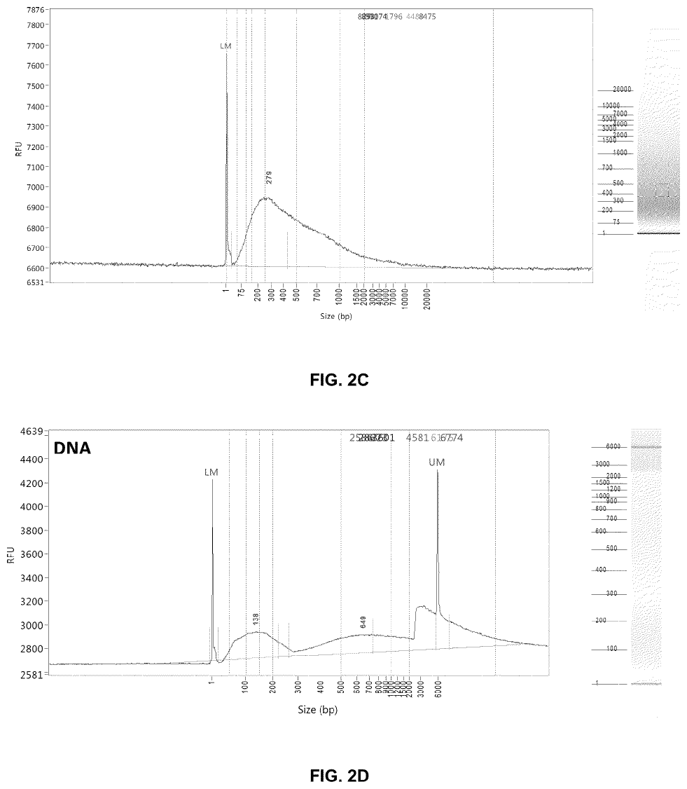

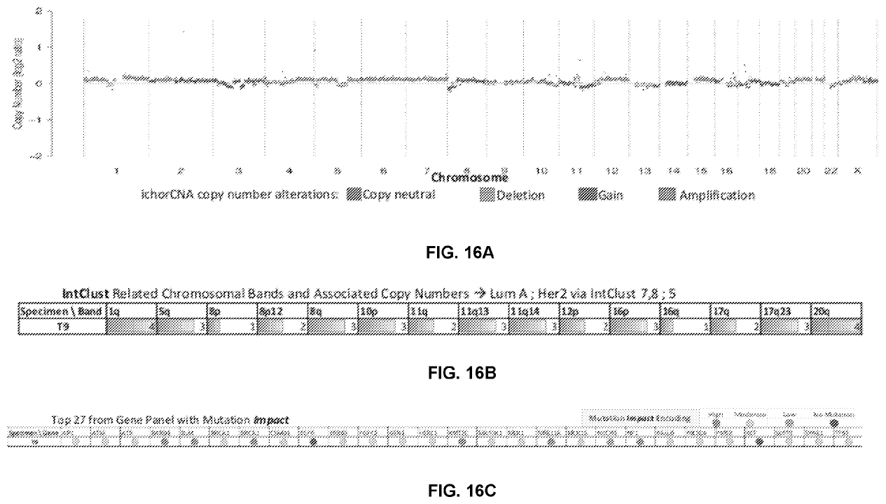

FIG. 1A depicts the S8897 specimen archive used in the study. The S8897 specimen archive comprises specimens from the largest number of patients not treated following surgery for small breast tumors. It is estimated that many patient specimens involve ˜4-6 glass slides of unstained formalin fixed paraffin embedded (FFPE) material that are over ˜20-30 years old. The low risk group criteria were: Patients with T<1 cm that did not have HR status or S-phase flow cytometry evaluated (aka Initial Low Risk Group). Specimens involve glass slides of unstained FFPE material. A flow diagram outlining the archival tissue specimens and their molecular profiling history is shown in FIG. 1B. This figure outlines the overarching phases of this study, whether or not success was achieved, and indicates salient figures, tables, and supplemental materials germane to each study segment. All nucleic acid extractions (“A” of FIG. e B) from archival specimens were performed for both DNA and RNA per the disclosed custom protocol. (Tables 11-22, and FIG. 2A-2n). Several RNA molecular profiling approaches were attempted (“B-E” of FIG. 16) due to the challenging degree of fragmentation. The first attempt (“B” of FIG. 1B) involved specimens from the S8897 cohort (ie, 30-year-old specimens cut on glass slides). The eight specimens with the most RNA were subjected to molecular profiling by two standard methods, namely, i.) Illumina RNA Access and, ii.) a custom NanoString nCounter assay, however both approaches were unsuccessful. Following this unsuccessful external attempt, the inventors conducted all of the molecular profiling.

TABLE 11
Tumor gDNA Metrics and Yields UAMS 20 year
old Breast Cancer FFPE Tissue Blocks

| | | | | | | gDNA |
|---|---|---|---|---|---|---|
| Initial | Final | 260/230 | | | | yield |
| Speci- | Speci- | (2.00- | 260/280 | Nanodrop, | Qubit, | by Qubit, |
| men ID | men ID | 2.20) | (~1.80) | ng/μl | ng/μl | ng |
| S98-3 | T1 | 1.75 | 1.91 | 91.1 | 33.0 | 1320.0 |
| S98-4 | T2 | 2.02 | 1.93 | 51.0 | 31.0 | 1240.0 |
| S98-5 | T3 | 1.94 | 1.90 | 114.3 | 31.1 | 1835.0 |
| S98-6 | T4 | 1.73 | 1.88 | 42.5 | 13.3 | 851.0 |
| S98-8 | T5 | 0.86 | 1.78 | 24.0 | 5.0 | 185.0 |
| S98-9 | T6 | 1.75 | 2.22 | 7.4 | 8.0 | 280.0 |
| S98-11 | 17 | 1.35 | 1.87 | 33.6 | 29.0 | 1015.0 |
| S98-12 | T8 | 1.62 | 1.98 | 12.7 | 11.0 | 385.0 |
| S99-7 | T9 | 2.18 | 1.85 | 398.6 | 125.0 | 3750.0 |
| S99-16 | T10 | 2.15 | 1.92 | 40.2 | 30.0 | 1170.0 |
| S99-17 | T11 | 2.07 | 1.91 | 75.9 | 47.0 | 1880.0 |
| S99-18 | T12 | 1.36 | 2.03 | 12.7 | 9.8 | 539.0 |
| S99-19 | T13 | 1.60 | 1.91 | 21.6 | 20.0 | 1660.0 |

TABLE 12
Tumor gDNA Metrics and Yields, S8897

| Initial | Final | 260/230 | | | | |
|---|---|---|---|---|---|---|
| Speci- | Speci- | (2.00- | 260/280 | Nanodrop | Qubit | gDNA |
| men ID | men ID | 2.20) | (~1.80) | ng/uL | ng/uL | ng |
| S8897DNA2 | T14 | 1.97 | 1.99 | 35.5 | 8.4 | 586.6 |
| S8897DNA3 | T15 | 2.07 | 2.06 | 38.5 | 9.6 | 480.0 |
| S8897DNA11 | T16 | 1.51 | 1.82 | 29.4 | 4.0 | 198.0 |
| S8897DNA12 | T17 | 1.33 | 1.91 | 6.8 | 0.5 | 20.6 |
| S8897DNA13 | T18 | 2.22 | 2.05 | 72.0 | 20.8 | 1040.0 |
| S8897DNA15 | T19 | 2.06 | 2.08 | 58.5 | 14.1 | 1198.5 |
| S8897DNA16 | T20 | 1.66 | 2.14 | 40.8 | 6.5 | 469.4 |
| S8897DNA18 | T21 | 2.08 | 2.04 | 173.0 | 44.0 | 2200.0 |
| S8897DNA19 | T22 | 1.77 | 1.99 | 33.6 | 44.6 | 2230.0 |
| S8897DNA20 | T23 | 1.66 | 2.04 | 5.6 | 0.3 | 13.5 |
| S8897DNA1 | T26 | 2.08 | 1.98 | 96.0 | 24.8 | 2232.0 |
| S8897DNA4 | T27 | 1.78 | 1.84 | 33.0 | 3.3 | 163.0 |
| S8897DNA5 | T28 | 1.91 | 1.91 | 34.5 | 7.7 | 387.0 |
| S8897DNA6 | T29 | 2.20 | 1.87 | 88.0 | 26.2 | 1310.0 |
| S8897DNA7 | T30 | 1.99 | 1.91 | 59.0 | 11.7 | 585.0 |
| S8897DNA8 | T31 | 2.24 | 1.95 | 172.0 | 43.4 | 2170.0 |
| S8897DNA10 | T32 | 2.06 | 1.86 | 66.0 | 8.6 | 567.6 |
| S8897DNA14 | T33 | 2.00 | 2.16 | 38.8 | 6.2 | 466.5 |
| S8897DNA9 | * | 1.41 | 1.77 | 26.8 | 2.8 | 155.1 |
| S8897DNA17 | ** | 1.71 | 1.88 | 16.9 | 6.2 | 338.8 |

* Tissue depleted.
** Not a tumor

TABLE 13
Lymph Node gDNA Metrics and Yields, S8897

| Initial | Final | 260/230 | 260/280 | Nanodrop, | Qubit, | gDNA, |
|---|---|---|---|---|---|---|
| Specimen ID | Specimen | (2.00-2.20) | (~1.80) | ng/μl | ng/μl | ng |
| S8897DNA1LN | LN1 | 2.24 | 1.90 | 478.5 | 26.0 | 1300.0 |
| S8897DNA2LN | LN2 | 2.22 | 1.89 | 632.0 | 27.4 | 1370.0 |
| S8897DNA3LN | LN3 | 1.97 | 1.93 | 45.8 | 7.1 | 640.8 |
| S8897DNA4LN | LN4 | 1.67 | 1.86 | 35.4 | 4.2 | 379.8 |
| S8897DNA5LN | LN5 | 2.12 | 1.94 | 155.5 | 20.6 | 1854.0 |
| S8897DNA6LN | LN6 | 1.86 | 1.90 | 99.5 | 13.5 | 1215.0 |
| S8897DNA11LN | LN7 | 2.11 | 1.92 | 144.0 | 23.8 | 1190.0 |
| S8897DNA13LN | LN8 | 2.06 | 1.96 | 212.0 | 38.8 | 1940.0 |
| S8897DNA14LN | LN9 | 2.15 | 1.90 | 586.5 | 22.2 | 1110.0 |
| S8897DNA16LN | LN10 | 2.25 | 1.90 | 617.5 | 32.2 | 1610.0 |
| S8897DNA17LN | LN11 | 2.17 | 1.93 | 270.5 | 14.4 | 720.0 |
| S8897DNA18LN | LN12 | 2.26 | 2.03 | 204.0 | 6.9 | 346.0 |
| S8897DNA19LN | LN13 | 2.10 | 2.04 | 89.5 | 18.5 | 925.0 |
| S8897DNA7LN | * | 1.78 | 1.91 | 68.4 | 8.4 | 751.5 |
| S8897DNA8LN | * | 1.89 | 1.92 | 121.8 | 13.5 | 1215.0 |
| S8897DNA9LN | * | 2.24 | 1.90 | 663.0 | 20.2 | 1010.0 |
| S8897DNA10LN | ** | | | | | |
| S8897DNA12LN | * | 2.29 | 2.08 | 80.0 | 9.6 | 480.0 |
| S8897DNA15LN | * | 2.17 | 1.91 | 487.0 | 17.8 | 890.0 |
| S8897DNA20LN | * | 2.26 | 2.01 | 125.5 | 19.7 | 985.0 |

* Tissue depleted.
** No LN tissue

TABLE 14
Tumor gDNA Fragment Analysis and Quality Assessment

| Initial | Final | DNA fragment Size Analysis | | | | | | | QuantiMIZE Assay QC | |
|---|---|---|---|---|---|---|---|---|---|---|
| Specimen | Specimen | ≥2000 | ≥1000 | ≥500 | ≥200 | ≥150 | ≥100 | ≥50 | QC | QuantiMIZE |
| ID | ID | bp | bp | bp | bp | bp | bp | bp | Score | QC Call |
| S98-3 | T1 | 0.05 | 0.11 | 0.36 | 0.82 | 0.90 | 0.96 | 0.99 | 0.028 | High |
| DNA | | | | | | | | | | |
| S98-4 | T2 | 0.09 | 0.16 | 0.40 | 0.80 | 0.89 | 0.95 | 0.99 | 0.031 | High |
| DNA | | | | | | | | | | |
| S98-5 | T3 | 0.11 | 0.18 | 0.42 | 0.81 | 0.88 | 0.94 | 0.98 | 0.022 | High |
| DNA | | | | | | | | | | |
| S98-6 | T4 | 0.10 | 0.17 | 0.39 | 0.78 | 0.86 | 0.92 | 0.98 | 0.034 | High |
| DNA | | | | | | | | | | |
| S98-8 | T5 | 0.15 | 0.23 | 0.45 | 0.78 | 0.85 | 0.90 | 0.94 | 0.047 | Low |
| DNA | | | | | | | | | | |
| S98-9 | T6 | 0.10 | 0.15 | 0.51 | 0.78 | 0.85 | 0.91 | 0.95 | 0.042 | Low |
| DNA | | | | | | | | | | |
| S98-11 | T7 | 0.01 | 0.05 | 0.30 | 0.76 | 0.86 | 0.94 | 1.00 | 0.065 | Low |
| DNA | | | | | | | | | | |
| S98-12 | T8 | 0.01 | 0.02 | 0.11 | 0.52 | 0.69 | 0.84 | 0.97 | 0.032 | High |
| DNA | | | | | | | | | | |
| S99-7 | T9 | 0.02 | 0.07 | 0.30 | 0.74 | 0.85 | 0.93 | 0.98 | 0.029 | High |
| DNA | | | | | | | | | | |
| S99-16 | T10 | 0.21 | 0.30 | 0.50 | 0.74 | 0.78 | 0.82 | 0.85 | 0.050 | Low |
| DNA | | | | | | | | | | |
| S99-17 | T11 | 0.15 | 0.25 | 0.47 | 0.77 | 0.86 | 0.92 | 0.96 | 0.056 | Low |
| DNA | | | | | | | | | | |
| S99-18 | T12 | 0.06 | 0.12 | 0.34 | 0.72 | 0.83 | 0.91 | 0.96 | 0.061 | Low |
| DNA | | | | | | | | | | |
| S99-19 | T13 | 0.01 | 0.02 | 0.09 | 0.66 | 0.80 | 0.92 | 0.99 | 0.052 | Low |
| DNA | | | | | | | | | | |

TABLE 15
Tumor gDNA Fragmentation Analysis and Quality Assessment from S8897 Specimens

| | | | | | | | | | KAPA Hu |
|---|---|---|---|---|---|---|---|---|---|
| | | | | | | | | | gDNA QC |
| | | | | | | | | | assay |
| | | | | | | | | | Q129/Q41 |
| | | | | | | | | | High Quality |
| Initial | Final | DNA Fragment Size Analysis | | | | | | | FFPE >0.4, |
| Specimen | Specimen | ≥2000 | ≥1000 | ≥500 | ≥200 | ≥150 | ≥100 | ≥50 | Low Quality |
| ID | ID | bp | bp | bp | bp | bp | bp | bp | FFPE <0.2 |
| S8897DNA2 | T14 | 0.26 | 0.34 | 0.48 | 0.83 | 0.92 | 1.00 | 1.00 | 0.49 |
| S8897DNA3 | T15 | 0.30 | 0.39 | 0.53 | 0.85 | 0.94 | 1.00 | 1.00 | 0.74 |
| S8897DNA11 | T16 | 0.10 | 0.14 | 0.25 | 0.58 | 0.69 | 0.83 | 0.99 | 0.39 |
| S8897DNA12 | T17 | 0.14 | 0.18 | 0.30 | 0.70 | 0.80 | 0.92 | 1.00 | 0.33 |
| S8897DNA13 | T18 | 0.27 | 0.38 | 0.60 | 0.90 | 0.97 | 1.00 | 1.00 | 0.61 |
| S8897DNA15 | T19 | 0.13 | 0.18 | 0.37 | 0.76 | 0.85 | 0.94 | 1.00 | 0.21 |
| S8897DNA16 | T20 | 0.09 | 0.12 | 0.25 | 0.63 | 0.74 | 0.85 | 1.00 | 0.16 |
| S8897DNA18 | T21 | 0.32 | 0.44 | 0.70 | 0.97 | 1.03 | 1.00 | 1.00 | 0.40 |
| S8897DNA19 | T22 | 0.20 | 0.29 | 0.47 | 0.83 | 0.91 | 0.99 | 1.00 | 0.50 |
| S8897DNA20 | T23 | 0.11 | 0.14 | 0.22 | 0.62 | 0.72 | 0.83 | 1.00 | 0.09 |
| S8897DNA1 | T26 | 0.30 | 0.40 | 0.57 | 0.89 | 0.97 | 1.00 | 1.00 | 0.90 |
| S8897DNA4 | T27 | 0.07 | 0.08 | 0.17 | 0.39 | 0.49 | 0.65 | 0.95 | 0.14 |
| S8897DNA5 | T28 | 0.23 | 0.32 | 0.48 | 0.81 | 0.90 | 0.98 | 1.00 | 0.45 |
| S8897DNA6 | T29 | 0.40 | 0.49 | 0.60 | 0.92 | 1.00 | 1.00 | 1.00 | 0.57 |
| S8897DNA7 | T30 | 0.16 | 0.22 | 0.38 | 0.71 | 0.81 | 0.92 | 1.00 | 0.32 |
| S8897DNA8 | T31 | 0.20 | 0.27 | 0.43 | 0.78 | 0.88 | 0.98 | 1.00 | 0.46 |
| S8897DNA10 | T32 | 0.08 | 0.12 | 0.25 | 0.59 | 0.71 | 0.85 | 1.00 | 0.38 |
| S8897DNA14 | T33 | 0.06 | 0.09 | 0.18 | 0.53 | 0.65 | 0.78 | 0.97 | 0.33 |
| S8897DNA9 | * | 0.07 | 0.09 | 0.16 | 0.45 | 0.56 | 0.71 | 0.95 | 0.21 |
| S8897DNA17 | ** | 0.24 | 0.33 | 0.52 | 0.79 | 0.87 | 0.94 | 1.00 | 0.32 |

* Tissue depleted.
** Not a tumor

TABLE 16
LN gDNA Fragmentation Analysis and Quality Assessment from S8897 Specimens

| | | | | | | | | | KAPA Hu |
|---|---|---|---|---|---|---|---|---|---|
| | | | | | | | | | gDNA QC |
| | | | | | | | | | assay |
| | | | | | | | | | Q129/Q41 |
| | | | | | | | | | High Quality |
| Initial | Final | DNA fragment Size Analysis | | | | | | | FFPE >0.4, |
| Specimen | Specimen | ≥2000 | ≥1000 | ≥500 | ≥200 | ≥150 | ≥100 | ≥50 | Low Quality |
| ID | ID | bp | bp | bp | bp | bp | bp | bp | FFPE <0.2 |
| S8897DNA1LN | LN1 | 0.43 | 0.48 | 0.68 | 0.88 | 0.99 | 1 | 1 | 0.56 |
| S8897DNA2LN | LN2 | 0.04 | 0.07 | 0.22 | 0.66 | 0.8 | 0.92 | 1 | 0.32 |
| S8897DNA3LN | LN3 | 0.13 | 0.19 | 0.36 | 0.62 | 0.73 | 0.86 | 1 | 0.36 |
| S8897DNA4LN | LN4 | 0.09 | 0.13 | 0.28 | 0.62 | 0.72 | 0.84 | 1 | 0.33 |
| S8897DNA5LN | LN5 | 0.01 | 0.02 | 0.08 | 0.38 | 0.54 | 0.73 | 0.96 | 0.26 |
| S8897DNA6LN | LN6 | 0.13 | 0.18 | 0.33 | 0.63 | 0.71 | 0.84 | 1 | 0.33 |
| S8897DNA11LN | LN7 | 0.15 | 0.17 | 0.27 | 0.69 | 0.82 | 0.95 | 1 | 0.36 |
| S8897DNA13LN | LN8 | 0.14 | 0.2 | 0.41 | 0.75 | 0.86 | 0.96 | 1 | 0.32 |
| S8897DNA14LN | LN9 | 0.04 | 0.08 | 0.26 | 0.78 | 0.88 | 0.97 | 1 | 0.67 |
| S8897DNA16LN | LN10 | 0.17 | 0.25 | 0.47 | 0.9 | 0.98 | 1 | 1 | 0.4 |
| S8897DNA17LN | LN11 | 0.34 | 0.47 | 0.83 | 1 | 1.04 | 1 | 1 | 0.37 |
| S8897DNA18LN | LN12 | 0.18 | 0.27 | 0.47 | 0.77 | 0.88 | 0.97 | 1 | 0.43 |
| S8897DNA19LN | LN13 | 0.15 | 0.22 | 0.43 | 0.73 | 0.84 | 0.94 | 1 | 0.43 |
| S8897DNA7LN | * | 0.1 | 0.14 | 0.28 | 0.63 | 0.72 | 0.84 | 1 | 0.18 |
| S8897DNA8LN | * | 0.16 | 0.23 | 0.43 | 0.65 | 0.7 | 0.82 | 1 | 0.32 |
| S8897DNA9LN | * | 0 | 0 | 0.02 | 0.44 | 0.63 | 0.84 | 0.99 | 0.27 |
| S8897DNA10LN | ** | | | | | | | | |
| S8897DNA12LN | * | 0.32 | 0.33 | 0.33 | 0.56 | 0.73 | 0.94 | 1 | 0.39 |
| S8897DNA15LN | * | 0 | 0.01 | 0.09 | 0.54 | 0.73 | 0.88 | 1 | 0.36 |
| S8897DNA20LN | * | 0.02 | 0.03 | 0.1 | 0.56 | 0.73 | 0.9 | 1 | 0.25 |

* Tissue depleted.
** Not LN tissue

TABLE 17
UAMS 20 year old Breast Cancer FFPE Tissue Blocks, Tumor RNA Metrics and Yields

| | | | | | | RNA | Qubit | microRNA |
|---|---|---|---|---|---|---|---|---|
| Initial | Final | | | Nanodrop | Qubit | yield by | microRNA | yeild |
| Specimen | Specimen | 260/230 | 260/280 | RNA, | HSRNA, | Qubit, | Assay, | Qubit, |
| ID | ID | (2.00-2.20) | (~2.00) | ng/μl | ng/μl | ng | ng/μl | ng |
| S98-3 | T1 | 1.31 | 1.79 | 49.2 | 34.2 | 1026.0 | 15.1 | 453.0 |
| S98-4 | T2 | 1.56 | 1.93 | 42.4 | 27.8 | 834.0 | 18.0 | 540.0 |
| S98-5 | T3 | 1.53 | 1.93 | 58.0 | 42.4 | 1272.0 | 22.4 | 672.0 |
| S98-6 | T4 | 1.50 | 1.91 | 30.9 | 16.7 | 501.0 | 11.9 | 357.0 |
| S98-8 | T5 | 1.06 | 1.76 | 15.2 | 9.9 | 296.0 | 3.9 | 116.0 |
| S98-9 | T6 | 1.09 | 1.89 | 16.2 | 7.9 | 238.0 | 3.4 | 102.0 |
| S98-11 | T7 | 1.79 | 1.82 | 39.8 | 22.0 | 660.0 | 17.1 | 513.0 |
| S98-12 | T8 | 1.09 | 1.66 | 16.1 | 11.5 | 345.0 | 6.2 | 185.0 |
| S99-7 | T9 | 1.21 | 1.89 | 18.8 | 11.9 | 357.0 | 6.2 | 185.0 |
| S99-16 | T10 | 1.70 | 1.90 | 57.3 | 41.4 | 1242.0 | 18.6 | 558.0 |
| S99-17 | T11 | 1.87 | 1.90 | 118.5 | 75.6 | 2268.0 | 40.6 | 1218.0 |
| S99-18 | T12 | 1.81 | 1.94 | 53.9 | 35.0 | 1050.0 | 17.1 | 513.0 |
| S99-19 | T13 | 1.89 | 1.94 | 82.6 | 58.6 | 1758.0 | 27.2 | 816.0 |

TABLE 18
S8897 Tumor RNA Metrics and Yields

| | | | | | | RNA | Qubit | microRNA |
|---|---|---|---|---|---|---|---|---|
| Initial | Final | | | | | yield by | microRNA | yeild |
| Specimen | Specimen | 260/230 | 260/280 | Nanodrop | Qubit | Qubit, | Assay, | Qubit, |
| ID | ID | (2.00-2.20) | (~2.00) | ng/μl | ng/μl | ng | ng/μl | ng |
| S8897RNA2 | T14 | 1.88 | 2.36 | 21.0 | 12.1 | 363.0 | 8.3 | 206.5 |
| S8897RNA3 | T15 | 1.65 | 2.11 | 35.0 | 25.0 | 750.0 | 15.9 | 397.5 |
| S8897RNA11 | T16 | 1.52 | 2.59 | 35.0 | 22.0 | 660.0 | 14.4 | 360.0 |
| S8897RNA12 | T17 | 1.25 | 1.88 | 10.6 | 1.8 | 43.8 | 4.0 | 99.0 |
| S8897RNA13 | T18 | 1.23 | 2.28 | 23.0 | 10.8 | 324.0 | 10.6 | 265.0 |
| S8897RNA15 | T19 | 0.94 | 2.14 | 30.0 | 14.1 | 423.0 | 12.6 | 315.0 |
| S8897RNA16 | T20 | 1.60 | 2.35 | 45.0 | 24.4 | 732.0 | 13.6 | 340.0 |
| S8897RNA18 | T21 | 1.89 | 3.29 | 41.0 | 27.8 | 834.0 | 21.2 | 530.0 |
| S8897RNA19 | T22 | 1.01 | 3.09 | 15.0 | 5.9 | 175.8 | 6.3 | 158.5 |
| S8897RNA20 | T23 | 2.44 | 2.04 | 23.0 | 10.8 | 324.0 | 9.5 | 238.5 |
| S8897RNA1 | T26 | 1.62 | 2.22 | 106.0 | 61.0 | 1830.0 | 52.2 | 1305.0 |
| S8897RNA4 | T27 | 1.61 | 2.28 | 108.0 | 70.8 | 2124.0 | 47.6 | 1190.0 |
| S8897RNA5 | T28 | 1.39 | 2.00 | 52.0 | 32.4 | 972.0 | 26.4 | 660.0 |
| S8897RNA6 | T29 | 1.77 | 2.25 | 84.0 | 43.6 | 1308.0 | 43.8 | 1095.0 |
| S8897RNA7 | T30 | 1.52 | 2.26 | 58.0 | 36.4 | 1092.0 | 28.8 | 720.0 |
| S8897RNA8 | T31 | 1.45 | 2.40 | 74.0 | 51.6 | 1548.0 | 40.8 | 1020.0 |
| S8897RNA10 | T32 | 1.76 | 2.00 | 118.0 | 79.6 | 2388.0 | 63.2 | 1580.0 |
| S8897RNA14 | T33 | 1.25 | 2.18 | 44.0 | 24.8 | 744.0 | 23.0 | 575.0 |
| S8897RNA9 | * | 1.17 | 1.74 | 11.4 | 7.90 | 237.0 | | |
| S8897RNA17 | ** | 0.80 | 1.72 | 8.2 | 2.35 | 70.6 | | |

* Tissue depleted.
** Not a tumor

TABLE 19
S8897 LN RNA Metrics & Yields

| | | | | | | RNA | Qubit | microRNA |
|---|---|---|---|---|---|---|---|---|
| Initial | Final | | | Nanodrop | Qubit | yield by | microRNA | yield |
| Specimen | Specimen | 260/230 | 260/280 | RNA, | HSRNA | Qubit, | Assay, | Qubit, |
| ID | ID | (2.00-2.20) | (~2.00) | ng/μl | ng/μl | ng | ng/μl | ng |
| S8897RNA1LN | LN1 | 1.24 | 1.90 | 95.0 | 28.4 | 852.0 | 14.2 | 355.0 |
| S8897RNA2LN | LN2 | 1.50 | 1.91 | 173.5 | 32.2 | 966.0 | 16.3 | 407.5 |
| S8897RNA3LN | LN3 | 1.79 | 1.99 | 47.0 | 23.0 | 1150.0 | 22.4 | 560.0 |
| S8897RNA4LN | LN4 | 1.73 | 2.04 | 36.5 | 13.5 | 675.0 | 16.1 | 402.5 |
| S8897RNA5LN | LN5 | 2.17 | 2.25 | 123.0 | 69.2 | 3460.0 | 58.0 | 1450.0 |
| S8897RNA6LN | LN6 | 2.01 | 2.22 | 90.0 | 44.4 | 2220.0 | 40.0 | 1000.0 |
| S8897RNA11LN | LN7 | 1.54 | 2.11 | 34.5 | 8.0 | 238.8 | 7.3 | 183.5 |
| S8897RNA13LN | LN8 | 2.01 | 2.06 | 38.5 | 19.3 | 521.1 | 25.0 | 625.0 |
| S8897RNA14LN | LN9 | 1.66 | 1.88 | 105.5 | 16.3 | 489.0 | 10.4 | 260.0 |
| S8897RNA16LN | LN10 | 1.40 | 1.91 | 76.0 | 9.3 | 277.8 | 8.2 | 204.5 |
| S8897RNA17LN | LN11 | 1.57 | 1.76 | 0.0 | 1.5 | 45.9 | 3.1 | 78.5 |
| S8897RNA18LN | LN12 | 0.60 | 2.69 | 50.0 | 44.0 | 1320.0 | 36.4 | 910.0 |
| S8897RNA19LN | LN13 | 1.46 | 2.62 | 20.5 | 8.0 | 240.6 | 12.0 | 300.0 |
| S8897RNA7LN | * | 1.95 | 2.05 | 58.8 | 26.8 | 1340.0 | 33.6 | 840.0 |
| S8897RNA8LN | * | 2.16 | 2.19 | 113.1 | 53.4 | 2670.0 | 54.8 | 1370.0 |
| S8897RNA9LN | * | 0.95 | 1.91 | 276.5 | 60.4 | 1812.0 | 29.2 | 730.0 |
| S8897RNA10LN | ** | | | | | | | |
| S8897RNA12LN | * | 0.97 | 2.18 | 71.0 | 34.0 | 1020.0 | 36.6 | 915.0 |
| S8897RNA15LN | * | 1.49 | 1.84 | 142.0 | 19.0 | 570.0 | 11.9 | 297.5 |
| S8897RNA20LN | * | 2.00 | 1.97 | 193.2 | 58.2 | 1746.0 | 97.8 | 2445.0 |

* Tissue depleted.
** Not LN tissue

TABLE 20
UAMS 20 year old Breast Cancer FFPE Tissue
Blocks, Tumor RNA Fragment Analysis

| Initial | Final | | | | | | | |
|---|---|---|---|---|---|---|---|---|
| Speci- | Speci- | ≥150 | ≥100 | ≥50 | ≥30 | ≥20 | ≥10 | Peak |
| men ID | men ID | nt | nt | nt | nt | nt | nt | at nt |
| S98-3 RNA | T1 | 0.08 | 0.21 | 0.57 | 0.77 | 0.87 | 0.89 | 40-82 |
| S98-4 RNA | T2 | 0.05 | 0.16 | 0.46 | 0.76 | 0.91 | 0.95 | 34 |
| S98-5 RNA | T3 | 0.03 | 0.11 | 0.40 | 0.73 | 0.90 | 0.96 | 34 |
| S98-6 RNA | T4 | 0.04 | 0.15 | 0.45 | 0.78 | 0.94 | 0.98 | 32 |
| S98-8 RNA | T5 | 0.01 | 0.13 | 0.48 | 0.75 | 0.90 | 0.94 | ~35 |
| S98-9 RNA | T6 | 0.03 | 0.14 | 0.46 | 0.72 | 0.89 | 0.95 | 28-33 |
| S98-11 RNA | T7 | 0.06 | 0.16 | 0.44 | 0.71 | 0.87 | 0.92 | 28-34 |
| S98-12 RNA | T8 | 0.02 | 0.11 | 0.41 | 0.69 | 0.88 | 0.97 | 28-33 |
| S99-7 RNA | T9 | 0.06 | 0.17 | 0.48 | 0.73 | 0.90 | 0.96 | 32 |
| S99-16 RNA | T10 | 0.07 | 0.02 | 0.45 | 0.69 | 0.84 | 0.91 | 24-32 |
| S99-17 RNA | T11 | 0.07 | 0.19 | 0.47 | 0.70 | 0.84 | 0.90 | 33 |
| S99-18 RNA | T12 | 0.08 | 0.23 | 0.53 | 0.72 | 0.84 | 0.90 | 32 |
| S99-19 RNA | T13 | 0.07 | 0.19 | 0.48 | 0.71 | 0.86 | 0.92 | 32 |

TABLE 21
S8897 Tumor RNA Fragmentation Map

| Initial | Final | | | | | | | | % Small |
|---|---|---|---|---|---|---|---|---|---|
| Specimen | Specimen | ≥150 | ≥100 | ≥50 | ≥30 | ≥20 | ≥10 | Peak | RNA |
| ID | ID | nt | nt | nt | nt | nt | nt | at nt | (10-40 nt) |
| S8897RNA2 | T14 | 0.00 | 0.02 | 0.11 | 0.38 | 0.75 | 0.98 | 25 | 85.20% |
| S8897RNA3 | T15 | 0.01 | 0.05 | 0.21 | 0.52 | 0.82 | 0.98 | 27 | 73.60% |
| S8897RNA11 | T16 | 0.02 | 0.06 | 0.22 | 0.53 | 0.82 | 0.98 | 27 | 71.00% |
| S8897RNA12 | T17 | 0.00 | 0.02 | 0.12 | 0.34 | 0.71 | 1.00 | 23 | 84.10% |
| S8897RNA13 | T18 | 0.00 | 0.01 | 0.06 | 0.29 | 0.69 | 0.99 | 23 | 91.50% |
| S8897RNA15 | T19 | 0.01 | 0.02 | 0.06 | 0.38 | 0.79 | 0.99 | 26 | 90.00% |
| S8897RNA16 | T20 | 0.04 | 0.11 | 0.31 | 0.58 | 0.81 | 0.93 | 27 | 61.00% |
| S8897RNA18 | T21 | 0.00 | 0.00 | 0.05 | 0.32 | 0.74 | 0.99 | 25 | 91.60% |
| S8897RNA19 | T22 | 0.00 | 0.01 | 0.05 | 0.25 | 0.67 | 0.98 | 23 | 92.00% |
| S8897RNA20 | T23 | 0.01 | 0.02 | 0.37 | 0.78 | 0.91 | 0.94 | 37 | 49.20% |
| S8897RNA1 | T26 | 0.00 | 0.01 | 0.08 | 0.35 | 0.73 | 0.99 | 25 | 89.00% |
| S8897RNA4 | T27 | 0.02 | 0.05 | 0.23 | 0.62 | 0.87 | 0.98 | 33 | 67.70% |
| S8897RNA5 | T28 | 0.01 | 0.02 | 0.10 | 0.45 | 0.81 | 0.99 | 26 | 84.60% |
| S8897RNA6 | T29 | 0.00 | 0.01 | 0.06 | 0.27 | 0.67 | 0.99 | 23 | 91.80% |
| S8897RNA7 | T30 | 0.01 | 0.02 | 0.08 | 0.41 | 0.78 | 0.99 | 26 | 86.70% |
| S8897RNA8 | T31 | 0.00 | 0.02 | 0.10 | 0.38 | 0.75 | 0.99 | 25 | 85.80% |
| S8897RNA10 | T32 | 0.01 | 0.01 | 0.11 | 0.50 | 0.83 | 0.99 | 27 | 80.40% |
| S8897RNA14 | T33 | 0.01 | 0.02 | 0.06 | 0.38 | 0.79 | 0.99 | 27 | 90.20% |
| S8897RNA9 | * | | | | | | | | |
| S8897RNA17 | ** | | | | | | | | |

* Tissue depleted.
** Not a tumor

TABLE 22
S8897 LN RNA Fragmentation Map

| Initial | Final | | | | | | | | % Small |
|---|---|---|---|---|---|---|---|---|---|
| Specimen | Specimen | ≥150 | ≥100 | ≥50 | ≥30 | ≥20 | ≥10 | Peak | RNA |
| ID | ID | nt | nt | nt | nt | nt | nt | at nt | (10-40 nt) |
| S8897RNA1LN | LN1 | 0.00 | 0.01 | 0.37 | 0.76 | 0.92 | 0.99 | 35 | 51.60% |
| S8897RNA2LN | LN2 | 0.00 | 0.02 | 0.40 | 0.77 | 0.94 | 0.99 | 36 | 49.50% |
| S8897RNA3LN | LN3 | 0.01 | 0.02 | 0.27 | 0.65 | 0.91 | 1.00 | 28 | 64.00% |
| S8897RNA4LN | LN4 | 0.01 | 0.01 | 0.25 | 0.67 | 0.93 | 1.00 | 29 | 65.80% |
| S8897RNA5LN | LN5 | 0.00 | 0.02 | 0.34 | 0.70 | 0.92 | 1.00 | 30 | 58.00% |
| S8897RNA6LN | LN6 | 0.00 | 0.01 | 0.29 | 0.66 | 0.91 | 1.00 | 30 | 62.30% |
| S8897RNA11LN | LN7 | 0.02 | 0.07 | 0.34 | 0.69 | 0.89 | 0.97 | 37 | 55.80% |
| S8897RNA13LN | LN8 | 0.02 | 0.06 | 0.15 | 0.47 | 0.79 | 0.97 | 27 | 79.80% |
| S8897RNA14LN | LN9 | 0.01 | 0.01 | 0.17 | 0.59 | 0.87 | 0.99 | 34 | 73.60% |
| S8897RNA16LN | LN10 | 0.01 | 0.04 | 0.19 | 0.56 | 0.83 | 0.98 | 29 | 73.70% |
| S8897RNA17LN | LN11 | 0.02 | 0.04 | 0.17 | 0.49 | 0.81 | 0.98 | 28 | 78.10% |
| S8897RNA18LN | LN12 | 0.00 | 0.01 | 0.08 | 0.32 | 0.67 | 0.98 | 23 | 88.80% |
| S8897RNA19LN | LN13 | 0.01 | 0.01 | 0.05 | 0.18 | 0.60 | 0.98 | 22 | 94.00% |
| S8897RNA7LN | * | 0.00 | 0.02 | 0.31 | 0.65 | 0.91 | 0.99 | 28 | 61.20% |
| S8897RNA8LN | * | 0.00 | 0.01 | 0.27 | 0.63 | 0.90 | 1.00 | 28 | 65.40% |
| S8897RNA9LN | * | 0.01 | 0.02 | 0.38 | 0.75 | 0.91 | 0.98 | 38 | 49.80% |
| S8897RNA10LN | ** | | | | | | | | |
| S8897RNA12LN | * | 0.00 | 0.00 | 0.10 | 0.46 | 0.85 | 0.98 | 26 | 83.80% |
| S8897RNA15LN | * | 0.01 | 0.01 | 0.26 | 0.67 | 0.89 | 0.99 | 35 | 64.30% |
| S8897RNA20LN | * | 0.02 | 0.06 | 0.17 | 0.47 | 0.80 | 0.97 | 26 | 78.90% |

* Tissue depleted.
** Not LN tissue

From the UAMS cohort (ie, 20-year-old specimens from FFPE blocks), bulk whole transcriptome RNA-seq was performed and was successful (“C” of FIG. 1B). Details of these results are reported in Table 23A. This same RNA-seq approach (“C” of FIG. 1B) was attempted on available S8897 specimens but unsuccessful (“D” of FIG. 1B). In this case, the RNA libraries were successfully built and sequenced however the insert size was found to be too small for mapping by the STAR aligner and Kallisto pseudo-aligner. These mapping attempts included technical recommendations via personnel communications from the tool developers.

TABLE 23A
RNA-seq count data for 11 gene targets and molecular phenotype call data from PAM50, scmod2, and the ddPCR assay

| Initial | Final | | | | | | | | | | | | | scmod2 | | | | |
|---|---|---|---|---|---|---|---|---|---|---|---|---|---|---|---|---|---|---|
| Specimen ID | Specimen ID | ESR1 | PGR | BCL2 | SCUBE2 | ERBB2 | GRB7 | MKI67 | AURKA | BIRC5 | CCNB1 | MYBL2 | PAM50 | subtype | ddPCR | IHCER | IHCPR | IHCHer2 |
| S98-3 | T1 | 1147 | 142 | 5719 | 251 | 2741 | 315 | 8728 | 473 | 109 | 1037 | 2495 | Basal | ER−/ | LumB | Neg | Neg | Neg |
| | | | | | | | | | | | | | | HER2− | | | | |
| | | | | | | | | | | | | | | ER−/ | | | | |
| S98-4 | T2 | 913 | 253 | 5075 | 220 | 2396 | 106 | 5720 | 271 | 81 | 739 | 1727 | Basal | ER−/ | TN | Neg | Neg | Neg |
| | | | | | | | | | | | | | | HER2− | | | | |
| | | | | | | | | | | | | | | ER−/ | | | | |
| S98-5 | T3 | 875 | 108 | 4677 | 184 | 1742 | 136 | 4589 | 198 | 78 | 875 | 988 | Basal | HER2− | TN | Neg | Neg | Neg |
| | | | | | | | | | | | | | | ER−/ | | | | |
| | | | | | | | | | | | | | | HER2− | | | | |
| S98-6 | T4 | 1006 | 594 | 5995 | 153 | 879 | 0 | 0 | 0 | 0 | 438 | 0 | Normal | ER−/ | LumB | Neg | Neg | Neg |
| | | | | | | | | | | | | | | HER2− | | | | |
| | | | | | | | | | | | | | | ER−/ | | | | |
| S98-8 | T5 | 39604 | 1559 | 11318 | 693 | 2225 | 0 | 841 | 60 | 0 | 153 | 144 | LumA | ER+/ | LumA | Pos | Pos | Neg |
| | | | | | | | | | | | | | | HER2− | | | | |
| | | | | | | | | | | | | | | Low Prolif | | | | |
| S98-9 | T6 | 50905 | 3078 | 20939 | 1478 | 2800 | 312 | 794 | 89 | 56 | 288 | 196 | LumA | ER+/ | LumA | Pos | Pos | Neg |
| | | | | | | | | | | | | | | HER2− | | | | |
| | | | | | | | | | | | | | | Low Prolif | | | | |
| S98-11 | T7 | 26930 | 110 | 26331 | 310 | 3502 | 235 | 4559 | 417 | 42 | 579 | 3641 | LumB | ER+/ | LumB | Pos | Neg | Neg |
| | | | | | | | | | | | | | | HER2− | | | | |
| | | | | | | | | | | | | | | High Prolif | | | | |
| S98-12 | T8 | 15420 | 311 | 7839 | 253 | 4392 | 125 | 705 | 0 | 37 | 392 | 366 | LumA | ER+/ | LumA | Pos | Neg | Neg |
| | | | | | | | | | | | | | | HER2− | | | | |
| | | | | | | | | | | | | | | Low Prolif | | | | |
| S99-7 | T9 | 3178 | 0 | 3427 | 1198 | 7145 | 1542 | 529 | 274 | 0 | 809 | 299 | LumA | HER2+ | LumA, | Neg | Neg | Pos |
| | | | | | | | | | | | | | | | Weak Her2 | | | |
| S99-16 | T10 | 497 | 97 | 3644 | 94 | 1566 | 183 | 6756 | 868 | 58 | 1287 | 2521 | Basal | ER−/ | TN | Neg | Neg | Neg |
| | | | | | | | | | | | | | | HER2− | | | | |
| S99-17 | T11 | 1676 | 11 | 2860 | 94 | 2831 | 249 | 7574 | 1407 | 138 | 1221 | 3104 | Basal | ER−/ | TN | Neg | Neg | Neg |
| | | | | | | | | | | | | | | HER2− | | | | |
| S99-18 | T12 | 299 | 24 | 4504 | 0 | 2476 | 80 | 5792 | 1369 | 111 | 1069 | 2667 | Basal | ER−/ | TN | Neg | Neg | Neg |
| | | | | | | | | | | | | | | HER2− | | | | |
| S99-19 | T13 | 502 | 63 | 3179 | 162 | 2456 | 262 | 7448 | 1287 | 69 | 1263 | 2816 | Basal | ER−/ | TN | Neg | Neg | Neg |
| | | | | | | | | | | | | | | HER2− | | | | |

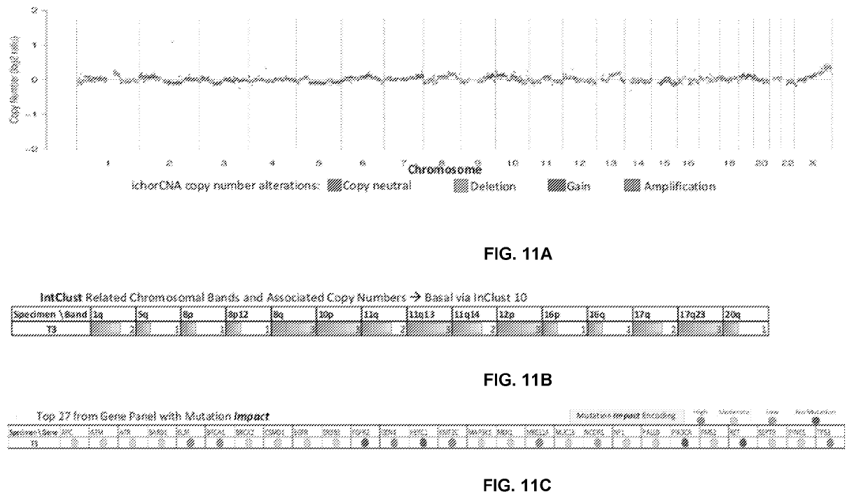

Subsequently, all RNA specimens were subjected to a custom developed ddPCR-based RT-qPCR molecular target quantitation and subtype calling approach (“E” of FIG. 11B). This was successful for all specimens, which included 25 breast tumor specimens (T1-T25) and 13 normal lymph node (LN) specimens. Details of these results are reported in Table 231B. Surplus LN tissue (Final Specimen ID, LN1-LN13) was used for ddPCR assay development. Following ddPCR data normalization, data analytics were performed.

TABLE 23B
Specimens, Molecular Results and Pathology

| | | | | | | | | | Pathologist reading | | | | | |
|---|---|---|---|---|---|---|---|---|---|---|---|---|---|---|
| Initial | Initial | | | | PAM50 Subtype | scmod2 subtype | ddPCR | IntClust | | Invasive, | | % Normal | | |
| Specimen ID | Specimen ID | IHC ER | IHC PR | IHC Her2 | (RNAseq) | (RNAseq) | Subtype | Subtype | Notes | in-situ | % Tumor | Tumor Grade | | Comments |
| S98_3 | T1 | N | N | N | Basal | ER−/ | LumB | Basal | no matched | Inv | 30 | 70 | 3 | invasive ductal |
| | | | | | | HER2− | | | NL tissue | | | | | carcinoma with |
| | | | | | | | | | | | | | | extensive |
| | | | | | | | | | | | | | | inflamation |
| S98_4 | T2 | N | N | N | Basal | ER−/ | TN | Basal | no matched | Inv | 40 | 60 | 3 | invasive ductal |
| | | | | | | HER2− | | | NL tissue | | | | | carcinoma with |
| | | | | | | | | | | | | | | extensive |
| | | | | | | | | | | | | | | inflamation |
| S98_5 | T3 | N | N | N | Basal | ER−/ | TN | Basal | no matched | Inv | 40 | 60 | 3 | invasive ductal |
| | | | | | | HER2− | | | NL tissue | | | | | carcinoma with |
| | | | | | | | | | | | | | | extensive |
| | | | | | | | | | | | | | | inflamation |
| S98_6 | T4 | N | N | N | Normal | ER−/ | LumB | Basal | no matched | Inv | 40 | 60 | 3 | invasive ductal |
| | | | | | | HER2− | | | NL tissue | | | | | carcinoma with |
| | | | | | | | | | | | | | | extensive |
| | | | | | | | | | | | | | | inflamation |
| S98_8 | T5 | P | P | N | LumA | ER+/ | LumA | LumA | no matched | Inv | 70 | 30 | 1 | lobular |
| | | | | | | HER2−, | | | NL tissue | | | | | carcinoma |
| | | | | | | Low Proli | | | | | | | | |
| S98_9 | T6 | P | P | N | LumA | ER+/ | LumA | LumA | no matched | Inv | 60 | 40 | 1 | lobular |
| | | | | | | HER2−, | | | NL tissue | | | | | carcinoma |
| | | | | | | Low Proli | | | | | | | | |
| S98_11 | T7 | P | N | N | LumB | ER+/ | LumB | LumA/B | no matched | Inv | 50 | 50 | 2 | metastatic |
| | | | | | | HER2−, | | | NL tissue | | | | | carcinoma in LN |
| | | | | | | Low Proli | | | | | | | | |
| S98_12 | T8 | P | N | N | LumA | ER+/ | LumA | LumA/B | no matched | Inv | 50 | 50 | 2 | invasive lobular |
| | | | | | | HER2−, | | | NL tissue | | | | | carcinoma with |
| | | | | | | Low Prolif | | | | | | | | necrosis & |
| | | | | | | | | | | | | | | some crush |
| | | | | | | | | | | | | | | artifact |
| S99_7 | T9 | N | N | P | LumA | HER2+ | HER2 | LumA/ | no matched | DCIS | 30 | 70 | 2 | DCIS with |
| | | | | | | | | HER2 | NL tissue | | | | | mucinous |
| | | | | | | | | | | | | | | changes |
| S99_16 | T10 | N | N | N | Basal | ER−/ | TN | Basal | no matched | Inv | 60 | 40 | 3 | invasive ductal |
| | | | | | | HER2− | | | NL tissue | | | | | carcinoma with |
| | | | | | | | | | | | | | | solid pattern |
| S99_17 | T11 | N | N | N | Basal | ER−/ | TN | Basal | no matched | Inv | 80 | 20 | 3 | invasive ductal |
| | | | | | | HER2− | | | NL tissue | | | | | carcinoma with |
| | | | | | | | | | | | | | | solid pattern |
| S99_18 | T12 | N | N | N | Basal | ER−/ | TN | Basal | no matched | Inv | 50 | 50 | 3 | invasive ductal |
| | | | | | | HER2− | | | NL tissue | | | | | carcinoma with |
| | | | | | | | | | | | | | | solid pattern |
| | | | | | | | | | | | | | | and focal clear |
| | | | | | | | | | | | | | | cell features |
| S99_19 | T13 | N | N | N | Basal | ER−/ | TN | Basal | no matched | Inv | 70 | 30 | 3 | invasive ductal |
| | | | | | | HER2− | | | NL tissue | | | | | carcinoma with |
| | | | | | | | | | | | | | | solid pattern |
| | | | | | | | | | | | | | | and focal clear |
| | | | | | | | | | | | | | | cell features |
| Tu2 | T14 | * | * | * | * | * | LumA | LumA | LN2, | Inv | 50 | 50 | 2 | |
| | | | | | | | | | matched NL | | | | | |
| Tu3 | T15 | * | * | * | * | * | LumA | LumA | LN3, | Inv | 95 | 5 | 2 | Comedo in rare |
| | | | | | | | | | matched NL | | | | | DCIS |
| Tu11 | T16 | * | * | * | * | * | Lum A | LumA/B | LN11, | Inv | 15-20 | 80-85 | 2 | Tumor at edge |
| | | | | | | | | | matched NL | | | | | of tissue, fatty |
| | | | | | | | | | | | | | | breast |
| Tu12 | T17 | * | * | * | * | * | NA | NA | Not a tumor | NA | 0 | 100 | NA | Possible rare |
| | | | | | | | | | | | | | | ADH and/or |
| | | | | | | | | | | | | | | ALH |
| Tu13 | T18 | * | * | * | * | * | LumA | LumA | LN13, | DCIS & | 80 (4:1 = | 20 | 1 | tumor necrosis |
| | | | | | | | | | matched NL | Inv | DCIS:Inv) | | | in DCIS, |
| | | | | | | | | | | | | | | comedo |
| Tu15 | T19 | * | * | * | * | * | LumB | LumA | LN15, | Inv | 20 | 80 | 2 | 10-20% |
| | | | | | | | | | matched NL | | | | | immune cells |
| Tu16 | T20 | * | * | * | * | * | LumA | LumA | LN16, | DCIS & | 25 (4:1 = | 75 | 1 | Focal |
| | | | | | | | | | matched NL | Inv | DCIS:Inv) | | | calcifications in |
| | | | | | | | | | | | | | | DCIS |
| Tu18 | T21 | * | * | * | * | * | | | LN18, | Inv | <5 | >95 | 2 | Scant tumor at |
| | | | | | | | | | matched NL | | | | | the edge of the |
| | | | | | | | | | | | | | | tissue |
| Tu19 | T22 | * | * | * | * | * | LumA | LumA | LN19, | Inv | 100 | 0 | 1 | |
| | | | | | | | | | matched NL | | | | | |
| Tu20 | T23 | * | * | * | * | * | HER2 | HER2 | LN20, | DCIS & | 50 (4:1 = | 50 | 2 | Tumor in 1 out |
| | | | | | | | | | matched NL | Inv | DCIS:Inv) | | | of 3 pieces |
| | | | | | | | | | | | | | | (biggest piece) |
| MCF7 | T24 | * | * | * | * | * | Lum B | * | control | NA | 100 | 0 | NA | MCF-7 cell line |
| | | | | | | | | | for ddPCR | | | | | |
| MB-231 | T25 | * | * | * | * | * | TN, | * | control | NA | 100 | 0 | NA | MDA-MB-231 |
| | | | | | | | weak HER2 | | for ddPCR | | | | | cell line |
| Tu1 | T26 | * | * | * | * | * | RNA | LumA | RNA consumed | Inv | 90 | 10 | 1 | |
| | | | | | | | not avail | | in early test | | | | | |
| Tu4 | T27 | * | * | * | * | * | RNA | LumA | RNA consumed | Inv | 100 | 0 | 2 | microcalcificatins |
| | | | | | | | not avail | | in early test | | | | | in tumor |
| Tu5 | T28 | * | * | * | * | * | RNA | LumA | RNA consumed | Inv | 60 | 40 | 1 | |
| | | | | | | | not avail | | in early test | | | | | |
| Tu6 | T29 | * | * | * | * | * | RNA | LumA | RNA consumed | Inv & | 95 | 5 | 2 | |
| | | | | | | | not avail | | in early test | focal | | | | |
| | | | | | | | | | | DCIS | | | | |
| Tu7 | T30 | * | * | * | * | * | RNA | LumA/B | RNA consumed | Inv & | 40 (4:1 = | 60 | 3 | tumor necrosis |
| | | | | | | | not avail | | in early test | DCIS | Inv:DCIS) | | | in DCIS |
| Tu8 | T31 | * | * | * | * | * | RNA | LumB | RNA consumed | Inv | 30 | 70 | 3 | 70-80% |
| | | | | | | | not avail | | in early test | | | | | immune cells |
| Tu10 | T32 | * | * | * | * | * | RNA | LumB | No matched LN; | Inv & | 100 (9:1, | 0 | 2 | |
| | | | | | | | not avail | | RNA consumed | DCIS | Inv:DCIS) | | | |
| Tu14 | T33 | * | * | * | * | * | RNA | LumA | RNA consumed | DCIS | 15 | 85 | 2 | |
| | | | | | | | not avail | | in early test | | | | | |
| LN1 | LN1 | * | * | * | * | * | NA (LN) | * | tissue group | NA | 0 | NA | NA | NL lymph node |
| | | | | | | | | | for ddPCR | | | | | (LN) |
| LN2 | LN2 | * | * | * | * | * | NA (LN) | * | tissue group | NA | 0 | NA | NA | INL LN |
| | | | | | | | | | for ddPCR | | | | | |
| LN3 | LN3 | * | * | * | * | * | NA (LN) | * | tissue group | NA | 0 | NA | NA | NL LN |
| | | | | | | | | | for ddPCR | | | | | |
| LN4 | LN4 | * | * | * | * | * | NA (LN) | * | tissue group | NA | 0 | NA | NA | INL LN |
| | | | | | | | | | for ddPCR | | | | | |
| LN5 | LN5 | * | * | * | * | * | NA (LN) | * | tissue group | NA | 0 | NA | NA | NL LN |
| | | | | | | | | | for ddPCR | | | | | |
| LN6 | LN6 | * | * | * | * | * | NA (LN) | * | tissue group | NA | 0 | NA | NA | NL LN |
| | | | | | | | | | for ddPCR | | | | | |
| LN11 | LN7 | * | * | * | * | * | NA (LN) | * | tissue group | NA | 0 | NA | NA | NL LN |
| | | | | | | | | | for ddPCR | | | | | |
| LN13 | LN8 | * | * | * | * | * | NA (LN) | * | tissue group | NA | 0 | NA | NA | NL LN |
| | | | | | | | | | for ddPCR | | | | | |
| LN14 | LN9 | * | * | * | * | * | NA (LN) | * | tissue group | NA | 0 | NA | NA | NL LN |
| | | | | | | | | | for ddPCR | | | | | |
| LN16 | LN10 | * | * | * | * | * | NA (LN) | * | tissue group | NA | 0 | NA | NA | NL LN |
| | | | | | | | | | for ddPCR | | | | | |
| LN17 | LN11 | * | * | * | * | * | NA (LN) | * | tissue group | NA | 0 | NA | NA | NL LN |
| | | | | | | | | | for ddPCR | | | | | |
| LN18 | LN12 | * | * | * | * | * | NA (LN) | * | tissue group | NA | 0 | NA | NA | NL LN |
| | | | | | | | | | for ddPCR | | | | | |
| LN19 | LN13 | * | * | * | * | * | NA (LN) | * | tissue group | NA | 0 | NA | NA | NL LN |
| | | | | | | | | | for ddPCR | | | | | |

* denotes the assay was not run.
Abbreviatons:
Inv, Invasive;
NA, Not Applicable;
N, Negative;
NL, Normal;
P, Positive
The following specimens (tumor blocks) were from the same patients: T1-4; T5-6; T10-13

### Example 2: Visualizing the Breast Tumor Subtype

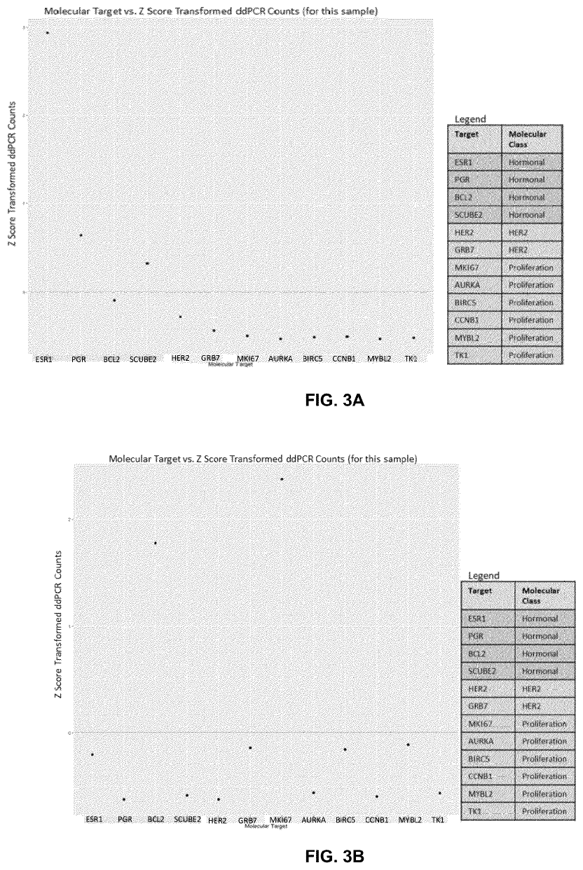

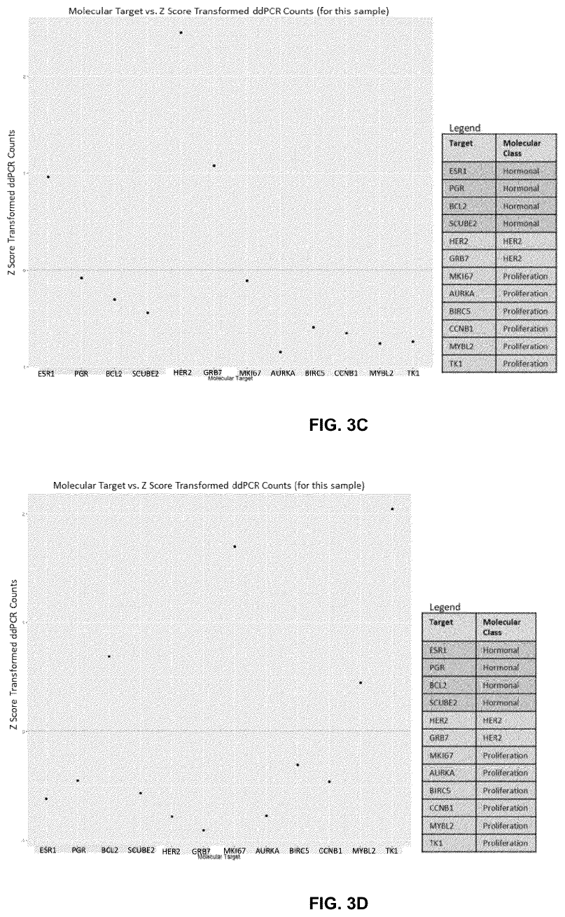

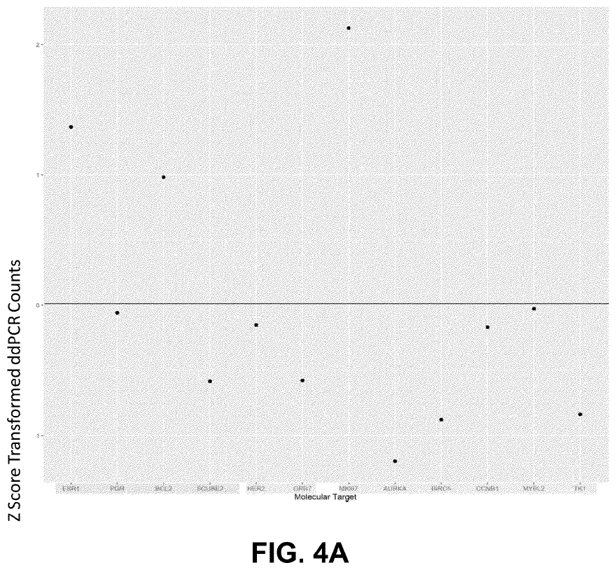

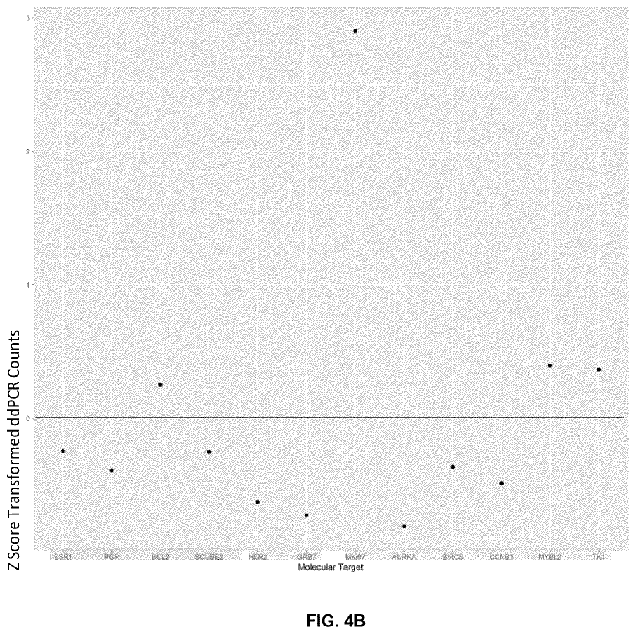

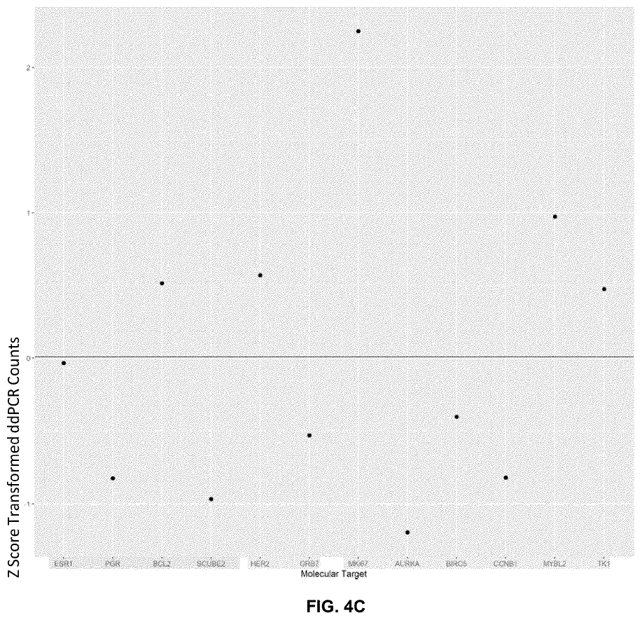

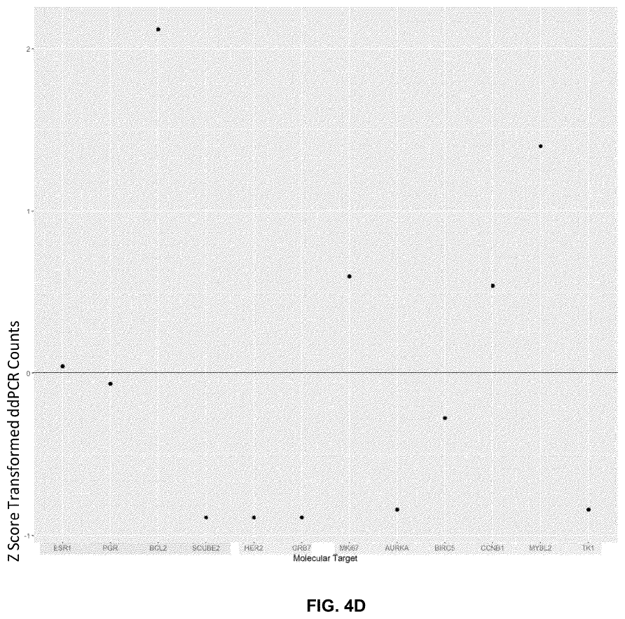

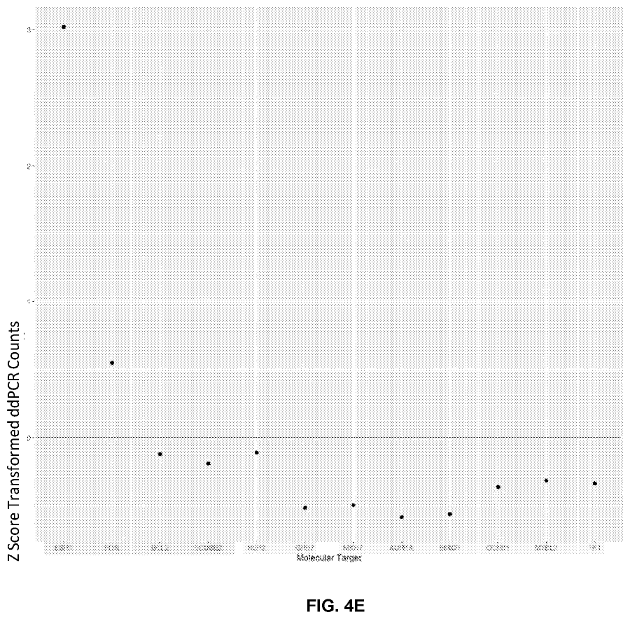

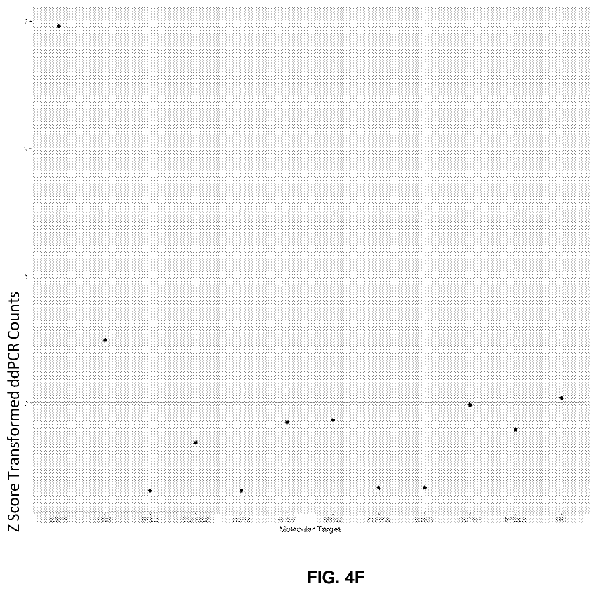

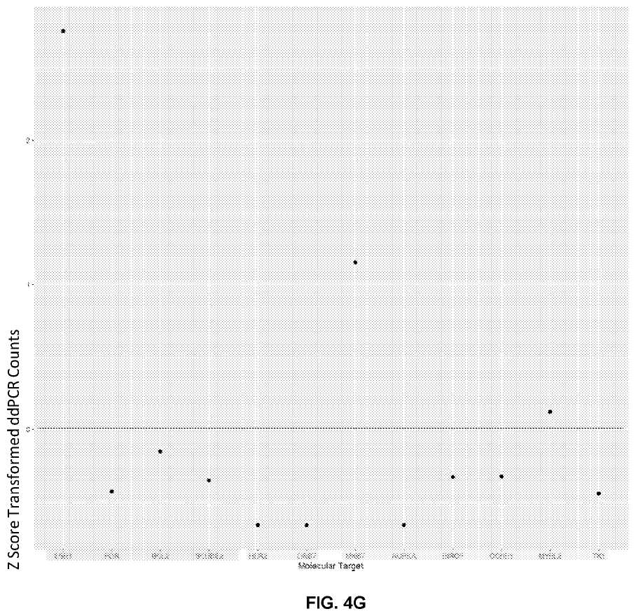

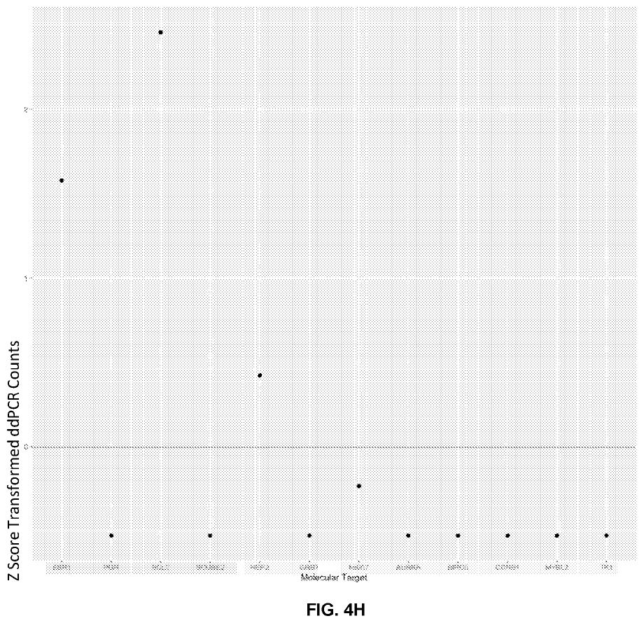

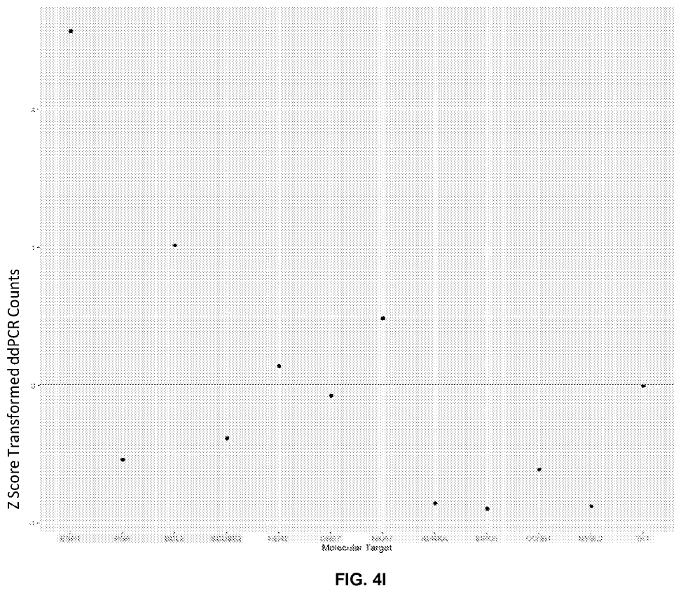

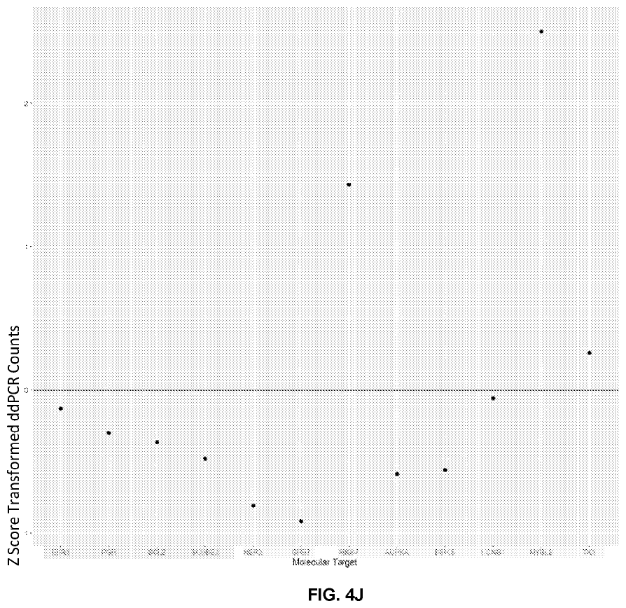

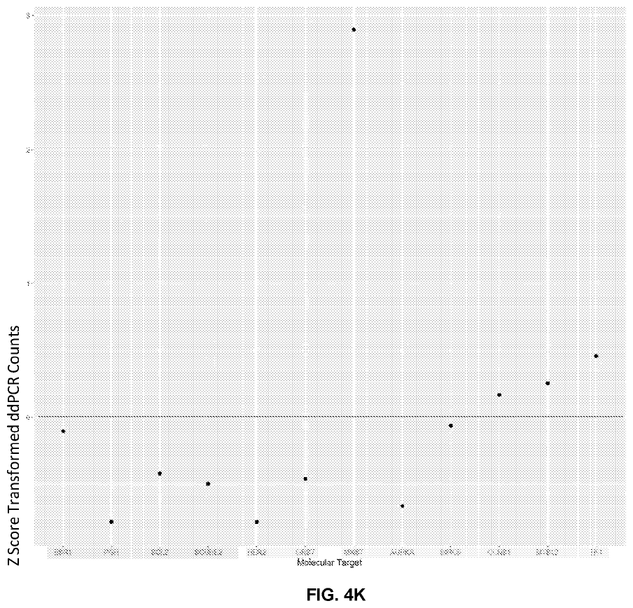

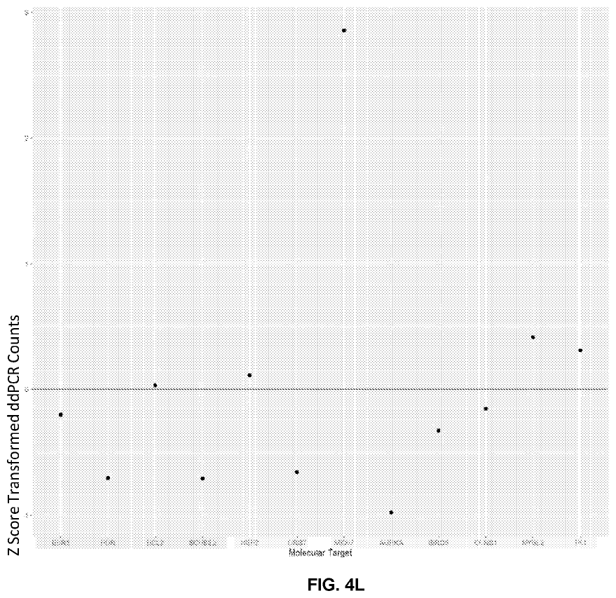

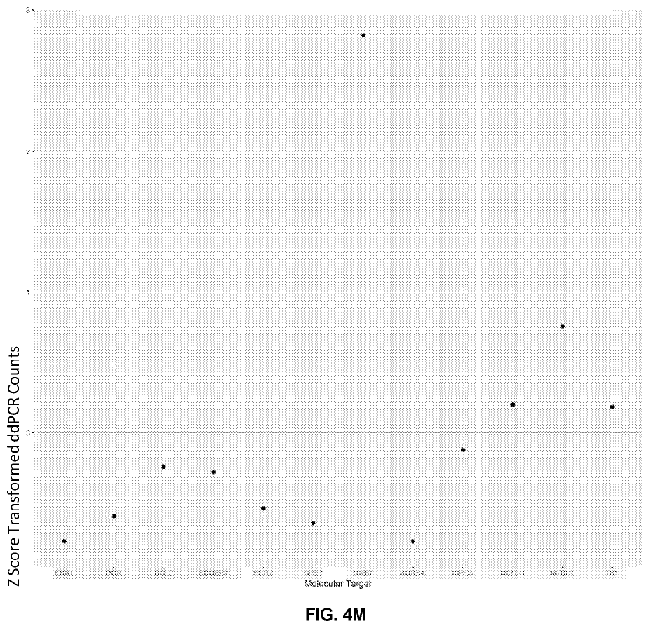

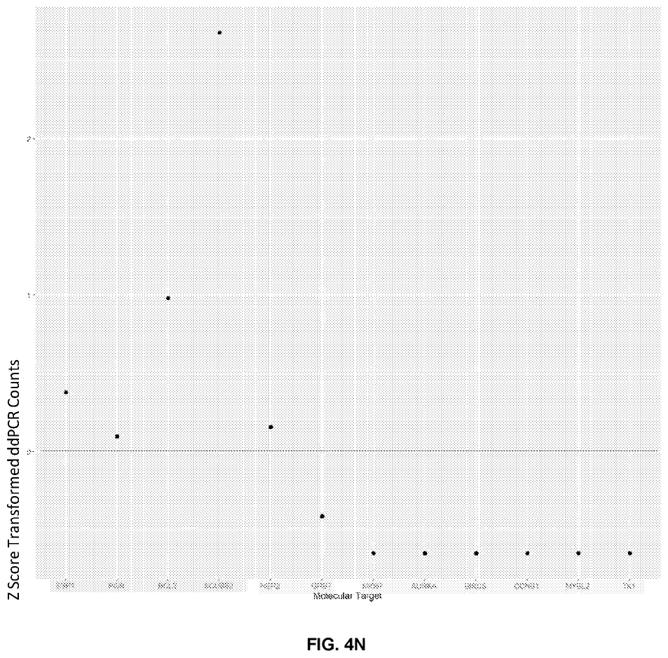

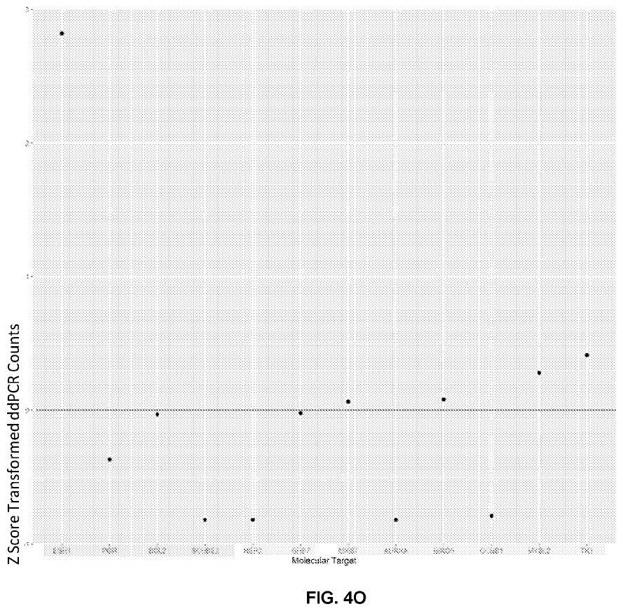

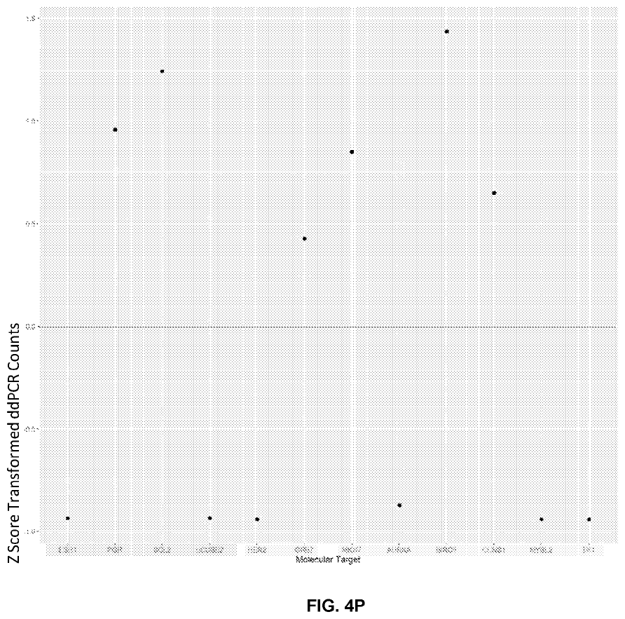

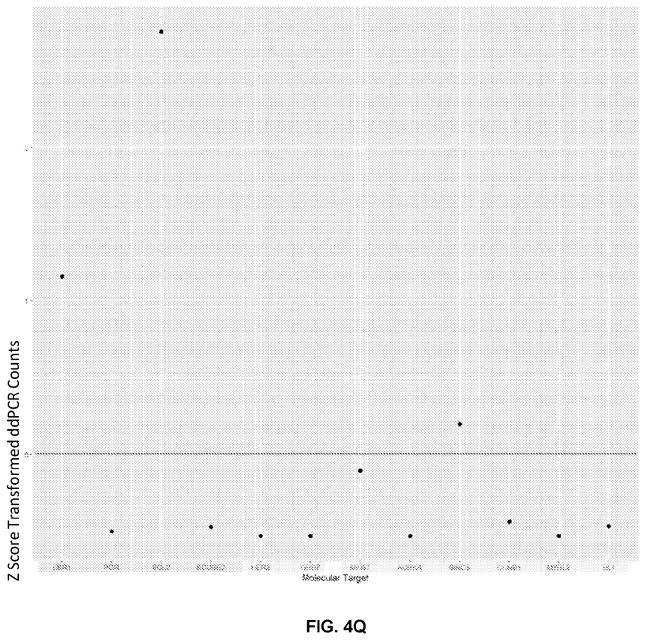

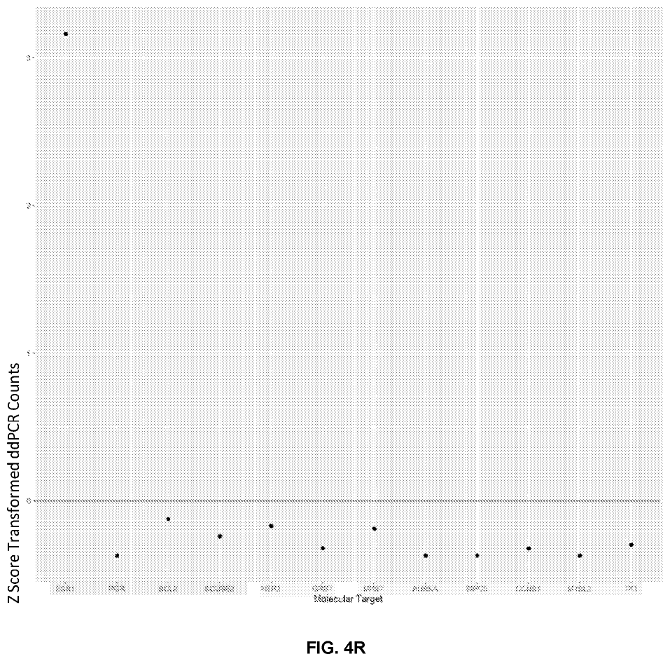

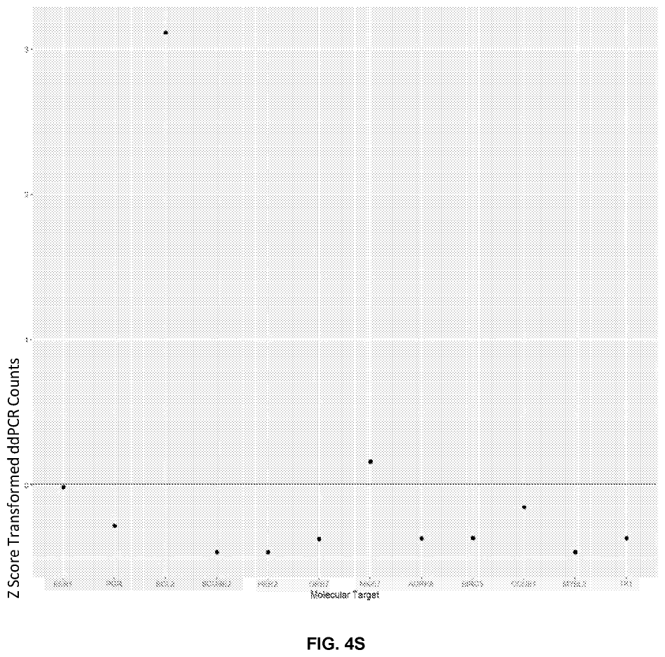

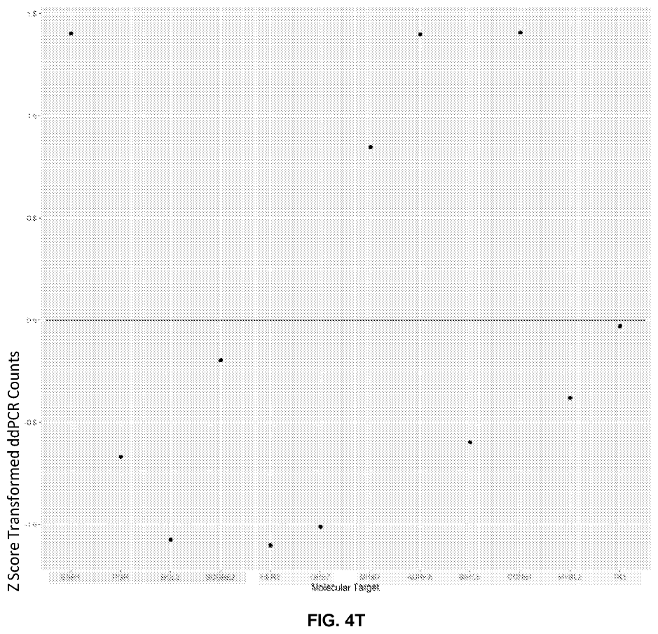

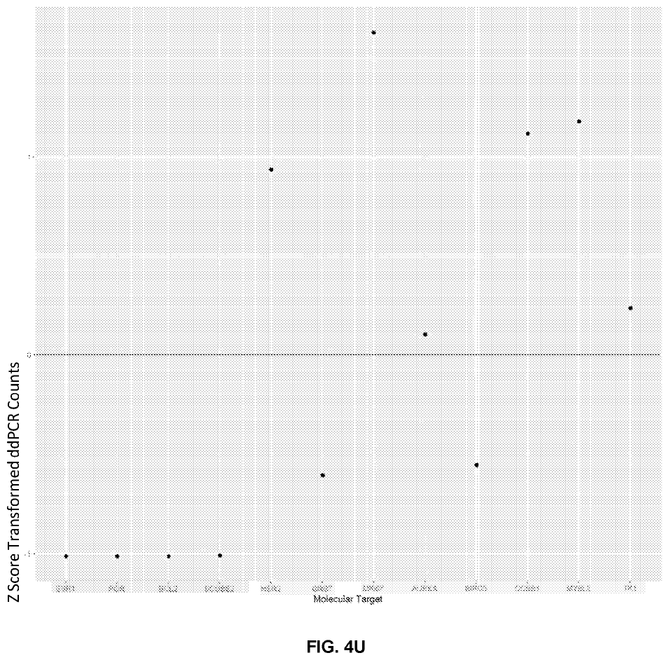

A custom assay visualization technique was developed for RNA tumor specimens and utilized for determining the breast cancer tumor subtype (FIG. 3A-3D, FIG. 4A-4U, and Table 24. A composite of the four breast tumor subtypes is illustrated in FIG. 3A-3D. In each case, the x-axis contains the molecular targets and the y-axis the z-score transform of ddPCR counts. Hormonal targets are colored blue and represented by ESR1, PGR, BCL2, and SCUB2. HER2 targets are colored orange and represented by HER2 and GRB7. Proliferation targets are colored green and represented by MK167, AURKA, BIRC5, CCNB1, MYBL2, and TK1. A molecular target is considered elevated if the z-score is 1. Luminal A (Lum A) subtype (“A” of FIG. 1B) is defined by having only one or more hormonal targets elevated. In this case specimen T15 was classified as Lum A. An example of a Luminal B (Lum B) subtype (“B” of FIG. 1B) is defined by having one or more hormonal and proliferation targets elevated. In this case specimen T19 was classified as Lum B. A HER2 subtype (“C” of FIG. 1) was defined by having only one or more HER2 targets elevated and specimen T23 was classified as HER2. An example of a Triple Negative (TN) subtype (“D” of FIG. 1B) was defined by having only one or more proliferation targets elevated, and specimen T21 was classified as TN. The assay visualization technique was applied to the tumor samples including UAMS 20 Year Old Breast Tumors (Cut from FFPE Blocks, Specimen IDs T1-T13), and S8897 Breast Tumors (20-30 years old, cut onto glass slides, Specimen IDs T14-T23), and Breast Cancer Cell Lines as positive controls (Specimen IDs T24 (MCF-7 cell line) & T25 (MDA-MB-231 cell line)), and breast cancer tumor subtype was determined as shown in Table 24.

TABLE 24
ddPCR calls for specimen T1-T25

| Specimen ID | ddPCR call | FIG. No. |
|---|---|---|
| T1 | Lum B | FIG. 4A |
| T2 | TN | FIG. 4B |
| T3 | TN | FIG. 4C |
| T4 | Lum B | FIG. 4D |
| T5 | Lum A | FIG. 4E |
| T6 | Lum A | FIG. 4F |
| T7 | Lum B | FIG. 4G |
| T8 | Lum A | FIG. 4H |
| T9 | Lum A | FIG. 4I |
| T10 | TN | FIG. 4J |
| T11 | TN | FIG. 4K |
| T12 | TN | FIG. 4L |
| T13 | TN | FIG. 4M |
| T14 | Lum A | FIG. 4N |
| T15 | Lum A | FIG. 3A |
| T16 | Lum A | FIG. 4O |
| T17 | NA | FIG. 4P |
| T18 | Lum A | FIG. 4Q |
| T19 | Lum B | FIG. 3B |
| T20 | Lum A | FIG. 4R |
| T21 | TN | FIG. 3D |
| T22 | Lum A | FIG. 4S |
| T23 | Her2 | FIG. 3C |
| T24 | Lum B | FIG. 4T |
| T25 | TN, weak Her2 | FIG. 4U |

FIG. 3E further shows RNA-seq count data for 11 gene targets along with molecular phenotype call data from PAM50, scmod2, and the ddPCR assay. The RNA-seq, PAM50 and scmod2 assays were all run during the same time period and used tissue recuts in close proximity. The ddPCR assay was performed at a later date and used tissue recuts from different areas of the FFPE blocks or different blocks from the case. Due to tissue heterogeneity, assays using tissue from different areas of a tissue block may experience both technical and biological variation within the tumor tissue. The column Sample Name contains the 13 breast cancer samples from the 20-year-old UAMS cohort. The next four columns (ESR1, PGR, BCL2, SCUBE2) colored blue are the gene symbols of the estrogen-related molecular markers. The next two columns (ERBB2, GRB7 [orange]) are gene symbols of the Her2 enriched columns, and the next five columns (MKI67, AURKA, BIRC5, CCNB1, MYBL2 [green]) are the gene symbols of the proliferation markers. The final three columns contain results from analyses by PAM50, scmod2, and the ddPCR assay. The integer values listed under the estrogen, Her2 enriched, and proliferation markers are from unnormalized RNA-seq count data, where all samples were run in the same RNA-seq assay.

### Example 3: Distance Geometry Analysis

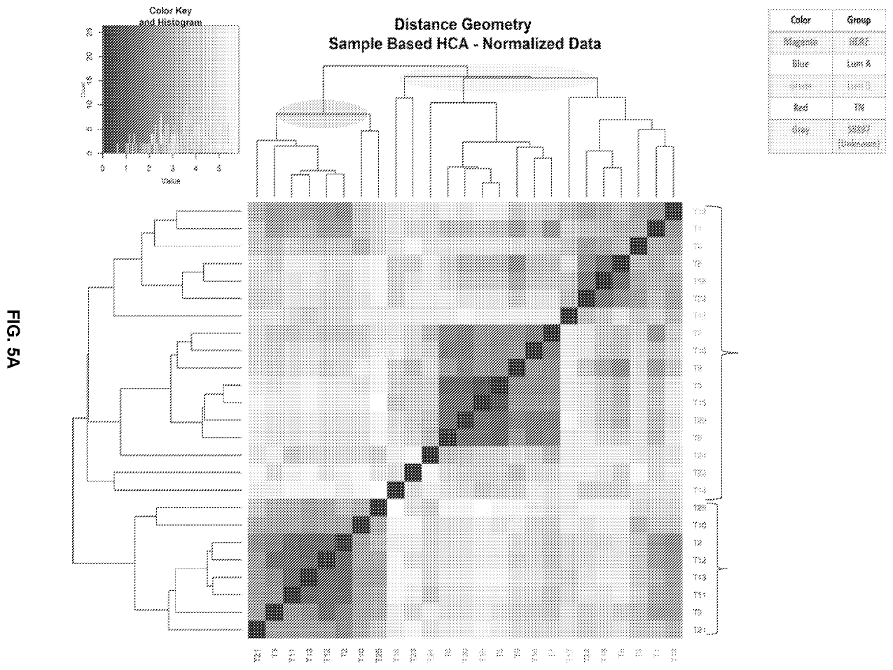

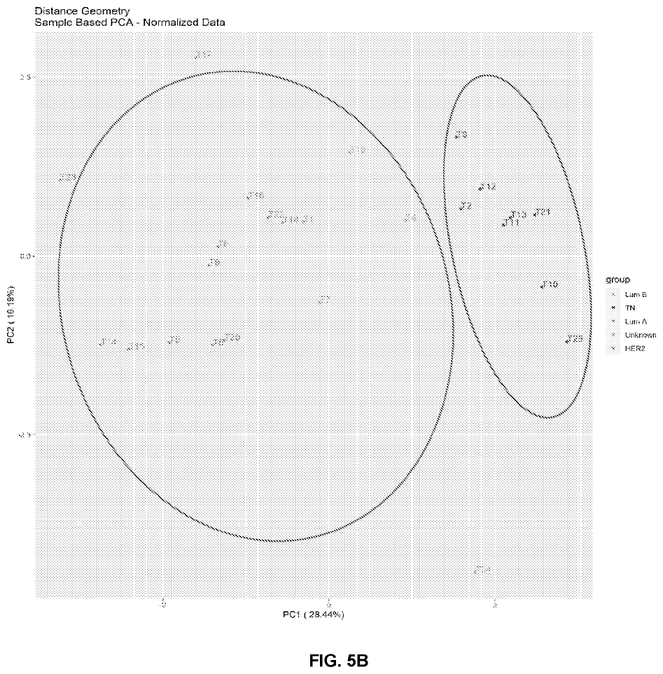

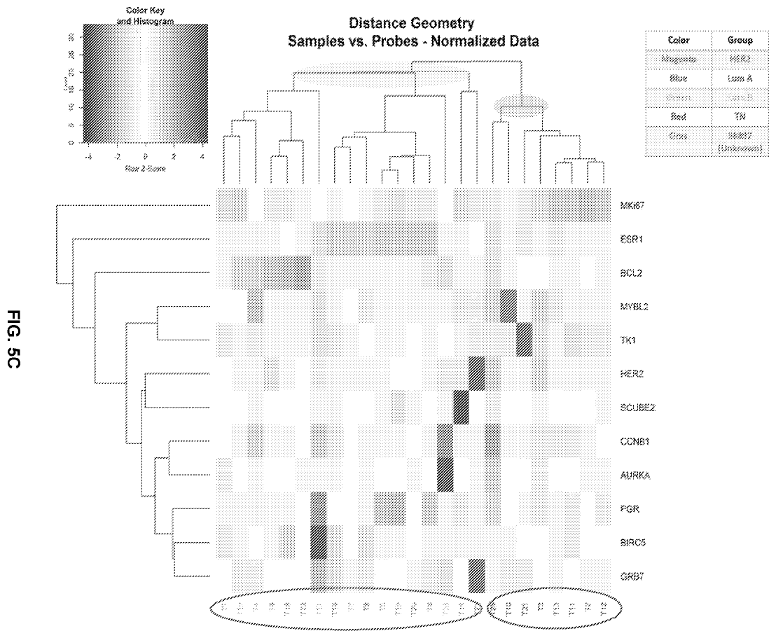

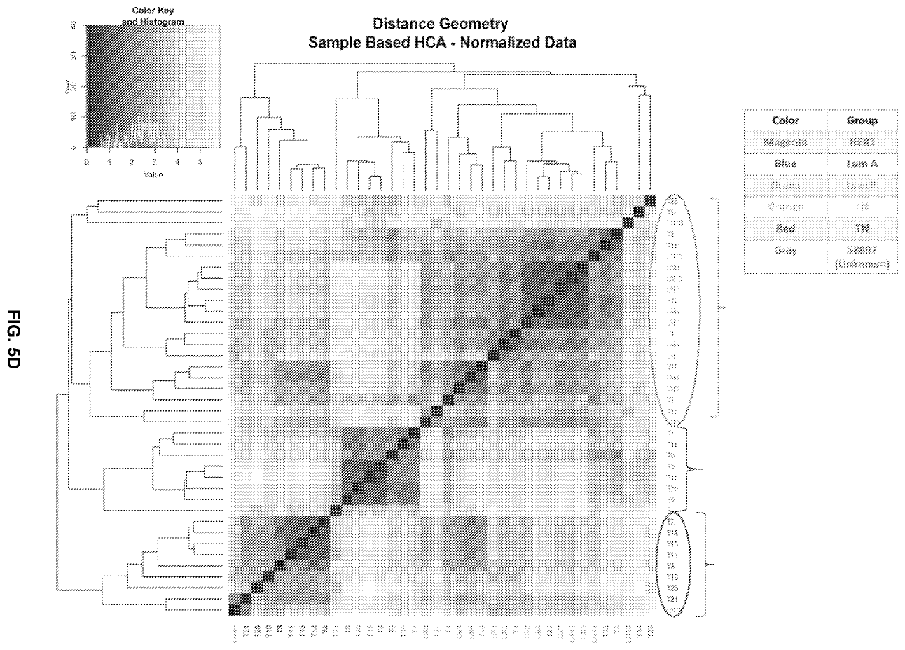

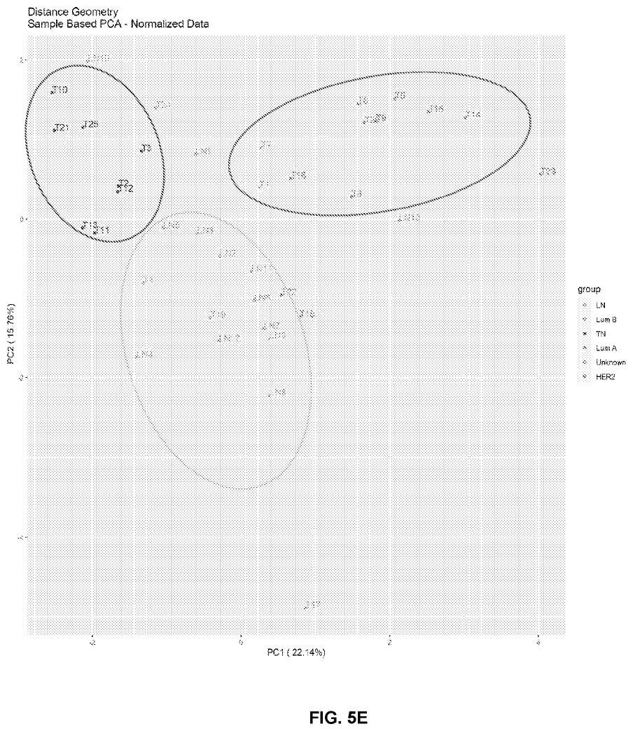

Distance geometry studies (FIG. 5A-5F) were performed to verify an unsupervised clustering on a breast tumor subtype basis (ie, Lum A, Lum B, HER2, TN) and in some circumstances, a tissue basis to determine if the ddPCR assay would distinguish LN tissue from breast tumors. These analyses also included the target types of molecular classes (ie, Hormonal, HER2, or Proliferation) used in breast tumor subtype classification. The results of the three distance geometry analyses were reported for specimens T1-T25. FIG. 5A displays a specimen-based HCA of the 25 breast cancer specimens and shows good grouping of the Luminal cohort vs. the Triple Negative (TN) cohort. FIG. 5B shows the same 25 specimens following PCA and again showed good segregation of the Luminal specimens (blue circle) vs. Triple Negative (red). FIG. 5C illustrates the HCA of the same 25 breast cancer specimens on the x-axis versus the 12 molecular markers used by the ddPCR assay (for breast cancer molecular subtype determination) on the y-axis. This analysis also showed good discrimination of the Luminal-based specimens vs the Triple Negative (TN) specimens.

FIG. 5D-5F show the results of 25 breast tumor specimens (T1-T25) along with the addition of 13 normal human lymph node specimens (LN1-LN13) from the S8897 cohort following processing and analysis by the ddPCR assay. Like the S8897 breast cancer specimens, these lymph node specimens were also unstained FFPE material mounted on glass microscope slides and stored with no particular special processing or storage conditions for over 30 years. Analysis using distance geometry methods, e.g., HCA and PCA involving 38 specimens (T1-T25, LN1-LN13) specimens follows. FIG. 5D displays a specimen-based HCA of these 38 specimens and which showed good grouping of the following cohorts: i) Triple Negative (TN), ii) Luminal and iii) a cohort composed of 12/13 lymph nodes along with some of the Luminal A and B specimens. FIG. 5E shows the same 38 specimens following PCA and again showed good segregation of the Luminal specimens (blue circle) vs. TN (red) vs. Lymph Node (LN, yellow) with a few of the Luminal A and B specimens. FIG. 5F illustrates the HCA of the 38 specimens on the x-axis versus the 12 molecular markers on the y-axis. This analysis also showed good discrimination of the TN breast cancer specimens (red circle) vs Luminal (blue) vs Lymph Node with scattered Luminal A and B specimens (yellow).

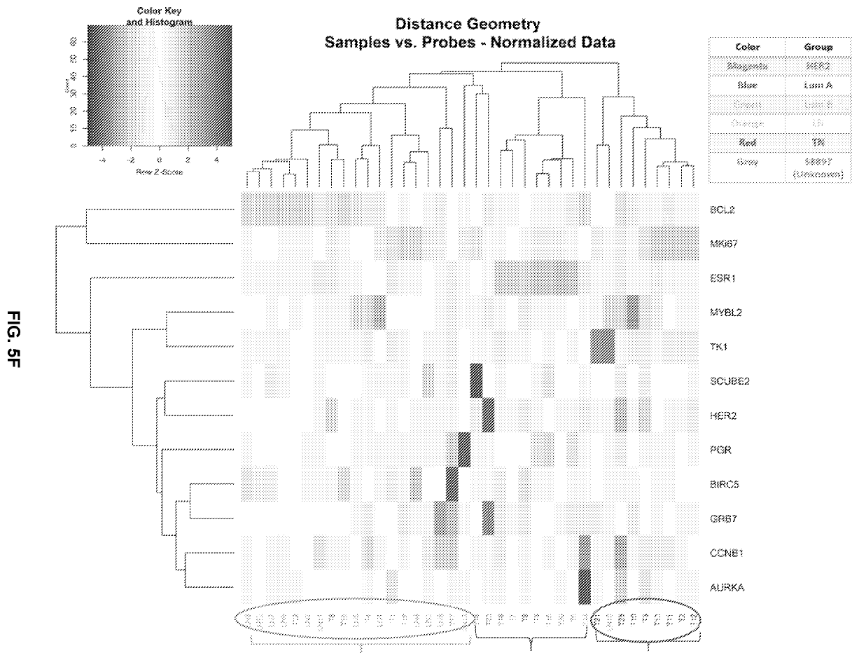

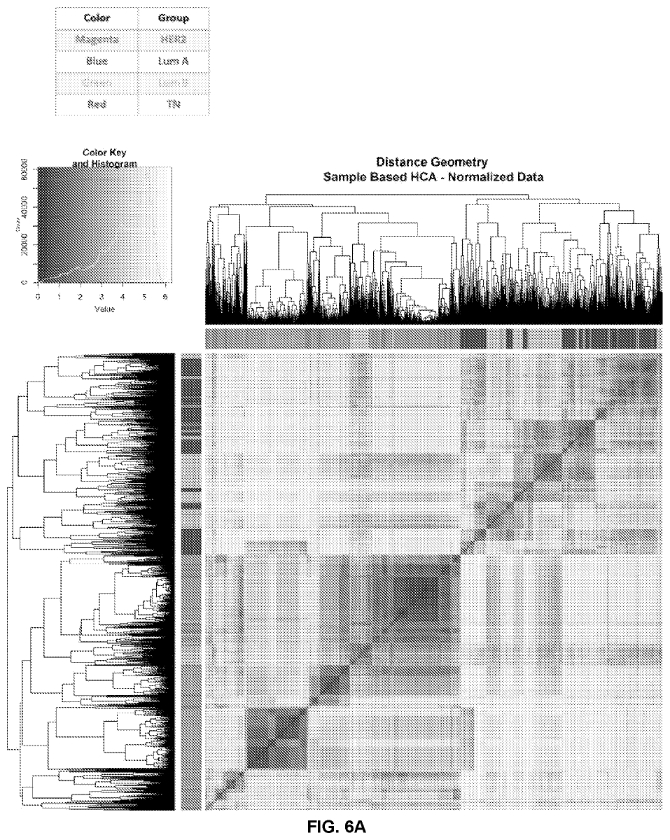

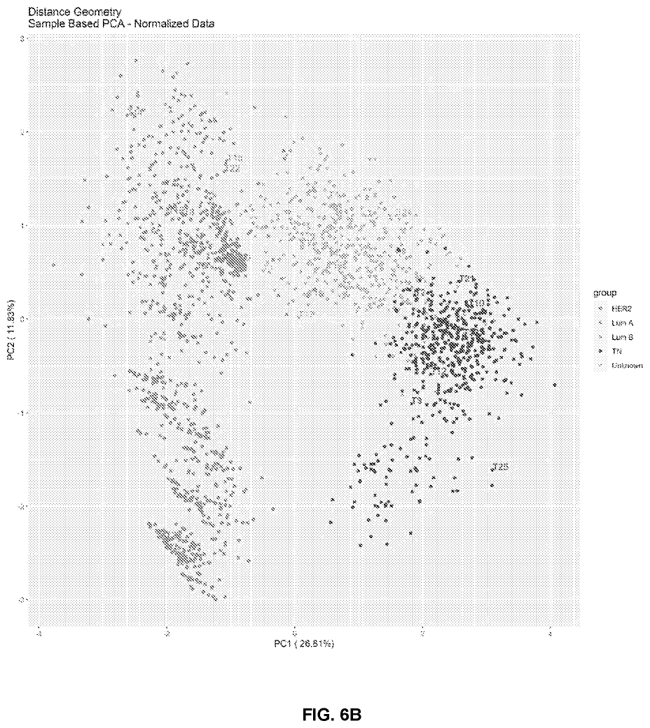

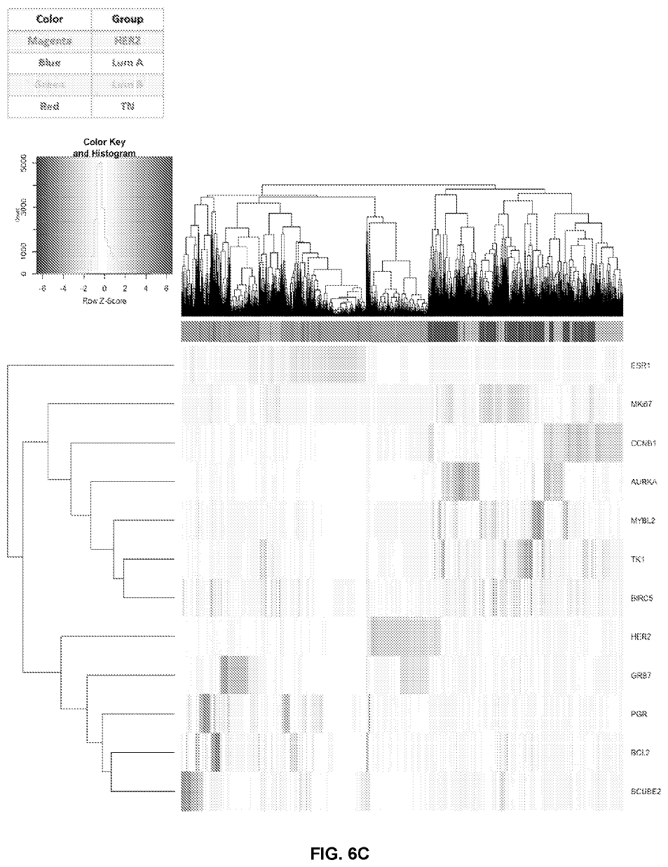

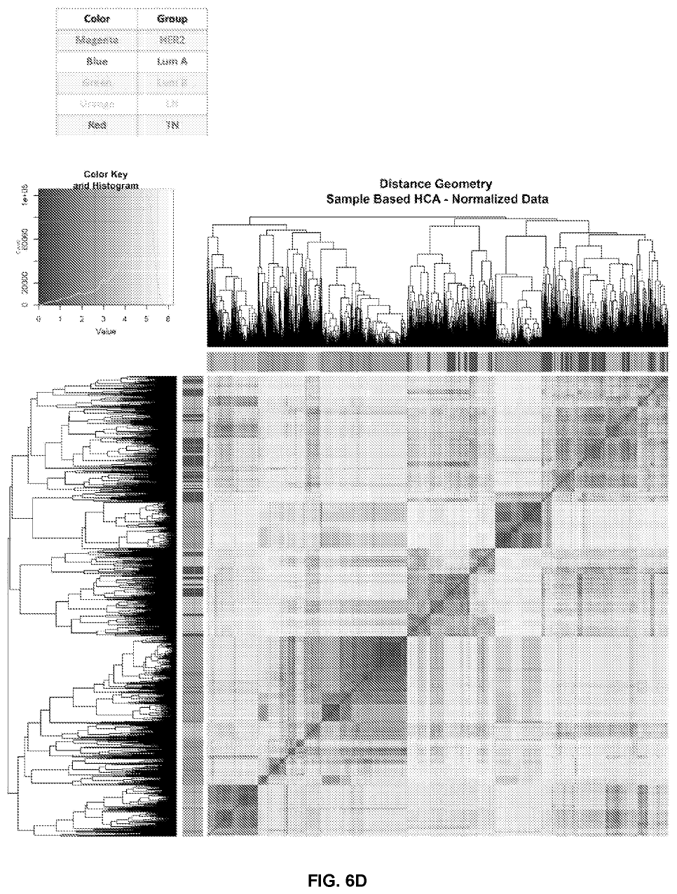

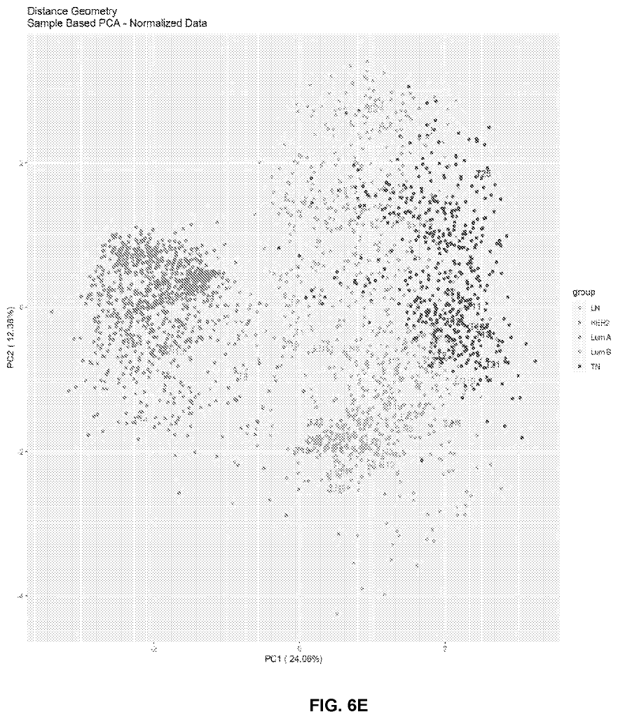

Statistical simulation was performed to derive more robust estimates through additional distance geometry studies of the actual experimental specimens (FIG. 6A-6F). FIG. 6A-6C show the results of tumor specimens (T1-T25) analysis using distance geometry methods (HCA, PCA) with statistical simulation via non parametric bootstrap and generating 500 synthetic specimens from ddPCR assay data for each PAM50 subtype. FIG. 6A displays a specimen-based HCA of the actual (T1-T25) and synthetic breast cancer specimens and showed good grouping and discrimination of all PAM50 cohorts (Lum A, Lum B, Her2, TN). FIG. 6B shows the results of PCA involving the 25 actual breast cancer specimens along 500 synthetic specimens for each PAM50 subtype, and there was good segregation of the PAM50 subtypes. FIG. 6C illustrates the HCA of the actual 25 breast cancer specimens with 500 synthetic specimens for each PAM50 subtype on the x-axis versus the 12 molecular markers used by the ddPCR assay (for breast cancer molecular subtype determination) on the y-axis. This analysis also showed good discrimination of the four PAM50 subtypes (Lum A, Lum B, Her2, TN).

FIG. 6D-6F show the results of tumor and lymph node specimens (T1-T25, LN1-LN13) analysis using distance geometry methods (HCA, PCA) with statistical simulation via non parametric bootstrap and generating 500 synthetic specimens from ddPCR assay data for each PAM50 subtype and 500 synthetic normal lymph node specimens. FIG. 6D displays a specimen-based HCA of the 38 actual specimens and synthetic specimens and showed good grouping of all PAM50 cohorts (Lum A, Lum B, Her2, TN) and lymph nodes. FIG. 6E shows the results of PCA involving the 38 actual specimens along 500 synthetic specimens for each PAM50 subtype and lymph node, and there was good segregation of the PAM50 subtypes lymph nodes. Finally, FIG. 6F illustrates the HCA of the actual 38 specimens with 500 synthetic specimens for each PAM50 subtype and lymph node on the x-axis versus the 12 molecular markers used by the ddPCR assay (for breast cancer molecular subtype determination) on the y-axis. This analysis also showed good discrimination of the four PAM50 subtypes (Lum A, Lum B, Her2, TN) and normal lymph node.

### Example 4: DNA Specimens

All DNA specimens (“F” of FIG. 1B) were subjected to NGS analysis using the matched normal LN as a germline comparison, if available. DNA analyses were done in a tumor-only fashion if LN tissue was not available. Tumor specimens T1-T13 (UAMS cohort) and T32 (S8897 cohort) did not have a matched LN. Regarding specimens T1-T13, although there is a total of 13 tumor block specimens, this tissue was derived from six unique patients. Specifically, T1-T4 were four tumor blocks from the same patient, T5 and T6 were two tumor blocks from a unique patient, and T10-T13 were four tumor blocks from a unique patient. The large amount of tissue provided adequate material for assay development including, sensitivity, accuracy, reproducibility, cross correlation with IHC and, and RNA-seq vs ddPCR RT-qPCR comparisons. The use of these specific specimen types and assay approaches were also useful for assessing tumor heterogeneity, since it is established that high genetic diversity may exist in a single solid tumor.

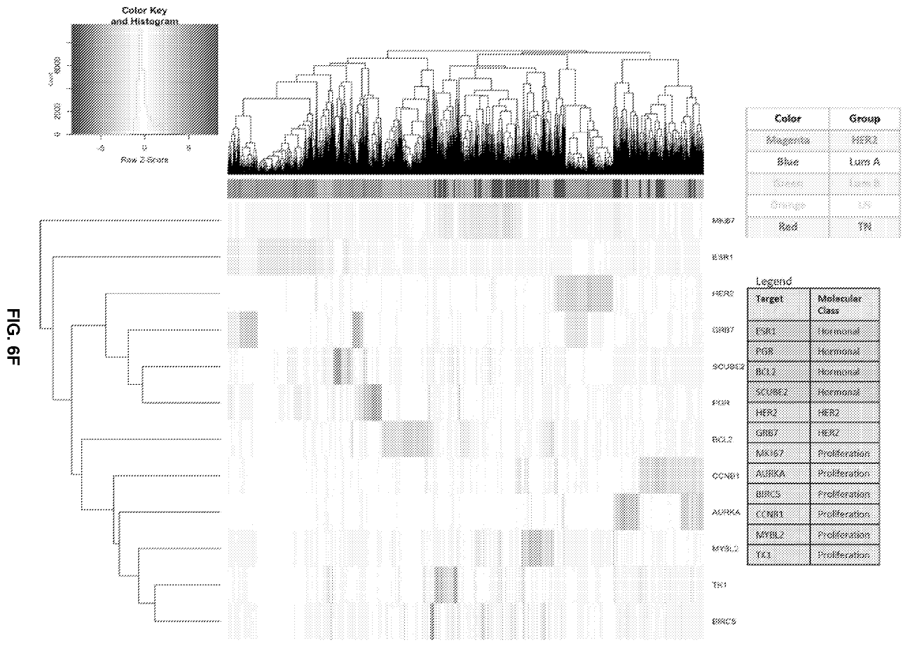

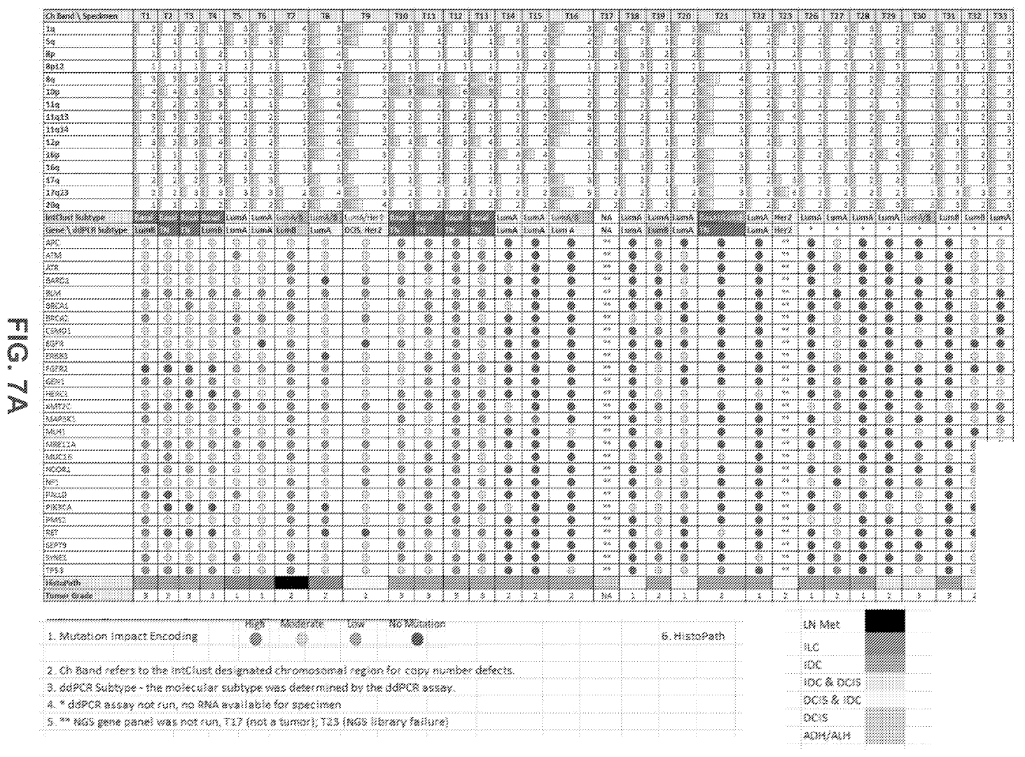

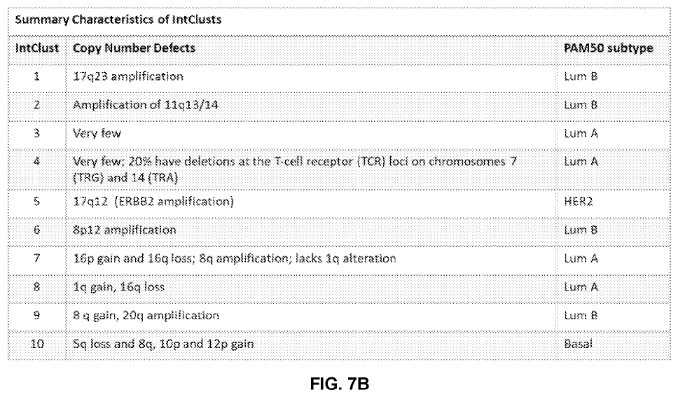

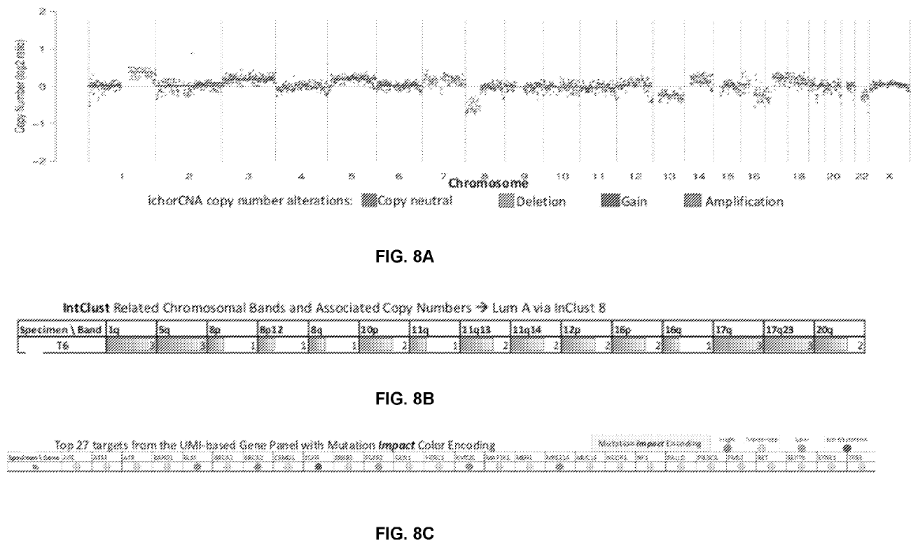

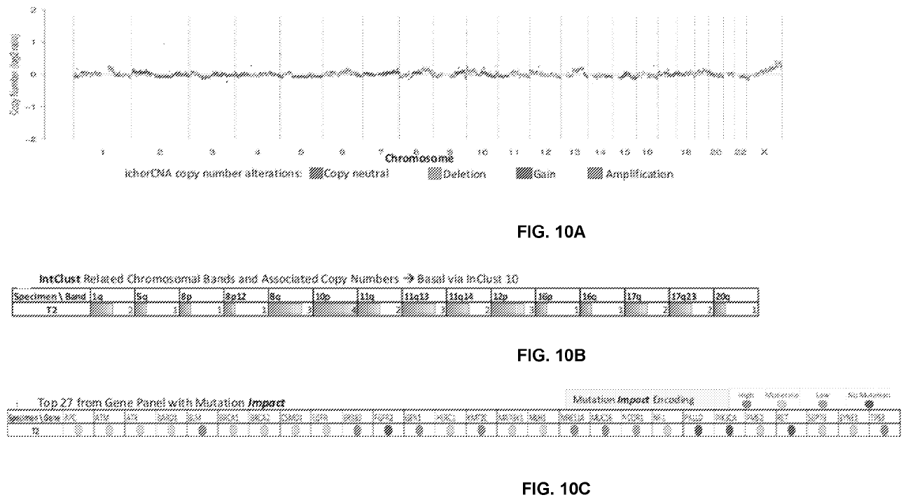

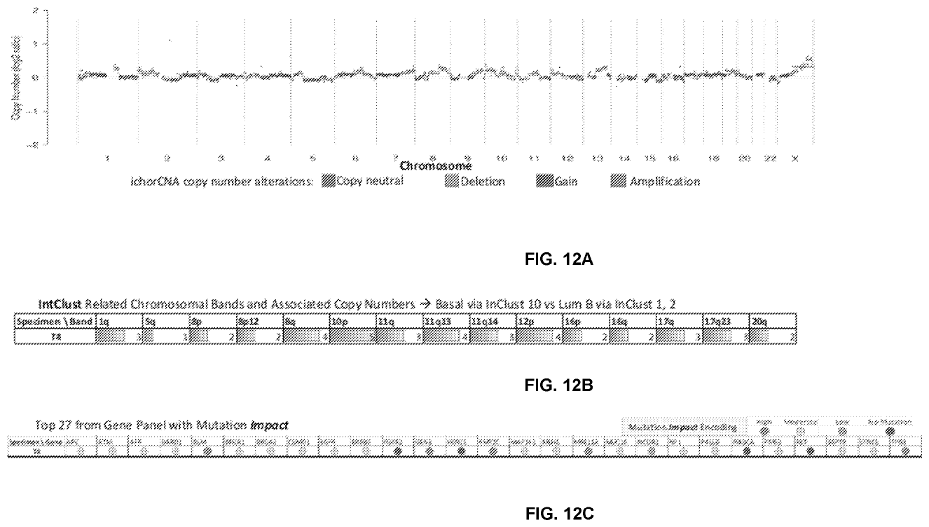

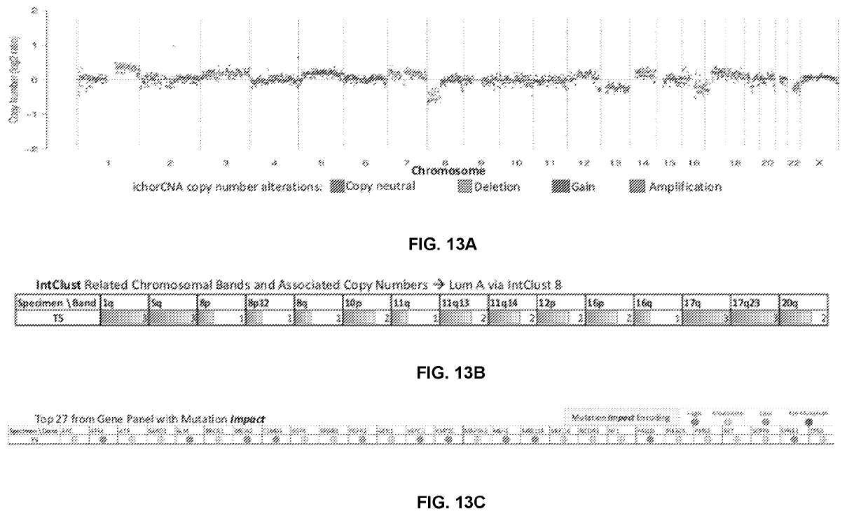

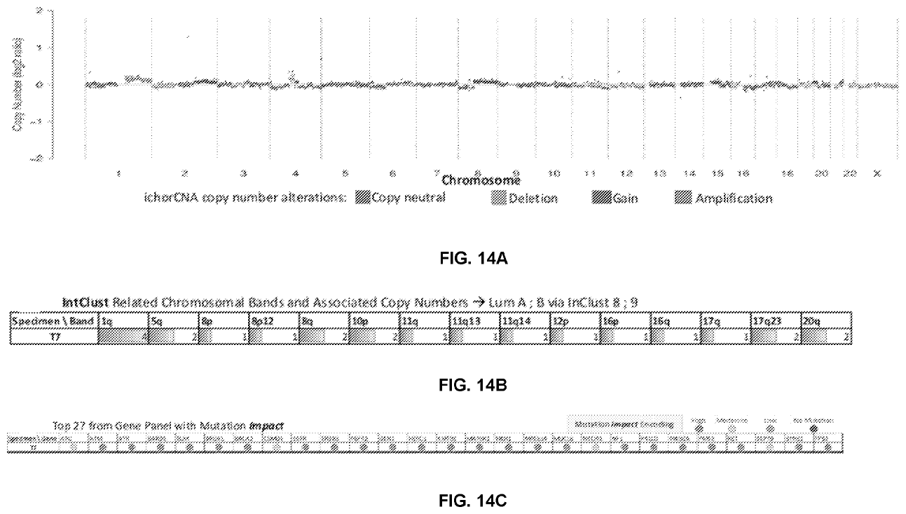

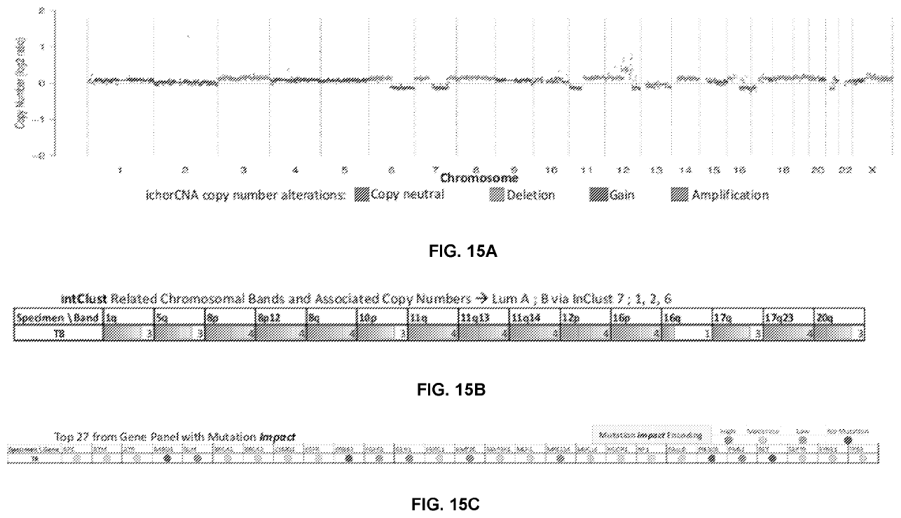

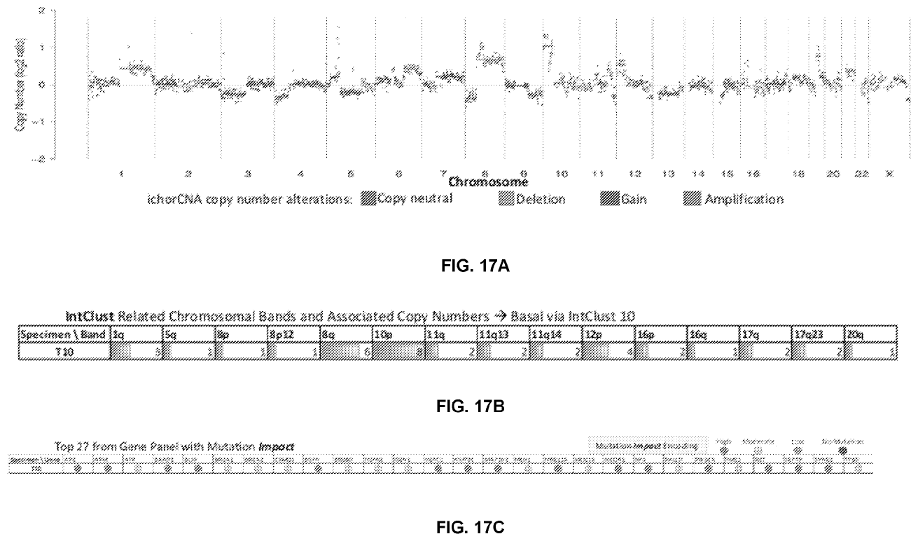

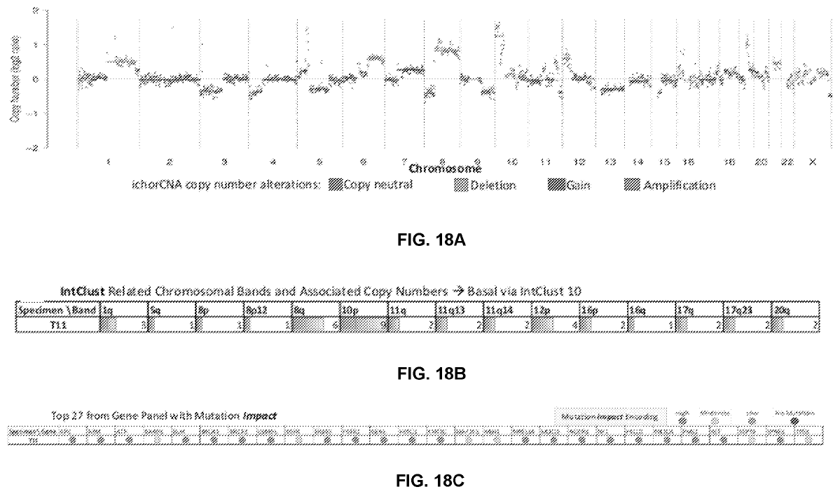

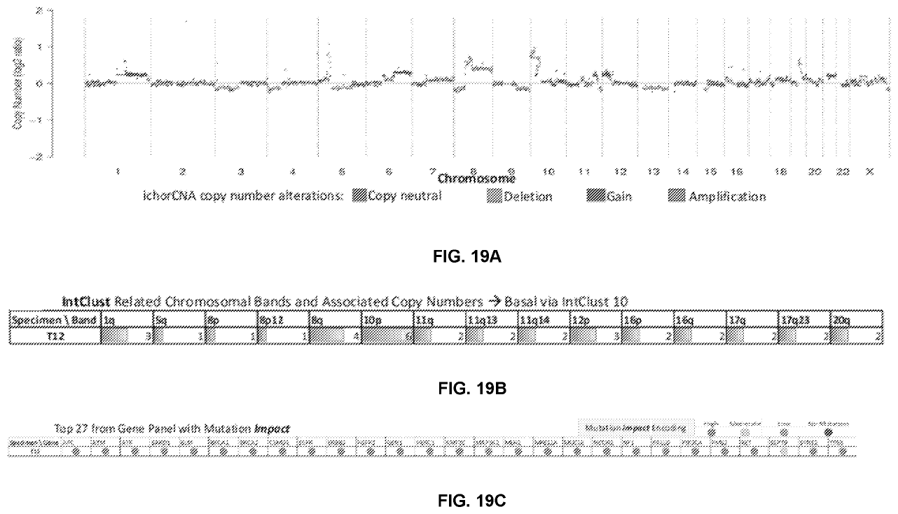

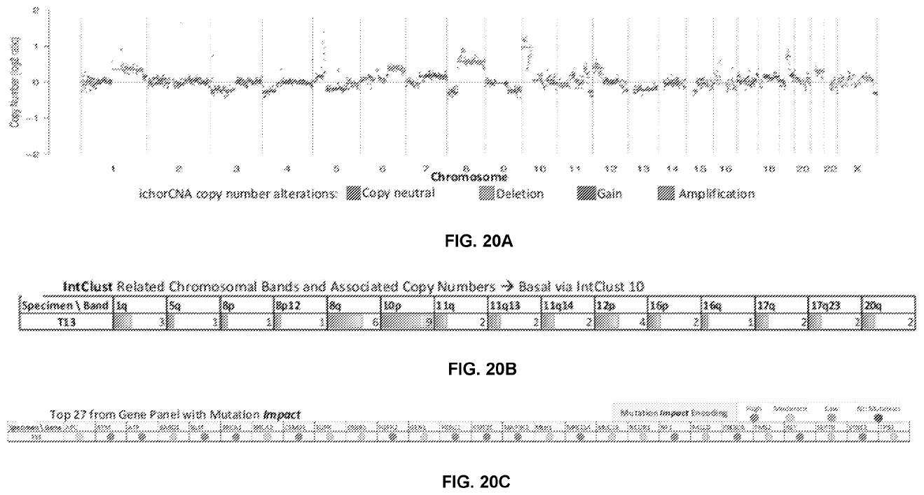

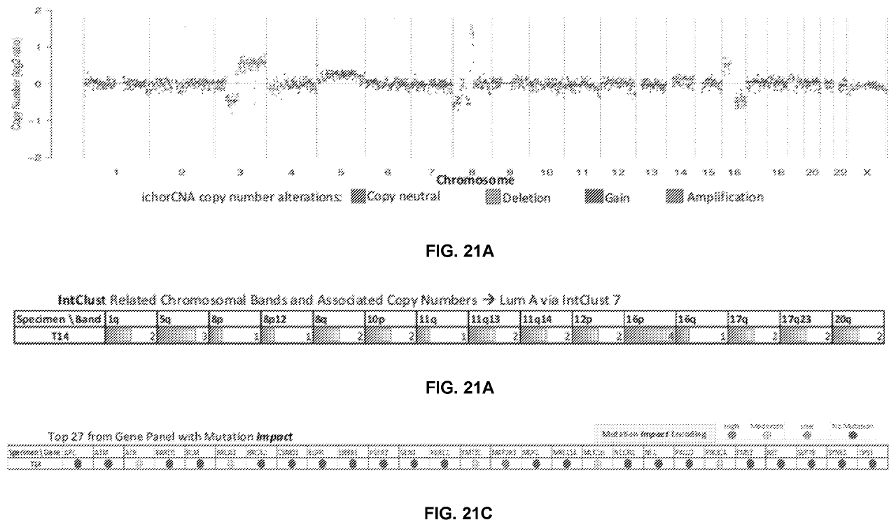

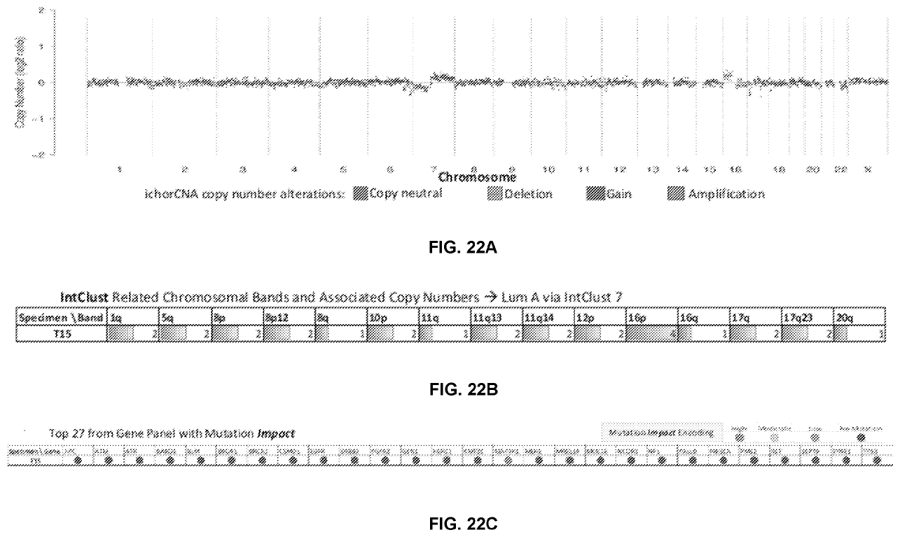

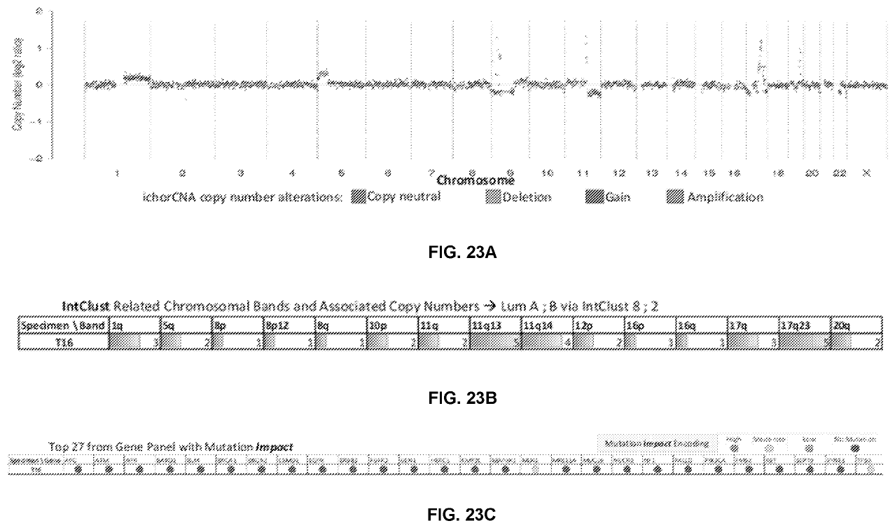

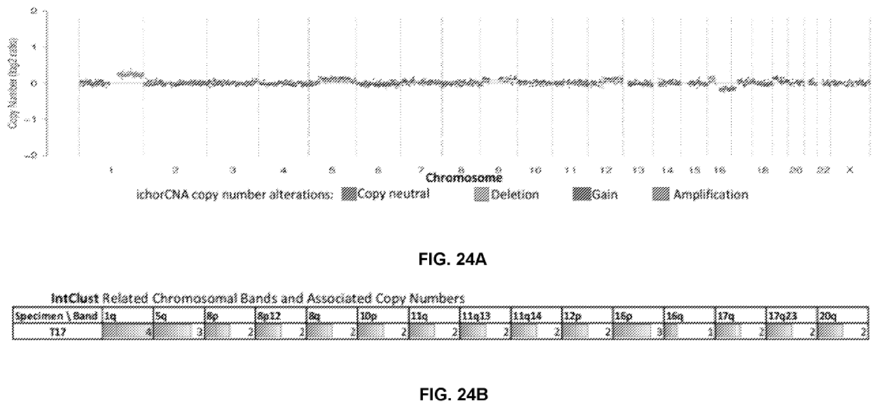

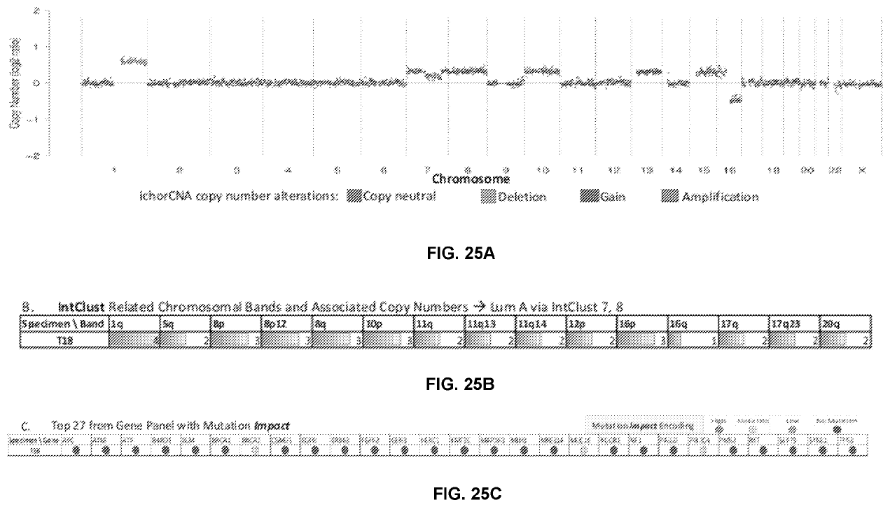

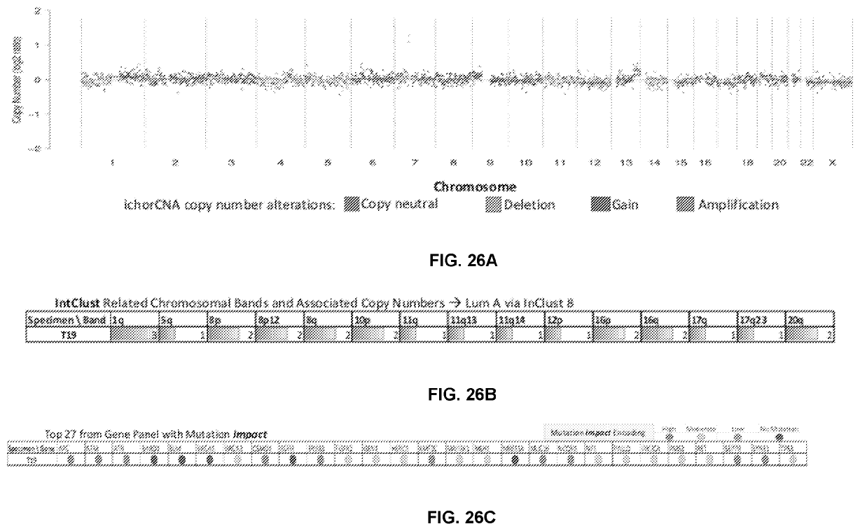

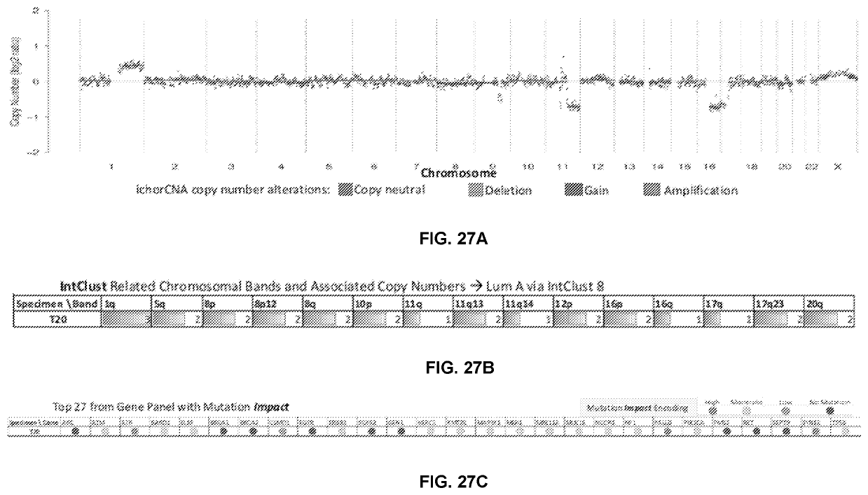

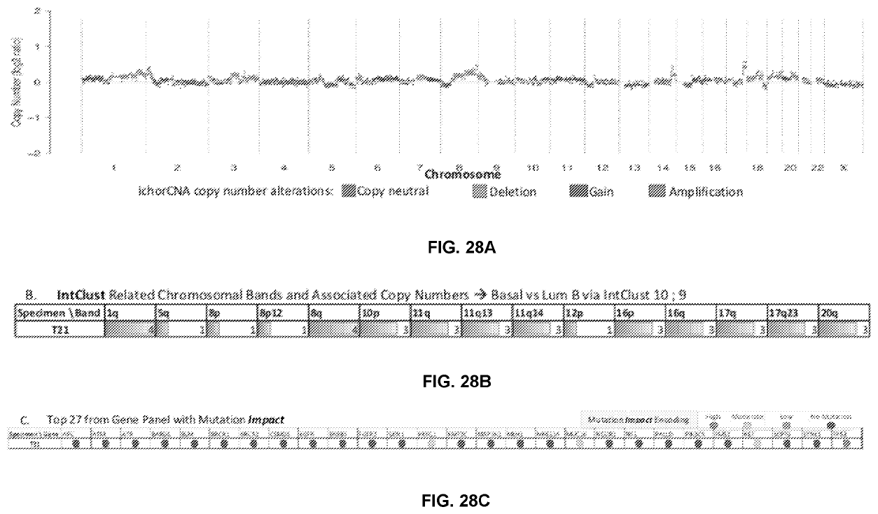

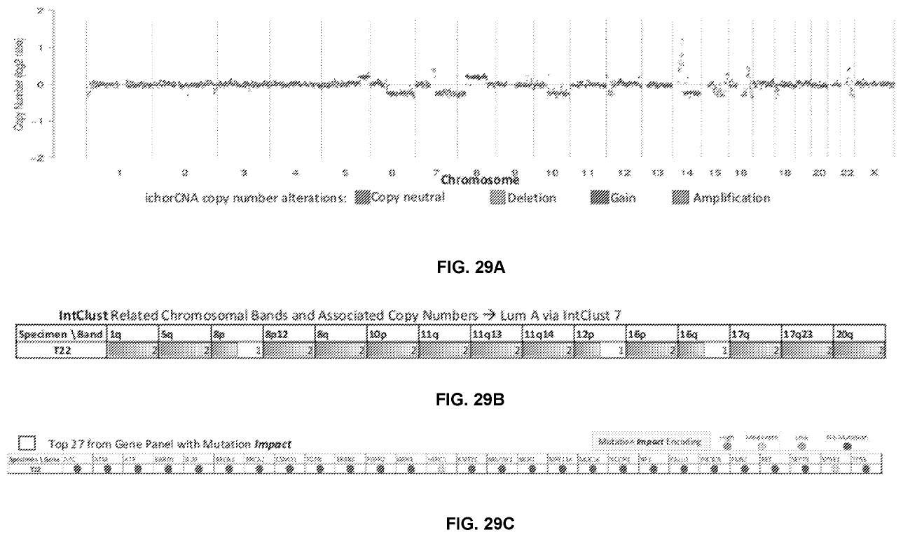

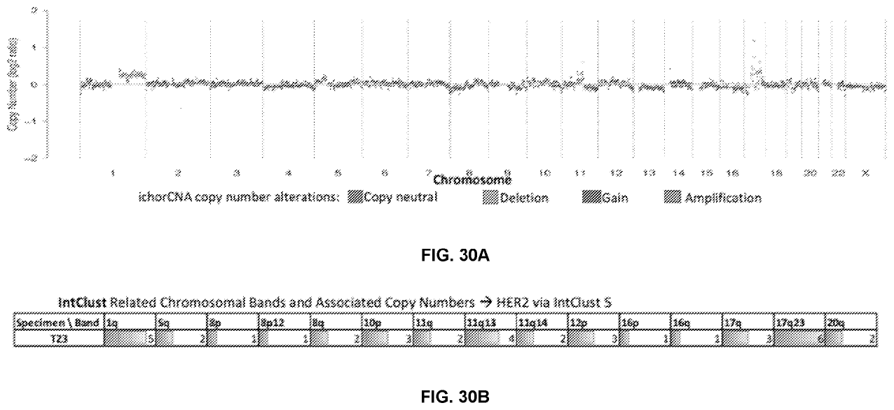

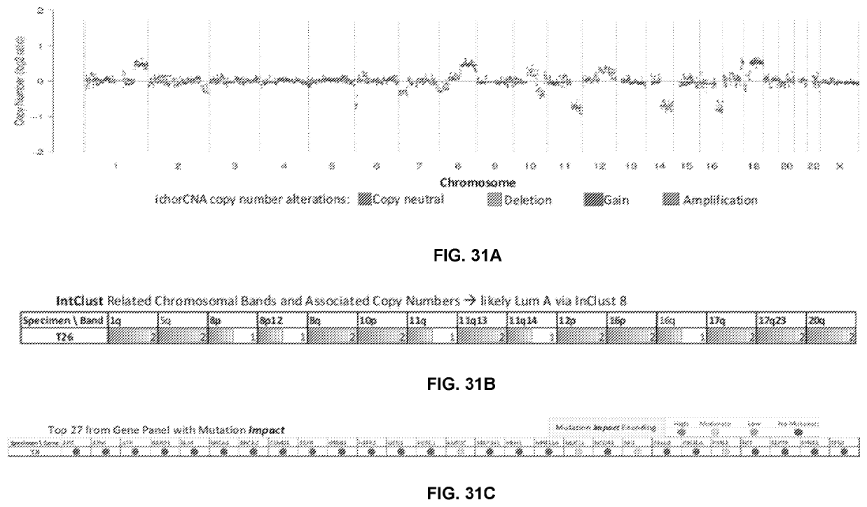

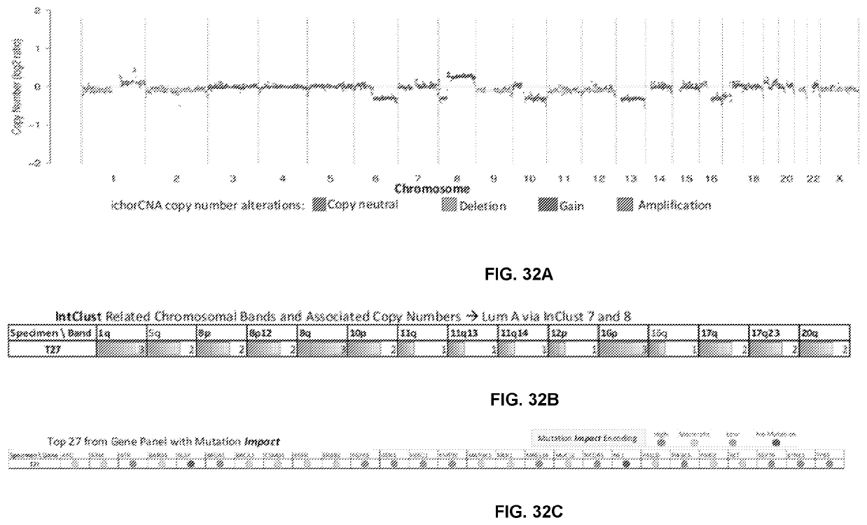

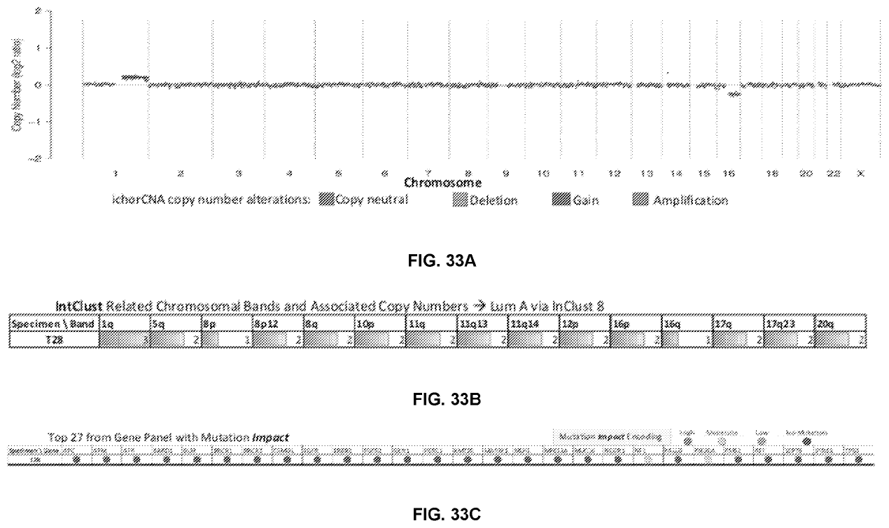

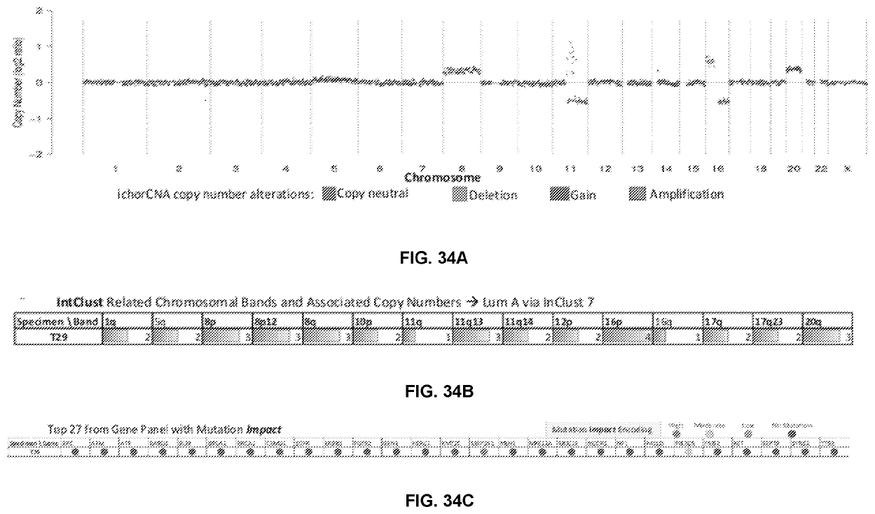

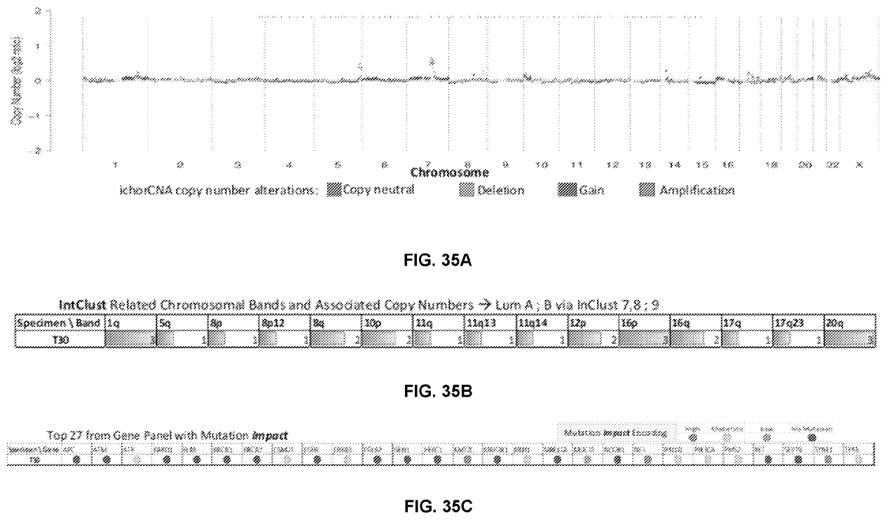

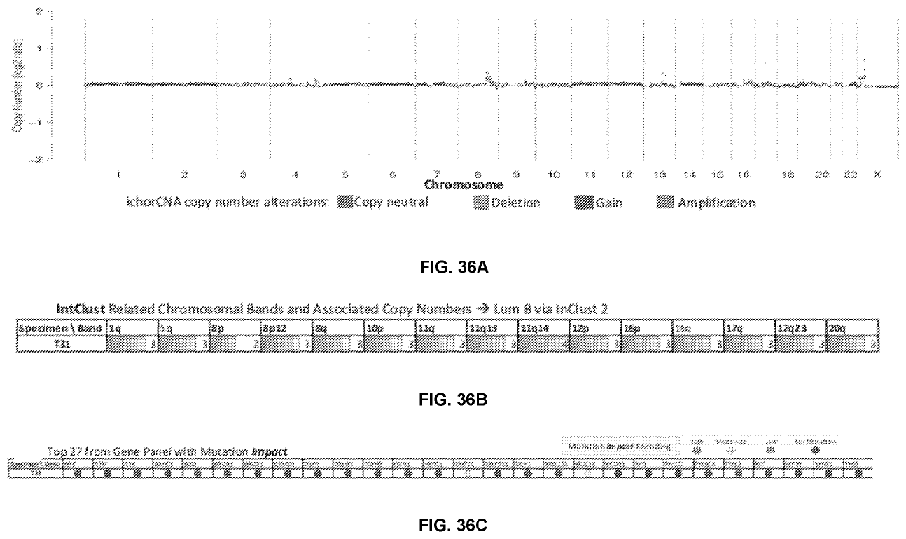

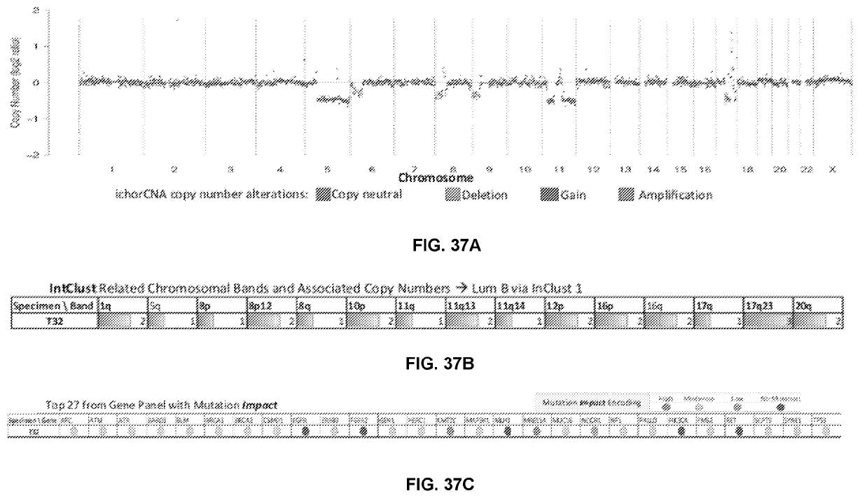

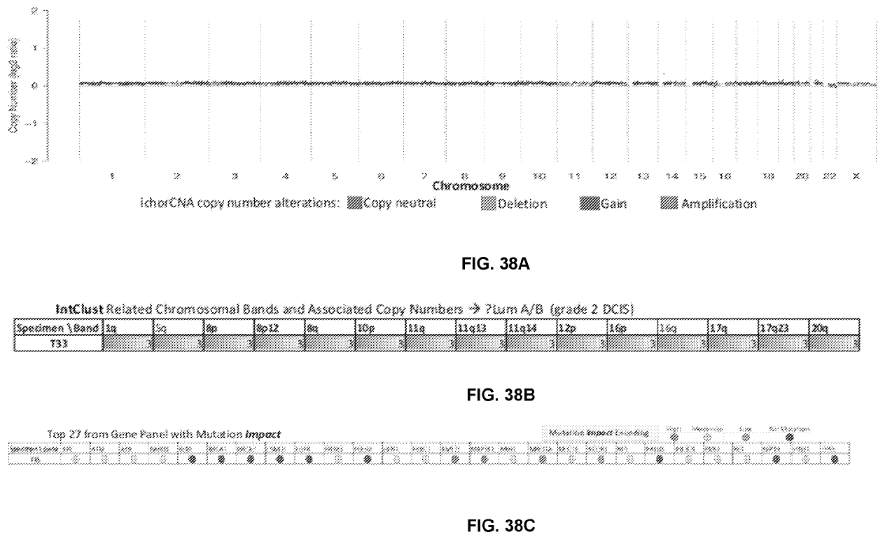

DNA NGS analyses performed included a targeted panel that was successful for library prep and NGS but displayed variable findings due to subclonal diversity, likely related to fixation artifacts, with potential for high false positive rates (FPR). To mitigate these findings, the approach was revised with a targeted 93 gene panel utilizing Unique Molecular Identifiers (UMIs) to reduce false positives, which was successful, and is illustrated in FIG. 7A, FIG. 8-38, Tables 25-53. Low-pass and in some instances ultra-low-pass WGS for Copy Number Variation (CNV) was also performed and was successful and are shown in FIG. 7A, FIG. 8-38, Tables 25-53, for specimens T1-T33.

In FIG. 7A, top row is shared by Ch Band which designates the IntClust chromosomal band and Specimen, which is the final specimen ID for this set of clinical and molecular results. Since Specimens T24 and T25 are cell line control materials, they are not included. Row IntClust Subtype displays the breast tumor molecular subtype specified by the IntClust classifier. The next row, Gene\ddPCR subtype introduces the top 27 genes from the UMI-based gene panel of 91 TCGA breast cancer driver genes, and color coded based on the mutational effect on protein function, with red as high effect, yellow as moderate, green as low, and black as no mutation. The ddPCR assay subtype is listed and color coded as Luminal A (LumA, blue), Luminal B (LumB, green), Triple Negative (TN, red), Her2 (pink), and if the specimen was predominantly DIS. Specimen T17 was not a tumor.

TABLE 25
Additional Details of Selected DNA Panel Mutations of Sample T6.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | Stop | High | 0.17 | 10675 | 4705 | COSV51275813 |
| | | | gained | | | | | |
| PIK3CA | c.3140A > G | p.His1047Arg | missense | Moderate | 0.29 | 795 | 403 | COSV55873195 |
| | | | variant | | | | | |
| NF1 | c.1400C > T | p.Thr467Ile | missense | Moderate | 0.12 | 2010 | 898 | COSV62197770 |
| | | | variant | | | | | |
| TP53 | c.818G > T | p.Arg273Leu | missense | Moderate | 0.03 | 1817 | 779 | COSV52664805 |
| | | | variant | | | | | |

TABLE 26
Additional Details of Selected DNA Panel Mutations of Sample T1.

| Gene | | | Classifi- | | Fre- | | MT | |
|---|---|---|---|---|---|---|---|---|
| Symbol | Transcript | Protein | cation | Impact | quency | Depth | Depth | COSMIC Id |
| KMT2C | c.2447dupA | p.Tyr816fs | Stop | High | 0.27 | 6595 | 3143 | COSV51275813 |
| | | | gained | | | | | |
| NF1 | c.1400C > T | p.Thr467Ile | Missense | Moderate | 0.13 | 1002 | 486 | COSV62197770 |
| | | | variant | | | | | |
| TP53 | c.376-2A > G | NULL | Splice | High | 0.08 | 1260 | 573 | COSV52666637 |
| | | | acceptor | | | | | |
| | | | variant | | | | | |
| RAD50 | c.2801delA | p.Asn934fs | Frameshift | High | 0.04 | 1304 | 623 | COSV54751030 |
| | | | variant | | | | | |
| BLM | c.1544delA | p.Asn515fs | Frameshift | High | 0.08 | 598 | 303 | COSV61921885 |
| | | | variant | | | | | |

TABLE 27
Additional Details of Selected DNA Panel Mutations of Sample T2.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | Stop | High | 0.28 | 8828 | 3790 | COSV51275813 |
| | | | gained | | | | | |
| ATM | c.5557G > A | p.Asp1853Asn | Missense | Moderate | 0.44 | 1471 | 629 | COSV53728020 |
| | | | variant | | | | | |
| NF1 | c.1400C > T | p.Thr467Ile | Missense | Moderate | 0.12 | 1430 | 619 | COSV62197770 |
| | | | variant | | | | | |
| TP53 | c.376-2A > G | | Splice | High | 0.08 | 1214 | 501 | COSV52666637 |
| | | | acceptor | | | | | |
| | | | variant | | | | | |

TABLE 28
Additional Details of Selected DNA Panel Mutations of Sample T3.

| | | | Classifi- | | Fre- | | MT | |
|---|---|---|---|---|---|---|---|---|
| Gene | Transcript | Protein | cation | Impact | quency | Depth | Depth | COSMIC Id |
| TP53 | c.376-2A > G | | Splice | High | 0.15 | 2082 | 871 | COSV52666637 |
| | | | acceptor | | | | | |
| | | | variant | | | | | |
| KMT2C | c.2447dupA | p.Tyr816fs | Stop | High | 0.29 | 12973 | 5846 | COSV51275813 |
| | | | gained | | | | | |
| ATM | c.5557G > A | p.Asp1853Asn | Missense | Moderate | 0.43 | 2520 | 1125 | COSV53728020 |
| | | | variant | | | | | |
| TP53 | c.215C > G | p.Pro72Arg | Missense | Moderate | 0.62 | 1925 | 856 | COSV52666208 |
| | | | variant | | | | | |
| PCGF2 | c.439C > T | p.Arg147* | Stop | High | 0.56 | 2219 | 1008 | |
| | | | gained | | | | | |

TABLE 29
Additional Details of Selected DNA Panel Mutations of Sample T4.

| | | | Classifi- | | Fre- | | MT | |
|---|---|---|---|---|---|---|---|---|
| Gene | Transcript | Protein | cation | Impact | quency | Depth | Depth | COSMIC Id |
| TP53 | c.376-2A > G | | Splice | High | 0.13 | 1855 | 708 | COSV52666637 |
| | | | acceptor | | | | | |
| | | | variant | | | | | |
| KMT2C | c.2447dupA | p.Tyr816fs | Stop | High | 0.30 | 11188 | 4485 | COSV51275813 |
| | | | gained | | | | | |
| MEN1 | c.1636A > G | p.Thr546Ala | Missense | Moderate | 0.99 | 5817 | 2293 | COSV53639974 |
| | | | variant | | | | | |
| PCGF2 | c.439C > T | p.Arg147* | Stop | High | 0.55 | 2040 | 854 | |
| | | | gained | | | | | |
| MUTYH | c.1276C > T | p.Arg426Cys | Missense | Moderate | 0.47 | 1885 | 804 | COSV58344995 |
| | | | variant | | | | | |

TABLE 30
Additional Details of Selected DNA Panel Mutations of Sample T5.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | Stop | High | 0.19 | 10349 | 4402 | COSV51275813 |
| | | | gained | | | | | |
| BRCA2 | c.9976A > T | p.Lys3326* | Stop | High | 0.51 | 784 | 374 | |
| | | | gained | | | | | |
| PIK3CA | c.3140A > G | p.His1047Arg | missense | Moderate | 0.24 | 766 | 368 | COSV55873195 |
| | | | variant | | | | | |
| NF1 | c.1400C > T | p.Thr467Ile | missense | Moderate | 0.11 | 1736 | 760 | COSV62197770 |
| | | | variant | | | | | |

TABLE 31
Additional Details of Selected DNA Panel Mutations of Sample T7.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.31 | 5788 | 2594 | COSV51275813 |
| | | | gained | | | | | |
| TRAF5 | c.434G > A | p.Arg145Gln | missense | Moderate | 0.35 | 176 | 85 | |
| | | | variant | | | | | |
| TP53 | c.159G > A | p.Trp53* | stop | High | 0.03 | 518 | 217 | COSV52751414 |
| | | | gained | | | | | |
| AR | c.1118G > A | p.Gly373Glu | missense | Moderate | 0.05 | 375 | 167 | |
| | | | variant | | | | | |
| BRAC2 | c.7150C > A | p.Gln2384Lys | missense | Moderate | 0.51 | 506 | 234 | |
| BRCA2 | c.9097delA | p.Thr3033fs | Frameshift | High | 0.04 | 600 | 286 | COSM1366491 |

TABLE 32
Additional Details of Selected DNA Panel Mutations of Sample T8.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.39 | 9365 | 3888 | COSV51275813 |
| | | | gained | | | | | |
| KMT2C | c.2959T > C | p.Tyr987His | missense | Moderate | 0.18 | 1345 | 514 | COSV51274668 |
| | | | variant | | | | | |
| BLM | c.1544delA | p.Asn515fs | frameshift | High | 0.04 | 803 | 330 | COSV61921885 |
| | | | variant | | | | | |
| TP53 | c.215C > G | p.Pro72Arg | missense | Moderate | 0.99 | 1155 | 442 | COSV52666208 |
| | | | variant | | | | | |

TABLE 33
Additional Details of Selected DNA Panel Mutations of Sample T9.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.24 | 10552 | 4459 | COSV51275813 |
| | | | gained | | | | | |
| RAD50 | c.2165delA | p.Lys722fs | frameshift | High | 0.03 | 1914 | 754 | COSV54748452 |
| | | | variant | | | | | |
| BLM | c.1544delA | p.Asn515fs | frameshift | High | 0.05 | 808 | 341 | COSV61921885 |
| | | | variant | | | | | |
| PIK3CA | c.1633G > A | p.Glu545Lys | missense | Moderate | 0.06 | 1377 | 582 | COSV55873239 |
| | | | variant | | | | | |
| MLH1 | c.655A > G | p.Ile219Val | missense | Moderate | 0.41 | 723 | 276 | COSV51613800 |
| | | | variant | | | | | |

TABLE 34
Additional Details of Selected DNA Panel Mutations of Sample T10.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.16 | 14609 | 4839 | COSV51275813 |
| | | | gained | | | | | |
| BRCA1 | c.2612C > T | p.Pro871Leu | missense | Moderate | 0.28 | 828 | 243 | COSV58784386 |
| | | | variant | | | | | |
| XRCC3 | c.722C > T | p.Thr241Met | missense | Moderate | 0.45 | 989 | 323 | COSV57974380 |
| | | | variant | | | | | |
| BRCA2 | c.1114A > C | p.Asn372His | missense | Moderate | 0.37 | 1375 | 476 | COSV66448817 |
| | | | variant | | | | | |
| TP53 | c.404G > A | p.Cys135Tyr | missense | Moderate | 0.36 | 882 | 285 | COSV52675774 |
| | | | variant | | | | | |

TABLE 35
Additional Details of Selected DNA Panel Mutations of Sample T11.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.17 | 12931 | 4916 | COSV51275813 |
| | | | gained | | | | | |
| BRCA1 | c.2612C > T | p.Pro871Leu | missense | Moderate | 0.34 | 759 | 270 | COSV58784386 |
| | | | variant | | | | | |
| BRCA1 | c.4462C > T | p.Gln1488* | Stop | High | 0.0302 | 1212 | 439 | |
| | | | gained | | | | | |
| XRCC3 | c.722C > T | p.Thr241Met | missense | Moderate | 0.42 | 990 | 383 | COSV57974380 |
| | | | variant | | | | | |
| BRCA2 | c.1114A > C | p.Asn372His | missense | Moderate | 0.38 | 1214 | 464 | COSV66448817 |
| | | | variant | | | | | |
| BRCA2 | c.7471C > T | p.Gln2491* | Stop | High | 0.0323 | 968 | 348 | |
| | | | gained | | | | | |

TABLE 36
Additional Details of Selected DNA Panel Mutations of Sample T12.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.17 | 11155 | 3729 | COSV51275813 |
| | | | gained | | | | | |
| BRCA1 | c.2612C > T | p.Pro871Leu | missense | Moderate | 0.35 | 383 | 129 | COSV58784386 |
| | | | variant | | | | | |
| BRCA1 | c.5017C > T | | protein | High | 0.04 | 827 | 255 | |
| | | | protein | | | | | |
| | | | contact | | | | | |
| BRCA2 | c.1114A > C | p.Asn372His | missense | Moderate | 0.42 | 1140 | 376 | COSV66448817 |
| | | | variant | | | | | |
| BRCA2 | c.8821C > T | p.Gln2941* | stop | High | 0.03 | 775 | 267 | |
| | | | gained | | | | | |

TABLE 37
Additional Details of Selected DNA Panel Mutations of Sample T13.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop | High | 0.20 | 12932 | 5594 | COSV51275813 |
| | | | gained | | | | | |
| TP53 | c.404G > A | p.Cys135Tyr | missense | Moderate | 0.39 | 963 | 413 | COSV52675774 |
| | | | variant | | | | | |
| XRCC3 | c.722C > T | p.Thr241Met | missense | Moderate | 0.44 | 1228 | 535 | COSV57974380 |
| | | | variant | | | | | |
| BRCA1 | c.2612C > T | p.Pro871Leu | missense | Moderate | 0.40 | 669 | 301 | COSV58784386 |
| | | | variant | | | | | |
| BRCA1 | c.4185 + | | splice_donor | High | 0.06 | 390 | 164 | |
| | 1G > A | | | | | | | |
| BRCA2 | c.1114A > C | p.Asn372His | missense | Moderate | 0.39 | 1488 | 644 | COSV66448817 |
| | | | variant | | | | | |
| BLM | c.1544delA | p.Asn515fs | frameshift | High | 0.06 | 732 | 324 | COSV61921885 |
| | | | variant | | | | | |
| RET | c.2136 + | | splice | High | 0.05 | 388 | 162 | COSV60689017 |
| | 1G > A | | donor | | | | | |
| | | | variant | | | | | |

TABLE 38
Additional Details of Selected DNA Panel Mutations of Sample T14.

| | | | Classifi- | | Fre- | | MT | |
|---|---|---|---|---|---|---|---|---|
| Gene | Transcript | Protein | cation | Impact | quency | Depth | Depth | COSMIC Id |
| PIK3CA | c.1624G > A | p.Glu542Lys | missense | Moderate | 0.45 | 19615 | 417 | COSV55873227 |
| | | | variant | | | | | |
| MAP3K1 | c.3982 + | | splice | High | 0.34 | 5360 | 278 | |
| | 2T > A | | donor | | | | | |
| | | | variant | | | | | |

TABLE 39
Additional Details of Selected DNA Panel Mutations of Sample T15.

| | | | Classifi- | | Fre- | | MT | |
|---|---|---|---|---|---|---|---|---|
| Gene | Transcript | Protein | cation | Impact | quency | Depth | Depth | COSMIC Id |
| PTEN | c.388C > G | | protein | High | 0.30 | 6826 | 447 | COSV64288384 |
| | | | protein | | | | | |
| | | | contact | | | | | |
| MAP3K1 | c.1817delC | p.Ser606fs | frameshift | High | 0.15 | 6915 | 829 | |
| | | | variant | | | | | |
| MAP3K1 | c.3989C > G | p.Ser1330Trp | missense | Moderate | 0.12 | 3085 | 288 | COSV68122713 |
| | | | variant | | | | | |

TABLE 40
Additional Details of Selected DNA Panel Mutations of Sample T16.

| | | | Classifi- | | Fre- | | MT | |
|---|---|---|---|---|---|---|---|---|
| Gene | Transcript | Protein | cation | Impact | quency | Depth | Depth | COSMIC Id |
| GATA3 | c.1201_{—} | p.Met401fs | frameshift | High | 0.16 | 4778 | 243 | COSV60521761 |
| | 1202delAT | | variant | | | | | |
| TP53 | c.215C > G | p.Pro72Arg | missense | Moderate | 0.65 | 10119 | 279 | COSV60521761 |
| | | | variant | | | | | |
| ITCH | c.569T > C | p.Ile190Thr | missense | Moderate | 0.04 | 7495 | 299 | |
| | | | variant | | | | | |
| MLH1 | c.2172G > T | p.Leu724Phe | missense | Moderate | 0.03 | 4277 | 162 | |
| | | | variant | | | | | |

TABLE 41
Additional Details of Selected DNA Panel Mutations of Sample T18.

| | | | | | | | MT | |
|---|---|---|---|---|---|---|---|---|
| Gene | Transcript | Protein | Classification | Impact | Frequency | Depth | Depth | COSMIC Id |
| GATA3 | c.925-3_925- | | splice region | High | 0.34 | 4139 | 329 | COSV60515023 |
| | 2delCA | | variant | | | | | |
| PIK3CA | c.3140A > G | p.His1047Arg | missense | Moderate | 0.29 | 7760 | 580 | COSV55873195 |
| | | | variant | | | | | |
| MUC16 | c.39790C > A | p.His13264Asn | missense | Moderate | 0.03 | 8500 | 936 | |
| | | | variant | | | | | |

TABLE 42
Additional Details of Selected DNA Panel Mutations of Sample T19.

| | | | Classifi- | | Fre- | | MT | |
|---|---|---|---|---|---|---|---|---|
| Gene | Transcript | Protein | cation | Impact | quency | Depth | Depth | COSMIC Id |
| SMARCA4 | c.3760G > T | p.Glu1254* | stop | High | 0.06 | 4027 | 73 | COSV60807407 |
| | | | gained | | | | | |
| APC | c.487C > T | p.Gln163* | stop | High | 0.06 | 2633 | 67 | |
| | | | gained | | | | | |
| FGFR1 | c.2200G > A | p.Gly734Ser | missense | Moderate | 0.04 | 3694 | 118 | COSV58337226 |
| | | | variant | | | | | |
| ATM | c.3174G > A | p.Trp1058* | missense | Moderate | 0.08 | 2726 | 68 | |
| | | | variant | | | | | |
| TP53 | c.377A > T | p.Tyr126Cys | missense | Moderate | 0.03 | 3932 | 110 | COSV52689293 |
| | | | variant | | | | | |
| NF1 | c.1400C > T | p.Thr467Ile | missense | Moderate | 0.19 | 2316 | 75 | COSV62197770 |
| | | | variant | | | | | |

TABLE 43
Additional Details of Selected DNA Panel Mutations of Sample T20.

| | | | | | | | MT | |
|---|---|---|---|---|---|---|---|---|
| Gene | Transcript | Protein | Classification | Impact | Frequency | Depth | Depth | COSMIC Id |
| GATA3 | c.925-3_925- | | splice | High | 0.30 | 3738 | 113 | COSV60515023 |
| | 2delCA | | region | | | | | |
| | | | variant | | | | | |
| TP53 | c.215C > G | p.Pro72Arg | missense | Moderate | 0.78 | 6733 | 201 | COSV52666208 |
| | | | variant | | | | | |
| PALLD | c.1260 + | | splice | High | 0.03 | 4205 | 127 | COSV54983265 |
| | 1G > T | | donor | | | | | |
| | | | variant | | | | | |
| MAP3K1 | c.2845_{—} | p.Thr949del | inframe | Moderate | 0.93 | 13531 | 730 | |
| | 2847delACA | | deletion | | | | | |
| MAP3K1 | c.3515A > C | p.Asn1172Thr | missense | Moderate | 0.40 | 5788 | 285 | |
| | | | variant | | | | | |

TABLE 44
Additional Details of Selected DNA Panel Mutations of Sample T21.

| | | | | | | | MT | |
|---|---|---|---|---|---|---|---|---|
| Gene | Transcript | Protein | Classification | Impact | Frequency | Depth | Depth | COMIC Id |
| TP53 | c.617T > A | p.Leu206* | stop gained | High | 0.13 | 6325 | 997 | COSV52853973 |
| RET | c.2071G > A | p.Gly691Ser | missense | Moderate | 0.57 | 12770 | 1535 | COSV60687096 |
| | | | variant | | | | | |
| AR | c.2216A > T | p.Gln739Leu | missense | Moderate | 0.06 | 7824 | 1203 | |
| | | | variant | | | | | |
| HERC1 | c.5044C > G | p.Leu1682Val | missense | Moderate | 0.60 | 11349 | 1901 | |
| | | | variant | | | | | |

TABLE 45
Additional Details of Selected DNA Panel Mutations of Sample T22.

| | | | Classifi- | | Fre- | | MT | |
|---|---|---|---|---|---|---|---|---|
| Gene | Transcript | Protein | cation | Impact | quency | Depth | Depth | COMIC Id |
| GATA3 | c.987_{—} | p.Arg331fs | frameshift | High | 0.09 | 5213 | 594 | COSV60522567 |
| | 990dupGAGG | | variant | | | | | |
| HERC1 | c.6658A > G | p.Ile2220Val | missense | Moderate | 0.61 | 5334 | 723 | COSV71246773 |
| | | | variant | | | | | |
| SYNE1 | c.13786T > A | p.Ser4596Thr | missense | Moderate | 0.65 | 4716 | 491 | COSV54944634 |
| | | | variant | | | | | |
| ACVR1B | c.1454delG | p.Arg485fs | frameshift | High | 0.14 | 6104 | 642 | |
| | | | variant | | | | | |

TABLE 46
Additional Details of Selected DNA Panel Mutations of Sample T26.

| | | | Classifi- | | Fre- | | MT | |
|---|---|---|---|---|---|---|---|---|
| Gene | Transcript | Protein | cation | Impact | quency | Depth | Depth | COMIC Id |
| NF1 | c.1400C > T | p.Thr467Ile | missense | Moderate | 0.08 | 1857 | 324 | COSV62197770 |
| | | | variant | | | | | |
| KMT2C | c.851G > A | p.Arg284Gln | missense | Moderate | 0.05 | 1762 | 300 | COSV51275388 |
| | | | variant | | | | | |
| AR | c.234_{—} | p.Gln79_{—} | disruptive | Moderate | 0.07 | 2404 | 683 | COSV65952724 |
| | 239delGCAGCA | Gln80del | inframe | | | | | |
| | | | deletion | | | | | |
| GATA3 | c.244A > T | p.Ser82Cys | missense | Moderate | 0.33 | 3988 | 495 | |
| | | | variant | | | | | |

TABLE 47
Additional Details of Selected DNA Panel Mutations of Sample T27.

| | | | Classifi- | | Fre- | | MT | |
|---|---|---|---|---|---|---|---|---|
| Gene | Transcript | Protein | cation | Impact | quency | Depth | Depth | COSMIC Id |
| NF1 | c.2617C > T | p.Arg873Cys | missense | Moderate | 0.16 | 2187 | 334 | COSV62191947 |
| | | | variant | | | | | |
| PIK3CA | c.3140A > G | p.His1047Arg | missense | Moderate | 0.11 | 1845 | 241 | COSV55873195 |
| | | | variant | | | | | |
| CBFB | c.282 + | | splice | High | 0.18 | 2624 | 385 | |
| | 2T > C | | donor | | | | | |
| | | | variant | | | | | |

TABLE 48
Additional Details of Selected DNA Panel Mutations of Sample T28.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.3358G > T | p.Glu1120* | stop gained | High | 0.15 | 1922 | 56 | COSV51487946 |
| WEE1 | c.1102G > A | | protein | High | 0.11 | 1182 | 54 | COSV55196867 |
| | | | protein | | | | | |
| | | | contact | | | | | |
| GATA3 | c.473C > T | p.Pro158Leu | missense | Moderate | 0.10 | 1303 | 91 | COSV60515912 |
| | | | variant | | | | | |
| CTNNB1 | c.830G > A | p.Gly277Asp | missense | Moderate | 0.15 | 786 | 41 | COSV62697081 |
| | | | variant | | | | | |
| SYNE1 | c.3925C > T | p.Arg1309* | stop gained | High | 0.06 | 2416 | 73 | COSV55017647 |
| PTEN | c.451G > A | p.Ala151Thr | missense | Moderate | 0.04 | 2046 | 116 | COSV64288600 |
| | | | variant | | | | | |
| FGFR1 | c.659G > A | p.Arg220His | missense | Moderate | 0.06 | 1976 | 85 | COSV58331880 |
| | | | variant | | | | | |
| ERBB2 | c.2350C > T | p.Arg784Cys | missense | Moderate | 0.07 | 1797 | 71 | COSV54065970 |
| | | | variant | | | | | |

TABLE 49
Additional Details of Selected DNA Panel Mutations of Sample T29.

| | | | Classifi- | | Fre- | | MT | |
|---|---|---|---|---|---|---|---|---|
| Gene | Transcript | Protein | cation | Impact | quency | Depth | Depth | COSMIC Id |
| PIK3CA | c.1633G > A | p.Glu545Lys | missense | Moderate | 0.31 | 1908 | 426 | COSV55873239 |
| | | | variant | | | | | |
| MAP3K1 | c.816_{—} | p.Arg273fs | frameshift | High | 0.28 | 2833 | 665 | |
| | 817delAA | | variant | | | | | |
| SMAD4 | c.787 + | | splice | Low | 0.28 | 2890 | 676 | |
| | 5G > A | | region | | | | | |
| | | | variant | | | | | |

TABLE 50
Additional Details of Selected DNA Panel Mutations of Sample T30.

| | | | Classifi- | | Fre- | | MT | |
|---|---|---|---|---|---|---|---|---|
| Gene | Transcript | Protein | cation | Impact | quency | Depth | Depth | COSMIC Id |
| PIK3CA | c.3140A > G | p.His1047Arg | missense | Moderate | 0.15 | 1774 | 222 | COSV55873195 |
| | | | variant | | | | | |
| KMT2C | c.8390delA | p.Lys2797fs | frameshift | High | 0.04 | 2509 | 400 | COSV51277546 |
| | | | variant | | | | | |
| ATR | c.7312G > A | p.Glu2438Lys | missense | Moderate | 0.03 | 1583 | 204 | COSV63388147 |
| | | | variant | | | | | |
| PALLD | c.83C > T | p.Pro28Leu | missense | Moderate | 0.04 | 2115 | 419 | COSV54996794 |
| | | | variant | | | | | |
| TP53 | c.389T > A | p.Leu130His | missense | Moderate | 0.07 | 2424 | 332 | COSV52681833 |
| | | | variant | | | | | |

TABLE 51
Additional Details of Selected DNA Panel Mutations of Sample T31.

| | | | | | | | MT | |
|---|---|---|---|---|---|---|---|---|
| Gene | Transcript | Protein | Classification | Impact | Frequency | Depth | Depth | COSMIC Id |
| KMT2C | c.2185A > G | p.Asn729Asp | missense | Moderate | 0.05 | 2120 | 441 | COSV51279322 |
| | | | variant | | | | | |
| MUC16 | c.39020_{—} | p.Val13007Gly | missense | Moderate | 0.09 | 3269 | 692 | |
| | 39021delTTinsGC | | variant | | | | | |

TABLE 52
Additional Details of Selected DNA Panel Mutations of Sample T32.

| | | | | | | | MT | |
|---|---|---|---|---|---|---|---|---|
| Gene | Transcript | Protein | Classification | Impact | Frequency | Depth | Depth | COSMIC Id |
| KMT2C | c.2447dupA | p.Tyr816fs | stop gained | High | 0.27 | 14457 | 2375 | COSV51275813 |
| SYNE1 | c.13786T > A | p.Ser4596Thr | missense | Moderate | 0.43 | 1620 | 240 | COSV54944634 |
| | | | variant | | | | | |
| BRCA1 | c.2612C > T | p.Pro871Leu | missense | Moderate | 0.30 | 1346 | 217 | COSV58784386 |
| | | | variant | | | | | |

TABLE 53
Additional Details of Selected DNA Panel Mutations of Sample T33.

| | | | Classifi- | | Fre- | | MT | |
|---|---|---|---|---|---|---|---|---|
| Gene | Transcript | Protein | cation | Impact | quency | Depth | Depth | COSMIC Id |
| KMT2C | c.2447dupA | p.Tyr816fs | stop gained | High | 0.20 | 12932 | 5594 | COSV51275813 |
| TP53 | c.404G > A | p.Cys135Tyr | missense | Moderate | 0.39 | 963 | 413 | COSV52675774 |
| | | | variant | | | | | |
| XRCC3 | c.722C > T | p.Thr241Met | missense | Moderate | 0.44 | 1228 | 535 | COSV57974380 |
| | | | variant | | | | | |
| BRCA1 | c.2612C > T | p.Pro871Leu | missense | Moderate | 0.40 | 669 | 301 | COSV58784386 |
| | | | variant | | | | | |
| BRCA1 | c.4185 + | | splice_donor | High | 0.06 | 390 | 164 | |
| | 1G > A | | | | | | | |
| BRCA2 | c.1114A > C | p.Asn372His | missense | Moderate | 0.39 | 1488 | 644 | COSV66448817 |
| | | | variant | | | | | |
| BLM | c.1544delA | p.Asn515fs | frameshift | High | 0.06 | 732 | 324 | COSV61921885 |
| | | | variant | | | | | |
| RET | c.2136 + | | splice donor | High | 0.05 | 388 | 162 | COSV60689017 |
| | 1G > A | | variant | | | | | |

### Example 5: Clinical and Genomic Features

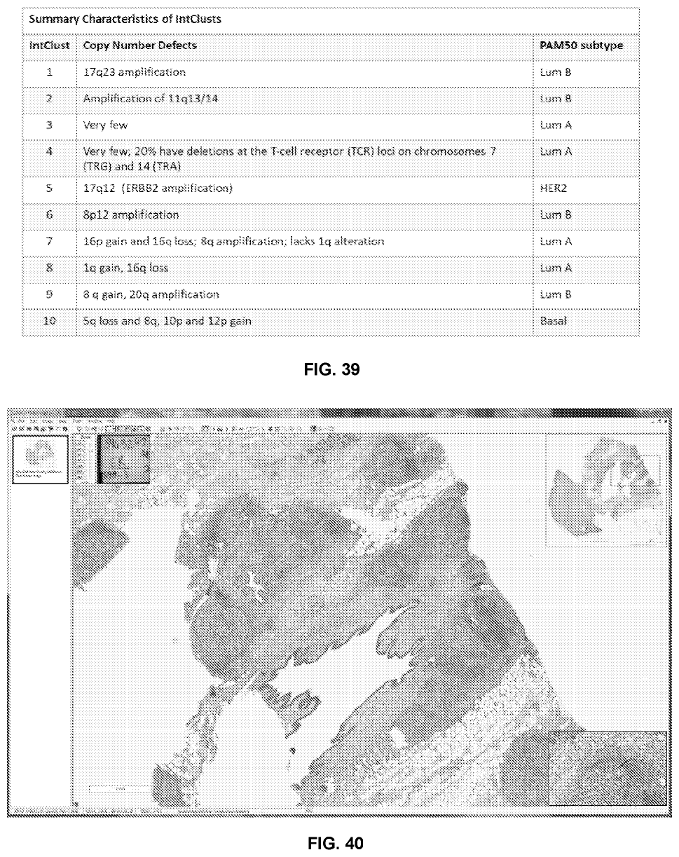

The clinical and genomic features of all breast cancer tumor specimens are illustrated in FIG. 7. The chromosomal bands (Ch Band) used in the IntClust algorithm along with the Specimen (T1-T33) identifier are listed and correspond to the entire first column and row, respectively. Specimens T24, 25 are RNA controls and are not included. The next row reports the IntClust Subtype, is listed and directly maps/correlates to the PAM50 subtype, and FIG. 39 explains the copy number mapping criteria to PAM50 subtypes. As background, the tissue specimens for T1-T13 used in the IntClust subtype analysis (ie, low-pass or ultra-low pass WGS) were from different tumor blocks or a significantly different block positions versus what was used in the ddPCR subtype assay. This was due to the FFPE block being consumed over time. Thus, solid tissue/tumor heterogeneity is likely and may help explain the tumor subtype differences reported by IntClust and ddPCR, especially for specimens T1 and T4. The row heading “Gene ddPCR Subtype” is next. The ddPCR molecular subtype is listed as determined by the custom assay, and as mentioned previously, for eight specimens (T26-T33) the RNA was consumed prior to running this assay. The subsequent aspect of this figure reports a matrix of findings concerning the top 27 genes from all specimens (T1-T33) along with color coding of the mutation according to the impact effect on protein function. The histopathology (HistoPath) is reported in following row, as determined by a pathologist (UAMS) and is color coded. The Tumor Grade is also reported as determined by a UAMS pathologist.

### Example 6: Analysis of DNA Specimens

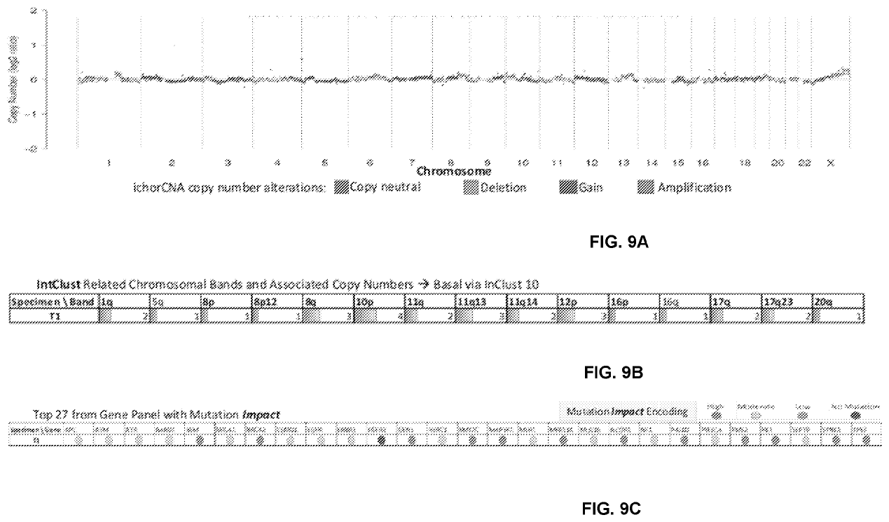

Each DNA tumor specimen underwent four analyses and an example involving specimen T6 is illustrated in FIG. 8. Results for all specimens (T1-T33) are shown in FIG. 9-38. FIG. 8A details the results of ichorCNA copy number alternations, with the x-axis listing chromosomes, the y-axis as log 2 ratio of copy number of the tumor specimen with a matched normal LN (if available) or ichor pooled normal (if unavailable), along with color coding of the copy number alterations. IntClust related chromosomal bands and associated copy number classification are shown in FIG. 8B, and specimen T6 was classified as Luminal A via the criteria from IntClust 8 (FIG. 39).

The top 27 gene targets from the gene panel along with color coding according to the mutation impact upon protein function is shown in FIG. 8C is presented next. Mutation impact encoding includes High impact on protein function (red), Moderate (yellow), Low (green), or No Mutation (black). Finally, additional details of selected DNA panel mutations are shown in Table 25 and include: Gene symbol, Transcript and Protein with mutational annotations, Classification of the mutation type along with the Impact on protein function, allelic Frequency, read Depth, and molecular tag (UMI) depth (MT Depth). If available, the COSMIC Id, was reported and further indicated clinical relevance of the mutation.

### Example 7: Further Correlating Specimens with Molecular Results and Pathology

A correlated summary of the molecular results with pathology for study specimens is listed in Table 54. Column-based descriptions of this table begin with, Initial Specimen ID, which is included since the specimens came from two different cohorts, S8897 and UAMS, and include specimens from breast tumors, normal LNs and cell lines. As previously stated, specimens T1-T13 are tumor block specimens derived from six unique patients and the Initial Specimen UDs are colored to show origin/membership among the six patients. Final Specimen ID serves to harmonize specimen naming for tumor specimens (T1-T33) and LN1-LN13, which are LN specimens used in ddPCR experiments and assay development given their adequate tissue availability. All LN specimens were from the S8897 cohort. Regarding tumor specimens, T1-T13, were from the UAMS specimen cohort, T24 and T25 were cancer cell lines used for ddPCR controls, and T14-T22 and T26-T33 were from the S8897 cohort.

TABLE 54
Summary of the molecular results with pathology for study specimens

| Initial | Final | | | | | | | |
|---|---|---|---|---|---|---|---|---|
| Speci- | Speci- | | | | PAM50 | scmod2 | | |
| men | men | IHC | IHC | IHC | Subtype | subtype | ddPCR | IntClust |
| ID | ID | ER | PR | Her2 | (RNAseq) | (RNAseq) | Subtype | Subtype |
| S98_3 | T1 | N | N | N | Basal | ER−/ | LumB | Basal |
| | | | | | | HER2− | | |
| S98_4 | T2 | N | N | N | Basal | ER−/ | TN | Basal |
| | | | | | | HER2− | | |
| S98_5 | T3 | N | N | N | Basal | ER−/ | TN | Basal |
| | | | | | | HER2− | | |
| S98_6 | T4 | N | N | N | Normal | ER−/ | LumB | Basal |
| | | | | | | HER2− | | |
| S98_8 | T5 | P | P | N | LumA | ER+/ | LumA | LumA |
| | | | | | | HER2−, | | |
| | | | | | | Low Prolif | | |
| S98_9 | T6 | P | P | N | LumA | ER+/ | LumA | LumA |
| | | | | | | HER2−, | | |
| | | | | | | Low Prolif | | |
| S98_11 | T7 | P | N | N | LumB | ER+/ | LumB | LumA/B |
| | | | | | | HER2−, | | |
| | | | | | | High Prolif | | |
| S98_12 | T8 | P | N | N | LumA | ER+/ | LumA | LumA/B |
| | | | | | | HER2−, | | |
| | | | | | | Low Prolif | | |
| S99_7 | T9 | N | N | P | LumA | HER2+ | HER2 | LumA/ |
| | | | | | | | | HER2 |
| S99_16 | T10 | N | N | N | Basal | ER−/ | TN | Basal |
| | | | | | | HER2− | | |
| S99_17 | T11 | N | N | N | Basal | ER−/ | TN | Basal |
| | | | | | | HER2− | | |
| S99_18 | T12 | N | N | N | Basal | ER−/ | TN | Basal |
| | | | | | | HER2− | | |
| S99_19 | T13 | N | N | N | Basal | ER−/ | TN | Basal |
| | | | | | | HER2− | | |
| Tu2 | T14 | * | * | * | * | * | LumA | LumA |
| Tu3 | T15 | * | * | * | * | * | LumA | LumA |
| Tu11 | T16 | * | * | * | * | * | LumA | LumA/B |
| Tu12 | T17 | * | * | * | * | * | NA | NA |
| Tu13 | T18 | * | * | * | * | * | LumA | LumA |
| Tu15 | T19 | * | * | * | * | * | LumB | LumA |
| Tu16 | T20 | * | * | * | * | * | LumA | LumA |
| Tu18 | T21 | * | * | * | * | * | TN | Basal/ |
| | | | | | | | | LumB |
| Tu19 | T22 | * | * | * | * | * | LumA | LumA |
| Tu20 | T23 | * | * | * | * | * | HER2 | HER2 |
| MCF7 | T24 | * | * | * | * | * | LumB | * |
| MB-231 | T25 | * | * | * | * | * | TN, weak | * |
| | | | | | | | HER2 | |
| Tu1 | T26 | * | * | * | * | * | RNA | LumA |
| | | | | | | | not avail | |
| Tu4 | T27 | * | * | * | * | * | RNA | LumA |
| | | | | | | | not avail | |
| Tu5 | T28 | * | * | * | * | * | RNA | LumA |
| | | | | | | | not avail | |
| Tu6 | T29 | * | * | * | * | * | RNA | LumA |
| | | | | | | | not avail | |
| Tu7 | T30 | * | * | * | * | * | RNA | LumA/B |
| | | | | | | | not avail | |
| Tu8 | T31 | * | * | * | * | * | RNA | LumB |
| | | | | | | | not avail | |
| Tu10 | T32 | * | * | * | * | * | RNA | LumB |
| | | | | | | | not avail | |
| Tu14 | T33 | * | * | * | * | * | RNA | LumA |
| | | | | | | | not avail | |
| LN1 | LN1 | * | * | * | * | * | NA (LN) | * |
| LN2 | LN2 | * | * | * | * | * | NA (LN) | * |
| LN3 | LN3 | * | * | * | * | * | NA (LN) | * |
| LN4 | LN4 | * | * | * | * | * | NA (LN) | * |
| LN5 | LN5 | * | * | * | * | * | NA (LN) | * |
| LN6 | LN6 | * | * | * | * | * | NA (LN) | * |
| LN11 | LN7 | * | * | * | * | * | NA (LN) | * |
| LN13 | LN8 | * | * | * | * | * | NA (LN) | * |
| LN14 | LN9 | * | * | * | * | * | NA (LN) | * |
| LN16 | LN10 | * | * | * | * | * | NA (LN) | * |
| LN17 | LN11 | * | * | * | * | * | NA (LN) | * |
| LN18 | LN12 | * | * | * | * | * | NA (LN) | * |
| LN19 | LN13 | * | * | * | * | * | NA (LN) | * |

| | Pathologist readings | | | | | |
|---|---|---|---|---|---|---|
| Initial | | | | % | | |
| Speci- | | Inva- | | Normal | | |
| men | | sive, | % | Tumor | | |
| ID | Notes | Insitu | Tumor | grade | | Comments |
| S98_3 | no matched | Inv | 30 | 70 | 3 | invasive ductal |
| | NL tissue | | | | | carcinoma with |
| | | | | | | extensive |
| | | | | | | inflamation |
| S98_4 | no matched | Inv | 40 | 60 | 3 | invasive ductal |
| | NL tissue | | | | | carcinoma with |
| | | | | | | extensive |
| | | | | | | inflamation |
| S98_5 | no matched | Inv | 40 | 60 | 3 | invasive ductal |
| | NL tissue | | | | | carcinoma with |
| | | | | | | extensive |
| | | | | | | inflamation |
| S98_6 | no matched | Inv | 40 | 60 | 3 | invasive ductal |
| | NL tissue | | | | | carcinoma with |
| | | | | | | extensive |
| | | | | | | inflamation |
| S98_8 | no matched | Inv | 70 | 30 | 1 | lobular |
| | NL tissue | | | | | carcinoma |
| S98_9 | no matched | Inv | 60 | 40 | 1 | lobular |
| | NL tissue | | | | | carcinoma |
| S98_11 | no matched | Inv | 50 | 50 | 2 | metastatic |
| | NL tissue | | | | | carcinoma in LN |
| S98_12 | no matched | Inv | 50 | 50 | 2 | invasive lobular |
| | NL tissue | | | | | carcinoma with |
| | | | | | | necrosis & some |
| | | | | | | crush artifact |
| S99_7 | no matched | DCIS | 30 | 70 | 2 | DCIS with |
| | NL tissue | | | | | mucinous changes |
| S99_16 | no matched | Inv | 60 | 40 | 3 | invasive ductal |
| | NL tissue | | | | | carcinoma with |
| | | | | | | solid pattern |
| S99_17 | no matched | Inv | 80 | 20 | 3 | invasive ductal |
| | NL tissue | | | | | carcinoma with |
| | | | | | | solid pattern |
| S99_18 | no matched | Inv | 50 | 50 | 3 | invasive ductal |
| | NL tissue | | | | | carcinoma with |
| | | | | | | solid pattern |
| | | | | | | and focal clear |
| | | | | | | cell features |
| S99_19 | no matched | Inv | 70 | 30 | 3 | invasive ductal |
| | NL tissue | | | | | carcinoma with |
| | | | | | | solid pattern |
| | | | | | | and focal clear |
| | | | | | | cell features |
| Tu2 | LN2, | Inv | 50 | 50 | 2 | |
| | matched NL | | | | | |
| Tu3 | LN3, | Inv | 95 | 5 | 2 | Comedo in rare |
| | matched NL | | | | | DCIS |
| Tu11 | LN11, | Inv | 15-20 | 80-85 | 2 | Tumor at edge of |
| | matched NL | | | | | tissue, fatty breast |
| Tu12 | Not a | NA | 0 | 100 | NA | Possible rare ADH |
| | tumor | | | | | and/or ALH |
| Tu13 | LN13, | DCIS | 80 (4:1 = | 20 | 1 | tumor necrosis |
| | matched NL | & Inv | DCIS:Inv) | | | in DCIS, comedo |
| Tu15 | LN15, | Inv | 20 | 80 | 2 | 10-20% immune |
| | matched NL | | | | | cells |
| Tu16 | LN16, | DCIS | 25 (4:1 = | 75 | 1 | Focal |
| | matched NL | & Inv | DCIS:Inv) | | | calcifications |
| | | | | | | in DCIS |
| Tu18 | LN18, | Inv | <5 | >95 | 2 | Scant tumor at |
| | matched NL | | | | | the edge of |
| | | | | | | the tissue |
| Tu19 | LN19, | Inv | 100 | 0 | 1 | |
| | matched NL | | | | | |
| Tu20 | LN20, | DCIS | 50 (4:1 = | 50 | 2 | Tumor in 1 out of |
| | matched NL | & Inv | DCIS:Inv) | | | 3 pieces |
| | | | | | | (biggest piece) |
| MCF7 | control for | NA | 100 | 0 | NA | MCF-7 cell line |
| | ddPCR | | | | | |
| MB-231 | control for | NA | 100 | 0 | NA | MDA-MB-231 |
| | ddPCR | | | | | cell line |
| Tu1 | RNA | Inv | 90 | 10 | 1 | |
| | consumed | | | | | |
| | in early test | | | | | |
| Tu4 | RNA | Inv | 100 | 0 | 2 | microcalcificatins |
| | consumed | | | | | in tumor |
| | in early test | | | | | |
| Tu5 | RNA | Inv | 60 | 40 | 1 | |
| | consumed | | | | | |
| | in early test | | | | | |
| Tu6 | RNA | Inv & | 95 | 5 | 2 | |
| | consumed | focal | | | | |
| | in early test | DCIS | | | | |
| Tu7 | RNA | Inv & | 40 (4:1 = | 60 | 3 | tumor necrosis |
| | consumed | DCIS | Inv:DCIS) | | | in DCIS |
| | in early test | | | | | |
| Tu8 | RNA | Inv | 30 | 70 | 3 | 70-80% immune |
| | consumed | | | | | cells |
| | in early test | | | | | |
| Tu10 | No matched | Inv & | 100 (9:1, | 0 | 2 | |
| | LN; RNA | DCIS | Inv:DCIS) | | | |
| | consumed | | | | | |
| Tu14 | RNA | DCIS | 15 | 85 | 2 | |
| | consumed | | | | | |
| | in early test | | | | | |
| LN1 | tissue group | NA | 0 | NA | NA | NL lymph |
| | for ddPCR | | | | | node (LN) |
| LN2 | tissue group | NA | 0 | NA | NA | NL LN |
| | for ddPCR | | | | | |
| LN3 | tissue group | NA | 0 | NA | NA | NL LN |
| | for ddPCR | | | | | |
| LN4 | tissue group | NA | 0 | NA | NA | NL LN |
| | for ddPCR | | | | | |
| LN5 | tissue group | NA | 0 | NA | NA | NL LN |
| | for ddPCR | | | | | |
| LN6 | tissue group | NA | 0 | NA | NA | NL LN |
| | for ddPCR | | | | | |
| LN11 | tissue group | NA | 0 | NA | NA | NL LN |
| | for ddPCR | | | | | |
| LN13 | tissue group | NA | 0 | NA | NA | NL LN |
| | for ddPCR | | | | | |
| LN14 | tissue group | NA | 0 | NA | NA | NL LN |
| | for ddPCR | | | | | |
| LN16 | tissue group | NA | 0 | NA | NA | NL LN |
| | for ddPCR | | | | | |
| LN17 | tissue group | NA | 0 | NA | NA | NL LN |
| | for ddPCR | | | | | |
| LN18 | tissue group | NA | 0 | NA | NA | NL LN |
| | for ddPCR | | | | | |
| LN19 | tissue group | NA | 0 | NA | NA | NL LN |
| | for ddPCR | | | | | |

* denotes the assay was not run.
The following specimens (tumor blocks) were from the same patients: T1-4; T5-6; T10-13
Abbreviatons: Inv, Invasive; NA, Not Applicable; N, Negative; NL, Normal; P, Positive

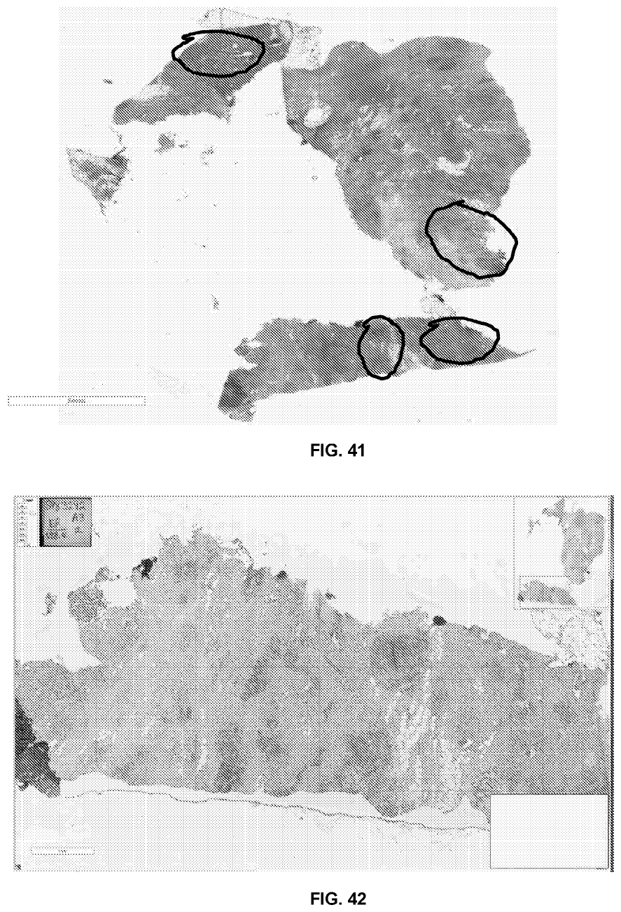

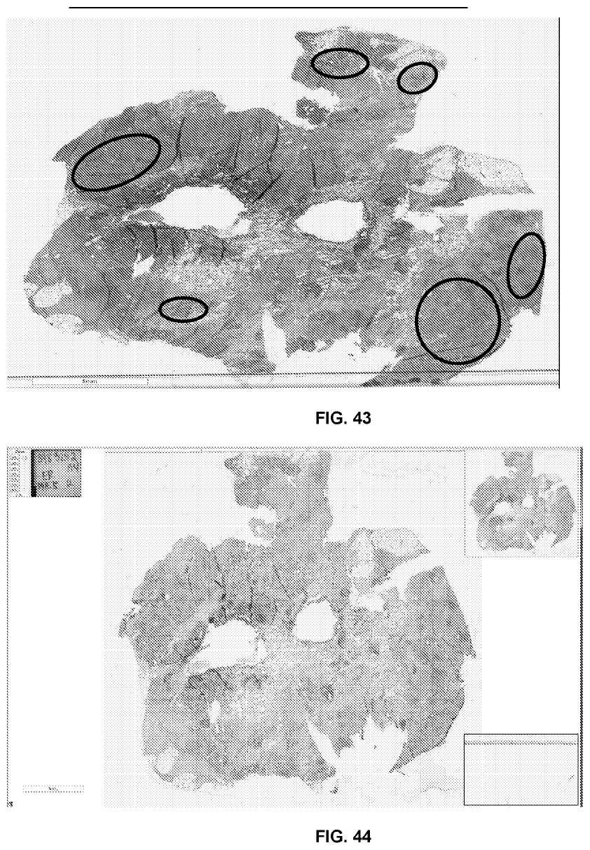

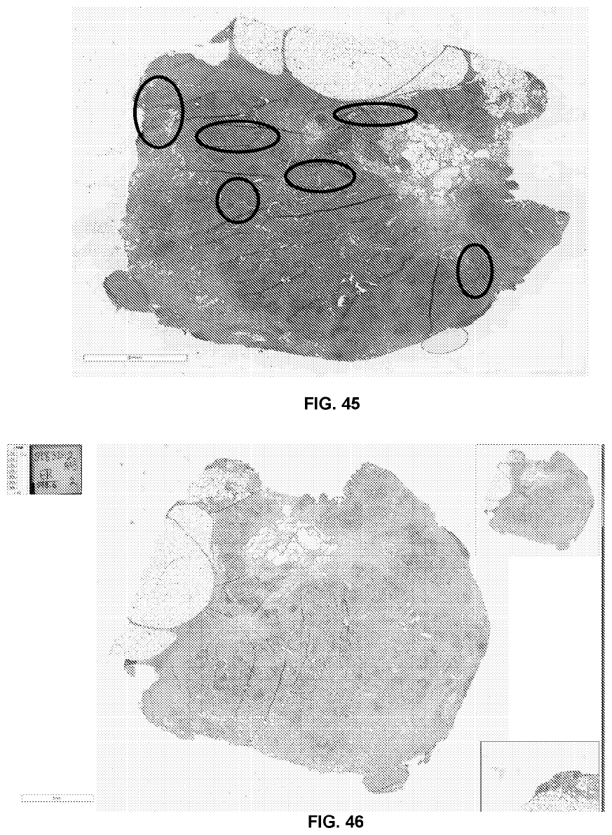

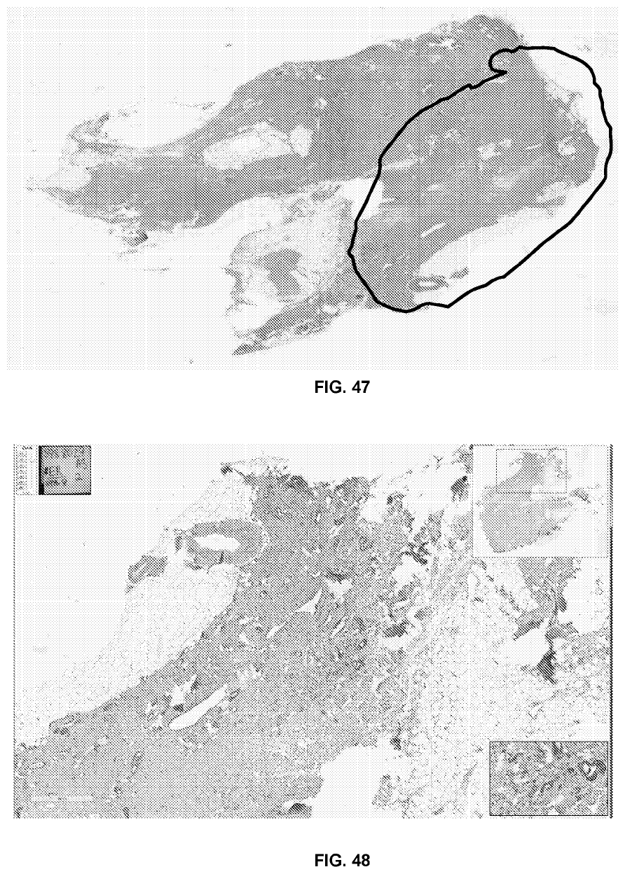

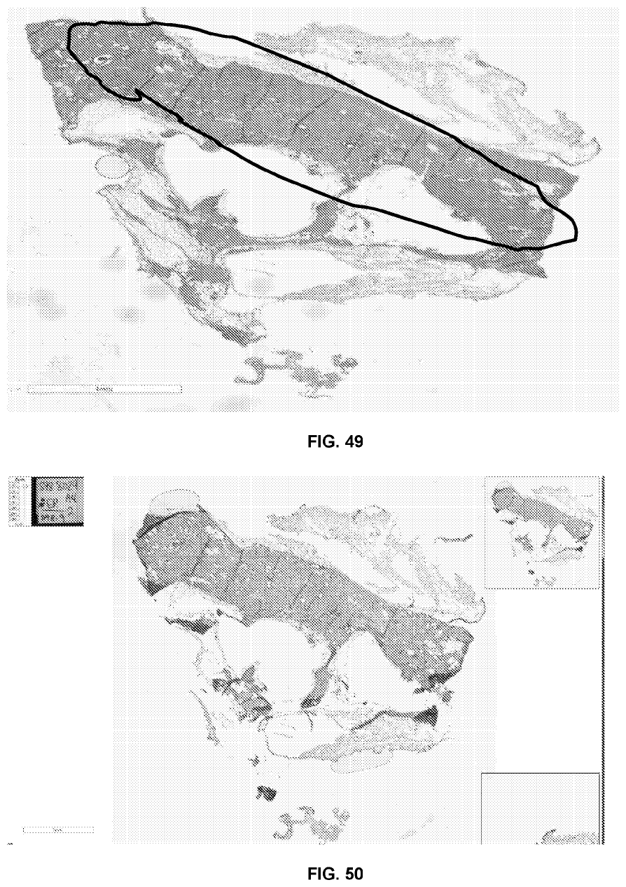

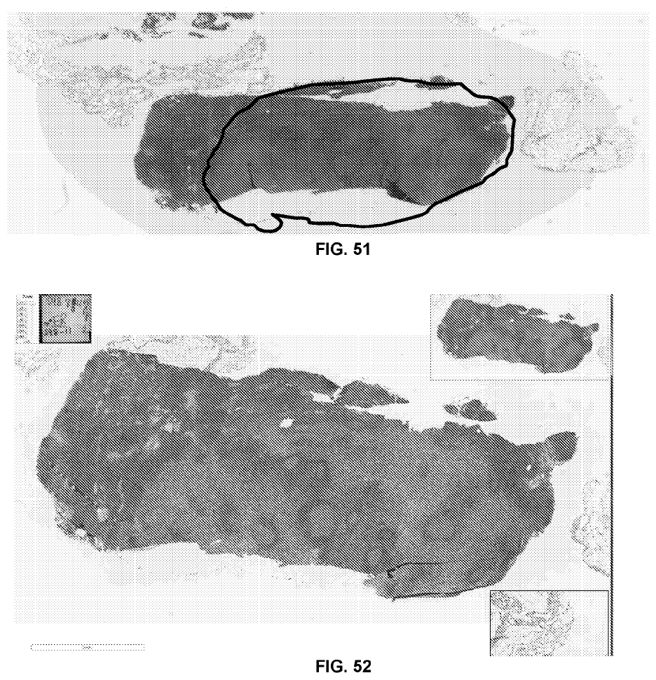

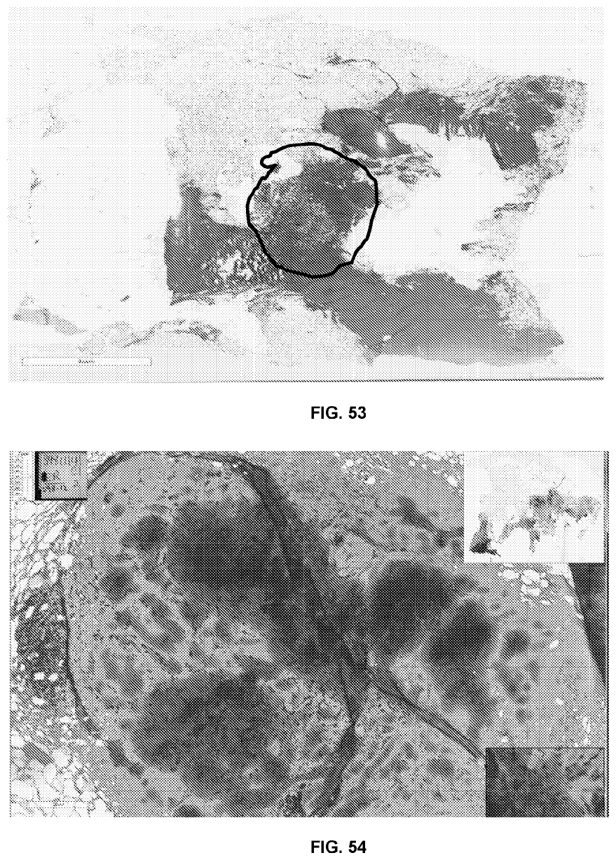

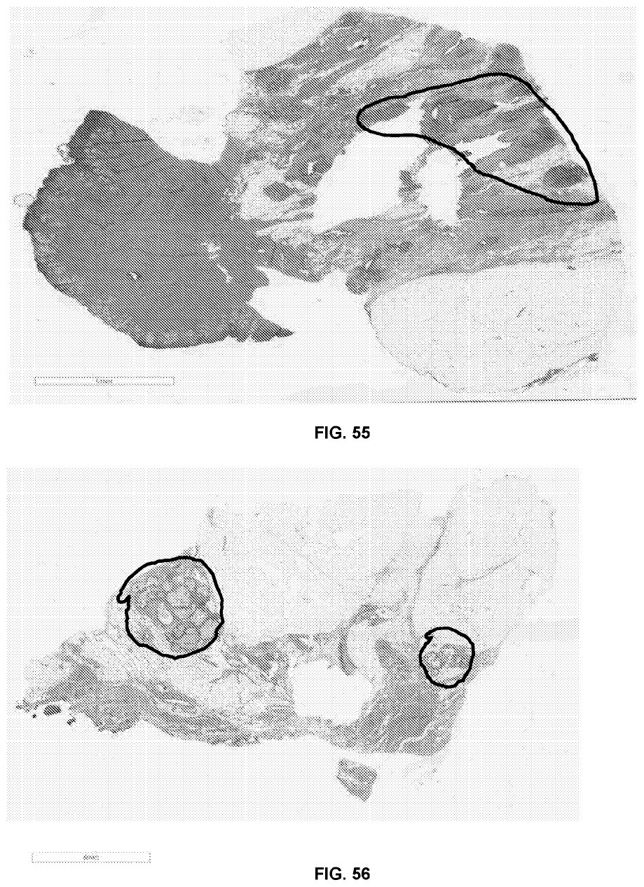

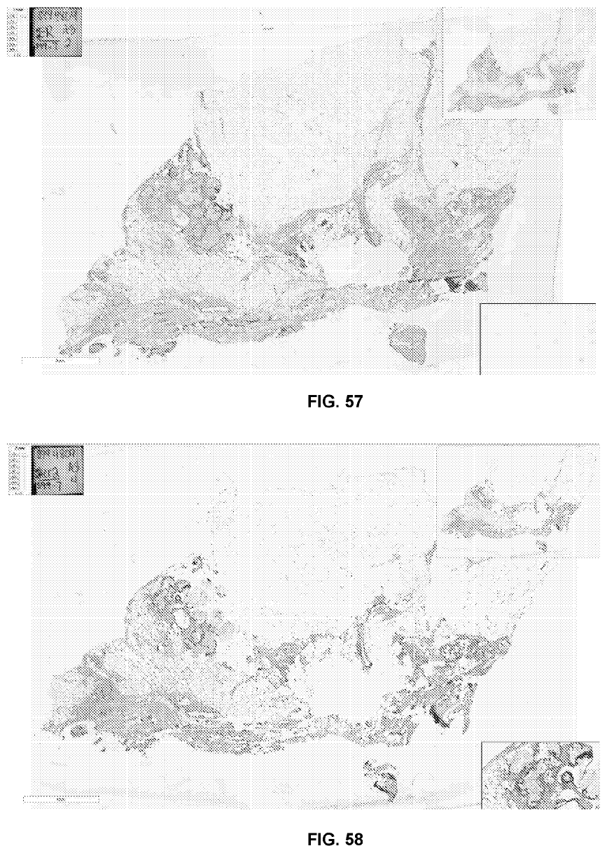

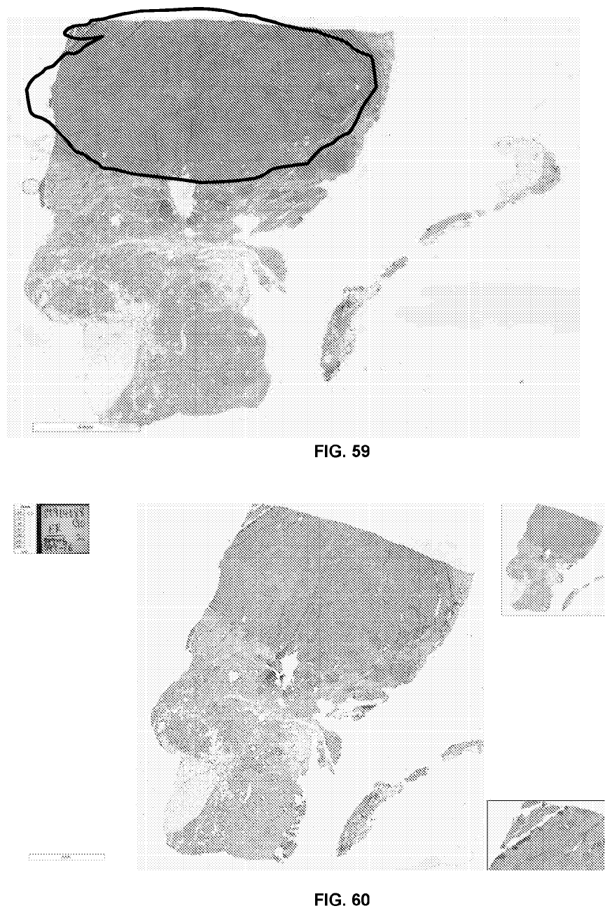

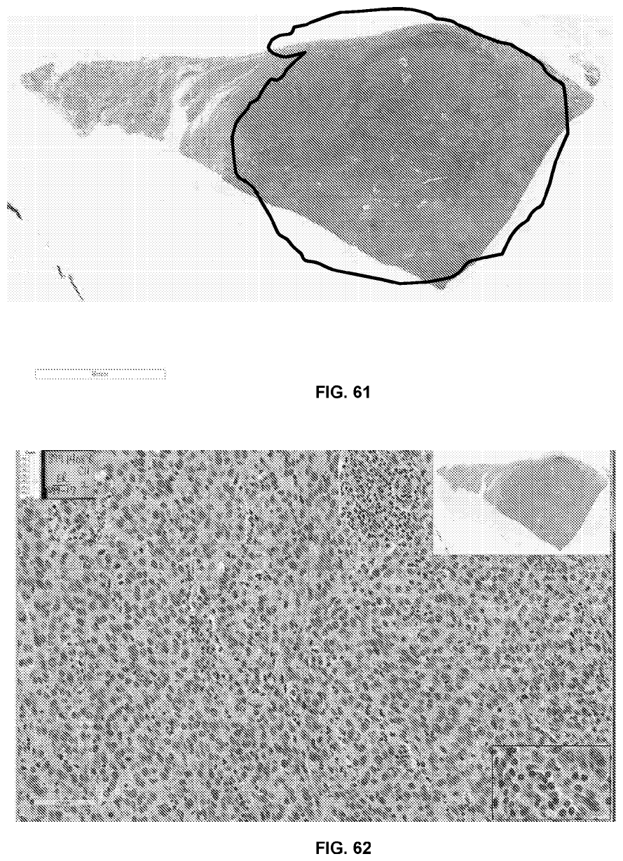

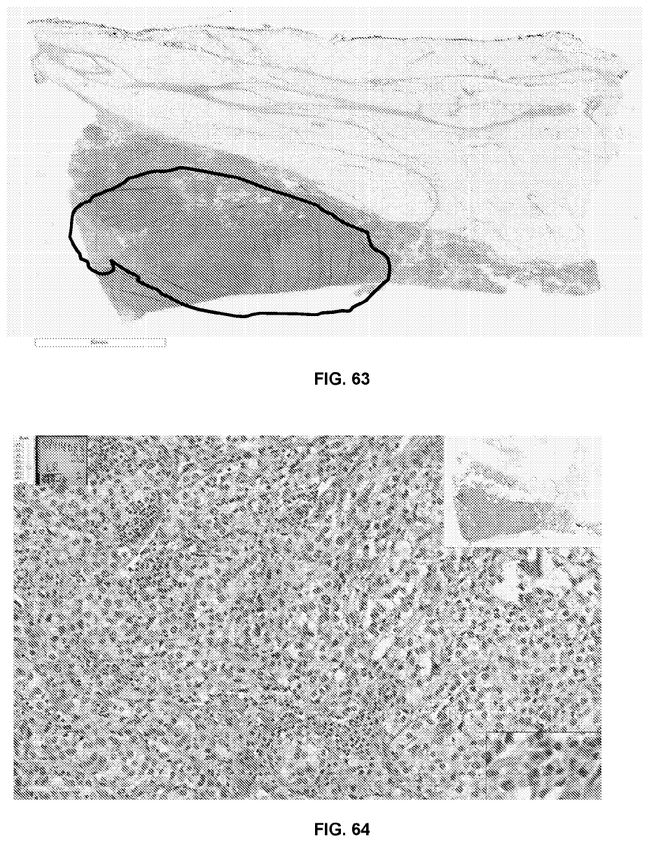

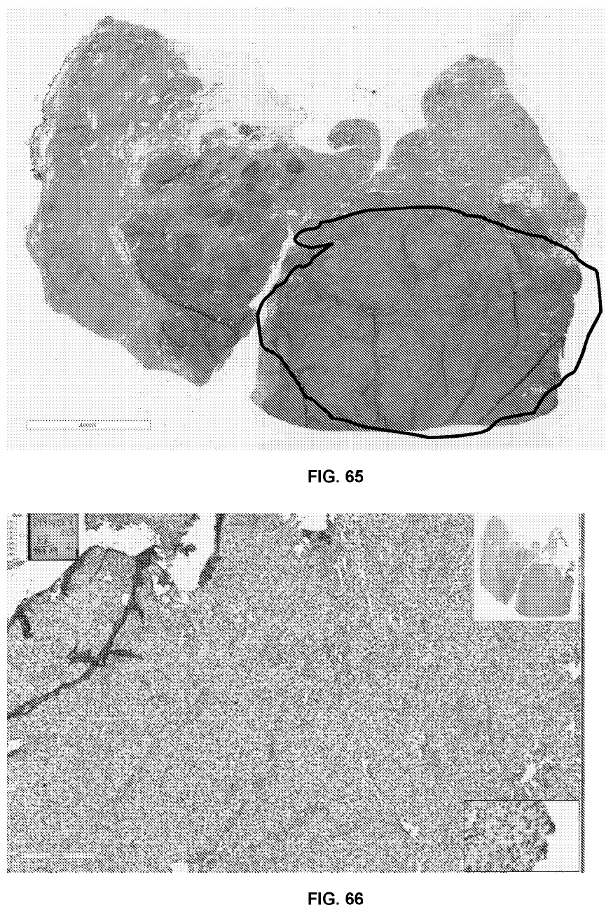

Immunohistochemistry (IHC) is reported in Table 54 for ER, PR, and Her2 with the results listed as either positive (Pos) or negative (Neg) as interpreted by a pathologist based on staining characteristics on specimens T1-T13 (FIG. 40-66—H&E, IHC). Note: IHC for Ki-67 was attempted but was not successful. An “*” in any column of Table 54 indicates the assay was not run. Further results from these same specimens (T1-T13) are reported in the Table 54 columns, i) PAM50 Subtype (RNAseq) and ii) scmod2 subtype (RNAseq), and specify the tumor subtype determined by these two classification methods. The columns ddPCR Subtype, and IntClust Subtype lists the breast tumor subtype for these classification methods on specimens T1-T33.

Comparing agreement of molecular results for specimens T1-T13, IHC: PAM50 showed 11/13 (˜85%), noting that the IHC assessment was imperfect since Ki67 staining failed. IHC: scmod2 showed 13/13 (100%); IHC: ddPCR 11/13 (˜85%); IHC: IntClust, 13/13 (100%). PAM50: scmod2, 11/13 (˜85%); ddPCR: PAM50, 10/13 (˜77%); ddPCR: scmod2, 11/13 (˜85%). Comparing agreement of molecular results for specimens T1-T23, ddPCR: IntClust, showed 20/23 (˜87%) agreement. The column, Notes, provides additional salient information for study specimens. The final five columns: i) Invasive, in-situ, ii) % Tumor, iii) % Normal, iv) Tumor Grade, and v) Comments, correspond to the pathologist reading for each specimen.

### SUMMARY

The disclosed examples evaluated the degree to which robust specimen recovery is possible from the challenging archive. From the S8897 specimens, which were ˜30-year-old FFPE specimens mounted on glass slides and stored at room temperature with no additional specific processing, the tumor gDNA results showed a 95% (18/19) success rate, meaning DNA was extracted, a successful NGS library was built, validated, and sequenced. For LN gDNA the success rate was also 95% (18/19). RNA results were in line with prior work reported by others who have demonstrated excellent recovery and genomic characterization of nucleic acids from archived FFPE, in which extensive fragmentation was present, but effective measurements suggested analytical validity was possible with all cases. Due to the small insert size from the S8897 RNA specimens, a custom ddPCR RT-qPCR assay was developed for tumor subtype calling. The UAMS specimens, which were cut from 20-year-old FFPE blocks stored at room temperature with no additional specific processing, showed a 100% recovery (13/13) for both gDNA and RNA, along with successful NGS.

The disclosed examples further evaluated molecular profiling approaches on the extracted DNA and RNA. Successful low-pass WGS (˜15×) or in some cases ultra-low-pass (˜0.3×) for CNV was achieved for 31/31 specimens and, LN tissue was used as a normal comparator, when available and matched to a specimen. Initially, a DNA panel on eight tumor/LN paired specimens (44 TCGA genes) although successful, revealed a significant degree of subclonal findings, and high false positive rate (FPR) perhaps due to specimen age and fixation. A repeated DNA NGS panel utilizing UMIs was performed on 31 tumor specimens with LN matched pairs (when available) at high coverage (>2000×), for 93 TCGA breast cancer driver genes. The NGS tumor panel library success rate was 30/31 (˜97%). This effectively resolved the high FPR, and brought clarity to the results, many of which reported findings that included COSMIC identifiers, supporting clinical relevance. Since eight S8897 RNA preps were consumed on initial unsuccessful methods, surrogate specimens from UAMS demonstrating extensive fragmentation were utilized. These specimens revealed successful RNA expression profiling for tumor molecular subtyping and, the study team were able to successfully analyze intrinsic subtypes by comparing to IHC for 11/13 tumors using PAM50, 13/13 by scmod2, 11/13 by ddPCR, and 13/13 by IntClust. Additionally, calculation of OncoType gene groups categorized as the Hormonal group (ESR1, PGR, BCL2, SCUBE2), HER2 group (GRB7, HER2), and Proliferation group (MKI67, AURKA, BIRC5, CCNB1, MYBL2) were tallied for the 13 specimens via RNA-seq count data, and compared and contrasted with: PAM50, scmod2, ddPCR, and IHC although unable to analyze the tissues specifically for Ki67. Findings for the Hormonal, HER2, and Proliferation groups via RNA-seq count data were highly concordant with IHC staining results for ER, PR, and HER2, respectively.

The RNA in the disclosed examples was more degraded with an insert size less than 100 nt, thus the need for the custom ddPCR assay for breast tumor subtype determination. The utilization of IntClust subtypes which are based on DNA along with ddPCR assay subtypes based on the highly degraded RNA provided a robust concordance.

The disclosure further provides potential role of molecular characterization of archival FFPE tissue specimens to generate high levels of evidence that might drive patient care. For instance, the favorable prognosis overall of patients in the cohort within S8897 is promising, but raises the question as to why some patients developed distant metastases in spite of an apparently similar clinical-pathological status. The disclosure further demonstrated that the quality of nucleic acid extraction and characterization was satisfactory.

A meticulous nucleic acid extraction with QA/QC metrics, and NGS with DNA for copy number variability (CNV), mutational analysis, and molecular profiling of highly degraded RNA for breast tumor subtyping by RNA-seq for some specimens, and ddPCR RT-qPCR for all specimens is provided herein. The correlation of the ddPCR and IntClust assays for tumor subtyping was complementary and provided robustness given the orthogonal approach of using RNA and DNA for the breast tumor subtype predictive process.

Therefore, examples provided herein demonstrated that high quality molecular profiling can be successfully achieved using FFPE specimens that have been stored for 20-30 years under less-than-ideal conditions, including having been sectioned and stored on glass slides. The analysis has demonstrated technically, there is no barrier for moving forward with larger scale analyses of the remaining specimens in the S8897 “favorable” cohort, and other tissue banks within the many clinical trials organizations that have maintained them.

In summary, a robust specimen recovery for gDNA was achieved for over 90% of specimens. Successful low-depth whole genome DNA sequencing was performed for copy number profiling, and a targeted DNA sequencing approach using Unique Molecular Identifiers (UMIs) was carried out for mutational analysis. RNA recovery showed extensive fragmentation. Some specimens were adequate for RNA-seq analysis. All specimens were adequate for ddPCR-based RT-qPCR analysis and these results were compared with molecular subtyping methods using ER, HER2 and proliferation-related markers. Expression of each of these three sets of gene groups via molecular profiling was highly correlated with matched IHC results in the same specimens.

### EQUIVALENTS

While several inventive aspects have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive aspects described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive aspects described herein. It is, therefore, to be understood that the foregoing aspects are presented by way of example only and that, within the scope of the appended claims and equivalents thereto, inventive aspects may be practiced otherwise than as specifically described and claimed. Inventive aspects of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

What is claimed is:

1. A method of molecular subtyping a cancer sample obtained from a subject, the method comprising:
a) enhanced solubilization of the old and degraded FFPE sample material through the non-discretionary use of mineral oil;
b) digesting the sample with a proteinase;
c) incubating the digested sample from step a) with a DNase;
d) incubating the mixture from step b) with a guanidine salt based buffer;
e) concentrating and isolating the RNA;
f) pre-amplifying; and
g) performing digital droplet PCR.

2. The method of claim 1, wherein the sample is a formalin-fixed and paraffin-embedded sample.

3. The method of claim 1, wherein the sample is at least 5 years old or older.

4. The method of claim 1, wherein the sample is digested with proteinase K.

5. The method of claim 4, wherein the sample is digested at about 65° C. to about 70° C. for about 75 minutes.

6. The method of claim 1, wherein step a) further comprises separating the sample into an aqueous phase by centrifugation and separating the aqueous phase from a residual lysate, where the aqueous phase is used for step b).

7. The method of claim 1, wherein the RNA is concentrated and isolated using a spin column.

8. The method of claim 6, wherein the residual lysate is used for DNA extraction.

9. The method of claim 8, wherein proteinase is incubated with the residual lysate at about 65° C. to about 70° C. for about 13 hours to about 18 hours thereby producing a digested tissue lysate.

10. The method of claim 9, wherein the digested tissue lysate is incubated with an RNase.

11. The method of claim 10, further comprising concentrating and isolating DNA using a spin column.

12. The method of claim 11, further comprising the step of pre-amplifying the isolated DNA and performing ddPCR.

13. The method according to any one of the preceding claims, wherein a target and optionally a reference nucleic acid are quantitated.

14. The method of claim 13, wherein the target nucleic acid or fragment thereof encodes a Estrogen receptor 1 (ESR1), Progesterone receptor (PGR), B-cell lymphoma 2 (BCL2), Signal Peptide, CUB Domain And EGF Like Domain Containing 2 (SCUBE2), human epidermal growth factor receptor 2 (HER2), Growth factor receptor-bound protein 7 (GRB7), Marker Of Proliferation Ki-67 (MKI67), Aurora kinase A (AURKA), Baculoviral IAP Repeat Containing 5 (BIRC5), Cyclin B1 (CCNB1), MYB Proto-Oncogene Like 2 (MYBL2), Thymidine kinase 1 (TK1), or any combination thereof.

15. The method of claim 1, wherein the cancer sample is breast cancer sample.

16. The method of claim 1, wherein the subtype comprises a Luminal A subtype (Lum A), Luminal B subtype (Lum B), HER2 subtype (HER2) or Triple Negative subtype (TN).

17. The method of claim 1 or claim 16, wherein the sample is determined to be Lum A if the level of one or more of target nucleic acid or fragment thereof encoding ESR1, PGR, BCL2, SCUBE2, or any combination thereof, is elevated in the sample.

18. The method of claim 1 or claim 16, the sample is determined to be Lum B if the level of nucleic acid or fragment thereof encoding ESR1, PGR, BCL2, SCUBE2, or any combination thereof, is elevated, and if the level of nucleic acid or fragment thereof encoding MK167, AURKA, BIRC5, CCNB1, MYBL2, TK1, or any combination thereof is elevated in the sample.

19. The method of claim 1 or claim 16, the sample is determined to be HER2 if the level of nucleic acid or fragment thereof encoding HER2, GRB7, or any combination thereof is elevated in the sample.

20. The method of claim 1 or claim 16, the sample is determined to be TN if the level of nucleic acid or fragment thereof encoding MK167, AURKA, BIRC5, CCNB1, MYBL2, TK1, or any combination thereof is elevated in the sample.

21. The method of claim 13 or 14, wherein the amount of target nucleic acid is compared to the subject outcome or a therapeutic response.
